SEQUENCE LISTING (112 sequences)

SEQ ID NO: 1 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - protein, 135 aa, Homo sapiens
MLQMAGQCSQNEYFDSLLHACIPCQLRCSSNTPPLTCQRYCNARSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 3 - protein, 9 aa, synthetic construct
GDVEXNPGP

SEQ ID NO: 4 - protein, 22 aa, synthetic construct
GSGATNFSLLKQAGDVEENPGP

SEQ ID NO: 5 - protein, 23 aa, synthetic construct
GSGQCTNYALLKLAGDVESNPGP

SEQ ID NO: 6 - protein, 25 aa, synthetic construct
GSGVKQTLNFDLLKLAGDVESNPGP

SEQ ID NO: 7 - protein, 7 aa, synthetic construct
SGYSNYK

SEQ ID NO: 8 - protein, 8 aa, synthetic construct
VGTGGIVG

SEQ ID NO: 9 - protein, 13 aa, synthetic construct
GADHGSGNNFVYV

SEQ ID NO: 10 - protein, 8 aa, synthetic construct
GGSISSYY

SEQ ID NO: 11 - protein, 7 aa, synthetic construct
IYKSGNN

SEQ ID NO: 12 - protein, 12 aa, synthetic construct
ADGGNYFDWFDP

SEQ ID NO: 13 - protein, 12 aa, synthetic construct
TLSSGYSNYKVD

SEQ ID NO: 14 - protein, 12 aa, synthetic construct
VGTGGIVGSKGD

SEQ ID NO: 15 - protein, 5 aa, synthetic construct
SYYWN

SEQ ID NO: 16 - protein, 16 aa, synthetic construct
RIYKSGNNIYNPSLQS

SEQ ID NO: 17 - protein, 10 aa, synthetic construct
GGNYFDWFDP

SEQ ID NO: 18 - protein, 116 aa, synthetic construct
QPVLTQPPSASASLGASVTLTCTLSSGYSNYKVDWYQQRPGKGPRFVMRVGTGGIVGSKGDGIPDRFSVLGSGLIRYLTIKNIQEEDESDYHCGADHGSGNNFVYVFGTGTKVTVL

SEQ ID NO: 19 - protein, 118 aa, synthetic construct
QVQLQESGPGLVKPSETLSLTCIVSGGSISSYYWNWIRQPAGKGLEWIGRIYKSGNNIYNPSLQSRVTMSLDTSKNQFSLKLSSVTAADTAVYYCADGGNYFDWFDPWGQGTLVIVSS

SEQ ID NO: 20 - protein, 12 aa, synthetic construct
QSVLYSSYNKNY

SEQ ID NO: 21 - protein, 9 aa, synthetic construct
QQYYSTMYS

SEQ ID NO: 22 - protein, 8 aa, synthetic construct
GFTSSNYW

SEQ ID NO: 23 - protein, 8 aa, synthetic construct
IKHDGSEK

SEQ ID NO: 24 - protein, 10 aa, synthetic construct
ARMRPWYYDL

SEQ ID NO: 25 - protein, 17 aa, synthetic construct
KSSQSVLYSSYNKNYLA

SEQ ID NO: 26 - protein, 7 aa, synthetic construct
WASTRES

SEQ ID NO: 27 - protein, 5 aa, synthetic construct
NYWMS

SEQ ID NO: 28 - protein, 17 aa, synthetic construct
NIKHDGSEKYFVDSVKG

SEQ ID NO: 29 - protein, 8 aa, synthetic construct
MRPWYYDL

SEQ ID NO: 30 - protein, 113 aa, synthetic construct
DIVMTQSPDSLAVSLGERATINCKSSQSVLYSSYNKNYLAWYQQKPGQPPKILIYWASTRESGVPDRFTGSGSGTDFTLTISSLQAEDVAVYYCQQYYSTMYSFGQGTKLEIK

SEQ ID NO: 31 - protein, 117 aa, synthetic construct
EVQLVESGGGLVQPGGSLRLSCAASGFTSSNYWMSWVRQAPGKGLEWVANIKHDGSEKYFVDSVKGRFTISRDNANNSLYLQMNSLRDEDTAVYYCARMRPWYYDLWGRGTLVTVSS

SEQ ID NO: 32 - protein, 7 aa, synthetic construct
SGYSNYR

SEQ ID NO: 33 - protein, 13 aa, synthetic construct
GADHGSGSNFVWV

SEQ ID NO: 34 - protein, 8 aa, synthetic construct
GYTFISYL

SEQ ID NO: 35 - protein, 8 aa, synthetic construct
INPSVGST

SEQ ID NO: 36 - protein, 14 aa, synthetic construct
ARSSRNYWDGAFDI

SEQ ID NO: 37 - protein, 12 aa, synthetic construct
TLSSGYSNYRVD

SEQ ID NO: 38 - protein, 5 aa, synthetic construct
SYLLH

SEQ ID NO: 39 - protein, 17 aa, synthetic construct
IINPSVGSTSYAQKFQG

SEQ ID NO: 40 - protein, 12 aa, synthetic construct
SSRNYWDGAFDI

SEQ ID NO: 41 - protein, 116 aa, synthetic construct
QPVLTQPPSASASLGASVTLTCTLSSGYSNYRVDWYQQRPGKGPRFVMRVGTGGIVGSKGDGIPDRFSVLGSGLNRYLTIKNIQEEDESDYHCGADHGSGSNFVWVFGGGTKLTVL

SEQ ID NO: 42 - protein, 121 aa, synthetic construct
QAQLVQSGAEVKRPGASVKVSCKASGYTFISYLLHWVRQAPGQGLEWMVIINPSVGSTSYAQKFQGRVTMTRDTSTSTLYMELSSLRSEDTAVYYCARSSRNYWDGAFDIWGQGTMVTVSS

SEQ ID NO: 43 - protein, 11 aa, synthetic construct
QSLLHSNGYNY

SEQ ID NO: 44 - protein, 9 aa, synthetic construct
LQALQTPLT

SEQ ID NO: 45 - protein, 7 aa, synthetic construct
MYTSGST

SEQ ID NO: 46 - protein, 16 aa, synthetic construct
ARERGYYYGGGGWIDP

SEQ ID NO: 47 - protein, 16 aa, synthetic construct
RSSQSLLHSNGYNYLD

SEQ ID NO: 48 - protein, 7 aa, synthetic construct
LGSNRAS

SEQ ID NO: 49 - protein, 5 aa, synthetic construct
SYYWC

SEQ ID NO: 50 - protein, 16 aa, synthetic construct
RMYTSGSTNYNPSLKS

SEQ ID NO: 51 - protein, 14 aa, synthetic construct
ERGYYYGGGGWIDP

SEQ ID NO: 52 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCLQALQTPLTFGGGTKVEIK

SEQ ID NO: 53 - protein, 122 aa, synthetic construct
QVQLQESGPGLVKPSETLSLTCTVSGGSISSYYWCWIRQSAGKGLEWIGRMYTSGSTNYNPSLKSRVTMSVDTSKNQFSLKLTSVTAADTAVYYCARERGYYYGGGGWIDPWGQGTLVTVSS

SEQ ID NO: 54 - protein, 5 aa, synthetic construct
SYYWS

SEQ ID NO: 55 - protein, 122 aa, synthetic construct
QVQLQESGPGLVKPSETLSLTCTVSGGSISSYYWSWIRQSAGKGLEWIGRMYTSGSTNYNPSLKSRVTMSVDTSKNQFSLKLTSVTAADTAVYYCARERGYYYGGGGWIDPWGQGTLVTVSS

SEQ ID NO: 56 - protein, 13 aa, synthetic construct
GADHGSGSNFVYV

SEQ ID NO: 57 - protein, 8 aa, synthetic construct
GSTFTSFF

SEQ ID NO: 58 - protein, 8 aa, synthetic construct
INPSGGGT

SEQ ID NO: 59 - protein, 10 aa, synthetic construct
SRGNYEAFDL

SEQ ID NO: 60 - protein, 5 aa, synthetic construct
SFFIH

SEQ ID NO: 61 - protein, 17 aa, synthetic construct
LINPSGGGTGYALKFQG

SEQ ID NO: 62 - protein, 8 aa, synthetic construct
GNYEAFDL

SEQ ID NO: 63 - protein, 116 aa, synthetic construct
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVYVFGTGTKVTVL

SEQ ID NO: 64 - protein, 117 aa, synthetic construct
QVQLVQSGTEVKKPGASVKVSCKASGSTFTSFFIHWVRQAPGQGLEWMGLINPSGGGTGYALKFQGRVTMTRDTSTSTVYMELSSLRSEDTAVYFCSRGNYEAFDLWGHGTMVTVSS

SEQ ID NO: 65 - protein, 10 aa, synthetic construct
GGGGSGGGGS

SEQ ID NO: 66 - protein, 15 aa, synthetic construct
GGGGSGGGGSGGGGS

SEQ ID NO: 67 - protein, 20 aa, synthetic construct
GGGGSGGGGSGGGGSGGGGS

SEQ ID NO: 68 - protein, 249 aa, synthetic construct
QPVLTQPPSASASLGASVTLTCTLSSGYSNYKVDWYQQRPGKGPRFVMRVGTGGIVGSKGDGIPDRFSVLGSGLIRYLTIKNIQEEDESDYHCGADHGSGNNFVYVFGTGTKVTVLGGGGSGGGGSGGGGSQVQLQESGPGLVKPSETLSLTCIVSGGSISSYYWNWIRQPAGKGLEWIGRIYKSGNNIYNPSLQSRVTMSLDTSKNQFSLKLSSVTAADTAVYYCADGGNYFDWFDPWGQGTLVIVSS

SEQ ID NO: 69 - protein, 245 aa, synthetic construct
DIVMTQSPDSLAVSLGERATINCKSSQSVLYSSYNKNYLAWYQQKPGQPPKILIYWASTRESGVPDRFTGSGSGTDFTLTISSLQAEDVAVYYCQQYYSTMYSFGQGTKLEIKGGGGSGGGGSGGGGSEVQLVESGGGLVQPGGSLRLSCAASGFTSSNYWMSWVRQAPGKGLEWVANIKHDGSEKYFVDSVKGRFTISRDNANNSLYLQMNSLRDEDTAVYYCARMRPWYYDLWGRGTLVTVSS

SEQ ID NO: 70 - protein, 252 aa, synthetic construct
QPVLTQPPSASASLGASVTLTCTLSSGYSNYRVDWYQQRPGKGPRFVMRVGTGGIVGSKGDGIPDRFSVLGSGLNRYLTIKNIQEEDESDYHCGADHGSGSNFVWVFGGGTKLTVLGGGGSGGGGSGGGGSQAQLVQSGAEVKRPGASVKVSCKASGYTFISYLLHWVRQAPGQGLEWMVIINPSVGSTSYAQKFQGRVTMTRDTSTSTLYMELSSLRSEDTAVYYCARSSRNYWDGAFDIWGQGTMVTVSS

SEQ ID NO: 71 - protein, 249 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCLQALQTPLTFGGGTKVEIKGGGGSGGGGSGGGGSQVQLQESGPGLVKPSETLSLTCTVSGGSISSYYWSWIRQSAGKGLEWIGRMYTSGSTNYNPSLKSRVTMSVDTSKNQFSLKLTSVTAADTAVYYCARERGYYYGGGGWIDPWGQGTLVTVSS

SEQ ID NO: 72 - protein, 248 aa, synthetic construct
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVYVFGTGTKVTVLGGGGSGGGGSGGGGSQVQLVQSGTEVKKPGASVKVSCKASGSTFTSFFIHWVRQAPGQGLEWMGLINPSGGGTGYALKFQGRVTMTRDTSTSTVYMELSSLRSEDTAVYFCSRGNYEAFDLWGHGTMVTVSS

SEQ ID NO: 73 - protein, 54 aa, Homo sapiens
MLQMAGQCSQNEYFDSLLHACIPCQLRCSSNTPPLTCQRYCNASVTNSVKGTNA

SEQ ID NO: 74 - protein, 21 aa, synthetic construct
MALPVTALLLPLALLLHAARP

SEQ ID NO: 75 - protein, 63 aa, synthetic construct
ATGGCCTTACCAGTGACCGCCTTGCTCCTGCCGCTGGCCTTGCTGCTCCACGCCGCCAGGCCG

SEQ ID NO: 76 - protein, 48 aa, synthetic construct
AKPTTTPAPRPPTPAPTIASQPLSLRPEACRPAAGGAVHTRGLDFACD

SEQ ID NO: 77 - protein, 27 aa, synthetic construct
FWVLVVVGGVLACYSLLVTVAFIIFWV

SEQ ID NO: 78 - protein, 41 aa, synthetic construct
RSKRSRLLHSDYMNMTPRRPGPTRKHYQPYAPPRDFAAYRS

SEQ ID NO: 79 - protein, 22 aa, synthetic construct
ERVQPLEENVGNAARPRFERNK

SEQ ID NO: 80 - protein, 17 aa, synthetic construct
LEENVGNAARPRFERNK

SEQ ID NO: 81 - protein, 8 aa, synthetic construct
RPRFERNK

SEQ ID NO: 82 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 175 aa, synthetic construct
RSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 21 aa, synthetic construct
GSGEGRGSLLTCGDVEENPGP

SEQ ID NO: 85 - protein, 63 aa, synthetic construct
GGCTCAGGTGAGGGGCGCGGGAGCCTGCTGACTTGTGGGGATGTAGAGGAAAATCCTGGTCCT

SEQ ID NO: 86 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 87 - DNA, 489 bp, synthetic construct
  source 1..489: mol_type = other DNA
atgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctcttga

SEQ ID NO: 88 - protein, 499 aa, synthetic construct
QPVLTQPPSASASLGASVTLTCTLSSGYSNYKVDWYQQRPGKGPRFVMRVGTGGIVGSKGDGIPDRFSVLGSGLIRYLTIKNIQEEDESDYHCGADHGSGNNFVYVFGTGTKVTVLGGGGSGGGGSGGGGSQVQLQESGPGLVKPSETLSLTCIVSGGSISSYYWNWIRQPAGKGLEWIGRIYKSGNNIYNPSLQSRVTMSLDTSKNQFSLKLSSVTAADTAVYYCADGGNYFDWFDPWGQGTLVIVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - DNA, 1497 bp, synthetic construct
  source 1..1497: mol_type = other DNA
cagccagtcttgactcaaccaccaagcgcgtccgcaagtttgggtgcttctgttactttgacttgtacactcagctctggttattccaattacaaggtggactggtatcaacagcggcctggaaaggggccgagattcgtcatgcgagtaggcacaggaggtatcgtcggaagtaaaggtgatgggatcccagatcgattctccgtactggggtccggtctgataaggtacctcacaataaagaatatccaggaggaagatgagtccgattaccattgtggggctgaccatggcagcggcaataatttcgtttacgtattcggaaccgggaccaaagtcacggttctcggaggcggtggatctggaggtggaggttccggagggggcggatcacaggtgcagcttcaggagtctggccctggattggtgaaaccttccgaaacgcttagcttgacgtgtatcgtcagtggtggctcaattagcagctattactggaattggatccgacagcccgccgggaaaggtctggaatggatagggagaatatacaaaagcgggaacaacatatataaccccagtttgcagtccagagtaactatgtctctcgatactagtaagaaccagttcagccttaaactcagttccgtgactgccgccgacacggcggtgtactattgcgccgacggagggaattactttgactggtttgacccgtggggtcagggcactcttgtgatagtaagttccgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 90 - protein, 495 aa, synthetic construct
DIVMTQSPDSLAVSLGERATINCKSSQSVLYSSYNKNYLAWYQQKPGQPPKILIYWASTRESGVPDRFTGSGSGTDFTLTISSLQAEDVAVYYCQQYYSTMYSFGQGTKLEIKGGGGSGGGGSGGGGSEVQLVESGGGLVQPGGSLRLSCAASGFTSSNYWMSWVRQAPGKGLEWVANIKHDGSEKYFVDSVKGRFTISRDNANNSLYLQMNSLRDEDTAVYYCARMRPWYYDLWGRGTLVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - DNA, 1485 bp, synthetic construct
  source 1..1485: mol_type = other DNA
gatatagtcatgacccaaagcccagattcccttgcggtatccctgggtgagagagctacgatcaactgcaagagctcacaaagtgtcctctactccagctataataagaattacttggcgtggtatcagcagaaacctggacagccaccaaaaattctcatttactgggcttccactcgggagagcggagtcccggaccgattcacaggttccggttctggaaccgactttaccctgactatttcctccttgcaggcggaagacgttgctgtgtactattgccaacaatattactctacaatgtacagttttgggcagggcaccaaactggaaatcaagggaggcggtggatctggaggtggaggttccggagggggcggatcagaagtccaattggttgaatccggcggagggctggtccaaccgggtggatcacttagacttagctgtgcagcgagcggctttacgtcaagtaactattggatgtcctgggttcgacaagcaccaggcaaaggattggaatgggtagcaaacatcaaacatgacggcagcgaaaaatatttcgttgactccgtcaagggaaggtttacaatatcacgcgacaatgctaacaactctctctaccttcaaatgaattctctccgagatgaagacacagccgtctactattgcgcaagaatgagaccttggtactatgacttgtggggccgaggaacgctcgtgaccgtttcaagtgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 92 - protein, 502 aa, synthetic construct
QPVLTQPPSASASLGASVTLTCTLSSGYSNYRVDWYQQRPGKGPRFVMRVGTGGIVGSKGDGIPDRFSVLGSGLNRYLTIKNIQEEDESDYHCGADHGSGSNFVWVFGGGTKLTVLGGGGSGGGGSGGGGSQAQLVQSGAEVKRPGASVKVSCKASGYTFISYLLHWVRQAPGQGLEWMVIINPSVGSTSYAQKFQGRVTMTRDTSTSTLYMELSSLRSEDTAVYYCARSSRNYWDGAFDIWGQGTMVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - DNA, 1506 bp, synthetic construct
  source 1..1506: mol_type = other DNA
caaccggtactcacacaaccaccgtcagcaagtgcctcccttggcgcttctgttaccctcacatgcaccttgtcttctggctacagcaactaccgagtagattggtaccaacaacgacctggcaaaggacctcggttcgtcatgagggtcgggacaggaggcattgtcgggagcaaaggtgacggaatacctgaccgattctccgtcttggggagtgggcttaaccgctacttgaccatcaagaacatacaagaagaagacgaatctgactatcattgtggtgcggatcatggcagtgggagtaacttcgtttgggtttttggtggaggtacaaaactgactgtacttggaggcggtggatctggaggtggaggttccggagggggcggatcacaggcgcagctcgtgcagagcggtgcggaggtaaagcgcccaggggctagcgtgaaagtttcatgcaaagcctctggctatacgtttattagctacctgctgcactgggttaggcaagcgcccggacaaggattggagtggatggtgataatcaatcccagtgtgggttccacatcatatgcccagaagttccaagggcgagttaccatgactcgagacacctccacatccaccttgtacatggagttgtcatccttgagatccgaagatacggctgtctactattgtgcgcggtccagtcggaactattgggacggagctttcgatatatggggccaagggactatggttactgtgtcttccgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 94 - protein, 499 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCLQALQTPLTFGGGTKVEIKGGGGSGGGGSGGGGSQVQLQESGPGLVKPSETLSLTCTVSGGSISSYYWSWIRQSAGKGLEWIGRMYTSGSTNYNPSLKSRVTMSVDTSKNQFSLKLTSVTAADTAVYYCARERGYYYGGGGWIDPWGQGTLVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - DNA, 1497 bp, synthetic construct
  source 1..1497: mol_type = other DNA
gatattgtcatgacccagtcccctctcagtctcccggtaactccgggcgaaccagcgagtatttcttgtaggagttcccagagcttgctgcatagcaatggctataattacttggactggtacctgcaaaaacccggccagtcaccccaactgctcatctatttgggatcaaaccgggcgagcggcgtacccgacaggttcagtggcagcggttccggaactgacttcactctgaaaattagtcgcgtagaagccgaggatgtgggggtgtactactgcctccaggccctccaaactccccttactttcggcggtggtaccaaagtggagataaaaggaggcggtggatctggaggtggaggttccggagggggcggatcacaggtccagctgcaggaaagtggtcctggccttgtaaagccctccgaaacactctcactgacatgcactgtaagcggagggtcaatttcctcttactactggagctggataagacaaagcgccggcaaaggactcgagtggataggcaggatgtatacctctgggtcaacgaactacaatccgtccctcaaatcacgcgtaacaatgagcgtggacacgtcaaagaaccaattctctttgaagcttactagcgttacggccgcagacacagctgtttactattgcgcgcgcgaacgcggttactattatggtggaggggggtggattgacccatggggtcagggaaccttggtcacagtttcttccgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 96 - protein, 498 aa, synthetic construct
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVYVFGTGTKVTVLGGGGSGGGGSGGGGSQVQLVQSGTEVKKPGASVKVSCKASGSTFTSFFIHWVRQAPGQGLEWMGLINPSGGGTGYALKFQGRVTMTRDTSTSTVYMELSSLRSEDTAVYFCSRGNYEAFDLWGHGTMVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - DNA, 1494 bp, synthetic construct
  source 1..1494: mol_type = other DNA
caacctgtgctgacacagcctccttctgcgtctgcgtcactgggagcttccgtaacactgacctgtacgctctcttctggatatagcaattataaagtagactggtaccaacagagaccggggaaggggccgcgattcgttatgagagttggtaccggtgggatcgtcggatcaaaaggggacgggatcccggatcgattttccgttcttgggtccggtctcaatagatatctgacaattaaaaatatacaagaagaagatgagagcgactaccattgcggagcggaccatggtagtggcagcaactttgtctacgttttcgggaccggcacaaaggtcaccgtactgggaggcggtggatctggaggtggaggttccggagggggcggatcacaggtccagctcgttcagtctggaaccgaggtgaagaagccaggtgcatccgttaaagtgtcttgtaaagcctccggctctacattcacgagtttctttatacactgggtacgccaggcgcctggccagggtcttgaatggatgggccttattaatccatccggtggaggtacaggctatgcacttaagttccaaggcagggtcaccatgacgcgcgacacgtcaacatcaactgtgtatatggaactctcttctcttcgctctgaagacacggctgtttatttctgcagtcgaggaaattacgaggcattcgacctttgggggcacggaactatggtgacagtaagctcagcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 98 - protein, 523 aa, synthetic construct
MALPVTALLLPLALLLHAARPQPVLTQPPSASASLGASVTLTCTLSSGYSNYRVDWYQQRPGKGPRFVMRVGTGGIVGSKGDGIPDRFSVLGSGLNRYLTIKNIQEEDESDYHCGADHGSGSNFVWVFGGGTKLTVLGGGGSGGGGSGGGGSQAQLVQSGAEVKRPGASVKVSCKASGYTFISYLLHWVRQAPGQGLEWMVIINPSVGSTSYAQKFQGRVTMTRDTSTSTLYMELSSLRSEDTAVYYCARSSRNYWDGAFDIWGQGTMVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 520 aa, synthetic construct
MALPVTALLLPLALLLHAARPDIVMTQSPLSLPVTPGEPASISCRSSQSLLHSNGYNYLDWYLQKPGQSPQLLIYLGSNRASGVPDRFSGSGSGTDFTLKISRVEAEDVGVYYCLQALQTPLTFGGGTKVEIKGGGGSGGGGSGGGGSQVQLQESGPGLVKPSETLSLTCTVSGGSISSYYWSWIRQSAGKGLEWIGRMYTSGSTNYNPSLKSRVTMSVDTSKNQFSLKLTSVTAADTAVYYCARERGYYYGGGGWIDPWGQGTLVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 520 aa, synthetic construct
MALPVTALLLPLALLLHAARPQPVLTQPPSASASLGASVTLTCTLSSGYSNYKVDWYQQRPGKGPRFVMRVGTGGIVGSKGDGIPDRFSVLGSGLIRYLTIKNIQEEDESDYHCGADHGSGNNFVYVFGTGTKVTVLGGGGSGGGGSGGGGSQVQLQESGPGLVKPSETLSLTCIVSGGSISSYYWNWIRQPAGKGLEWIGRIYKSGNNIYNPSLQSRVTMSLDTSKNQFSLKLSSVTAADTAVYYCADGGNYFDWFDPWGQGTLVIVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 519 aa, synthetic construct
MALPVTALLLPLALLLHAARPQPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVYVFGTGTKVTVLGGGGSGGGGSGGGGSQVQLVQSGTEVKKPGASVKVSCKASGSTFTSFFIHWVRQAPGQGLEWMGLINPSGGGTGYALKFQGRVTMTRDTSTSTVYMELSSLRSEDTAVYFCSRGNYEAFDLWGHGTMVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 516 aa, synthetic construct
MALPVTALLLPLALLLHAARPDIVMTQSPDSLAVSLGERATINCKSSQSVLYSSYNKNYLAWYQQKPGQPPKILIYWASTRESGVPDRFTGSGSGTDFTLTISSLQAEDVAVYYCQQYYSTMYSFGQGTKLEIKGGGGSGGGGSGGGGSEVQLVESGGGLVQPGGSLRLSCAASGFTSSNYWMSWVRQAPGKGLEWVANIKHDGSEKYFVDSVKGRFTISRDNANNSLYLQMNSLRDEDTAVYYCARMRPWYYDLWGRGTLVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - DNA, 1569 bp, synthetic construct
  source 1..1569: mol_type = other DNA
atggctctgccagtgactgcactgctgctgccactggccctgctgctgcacgcagctcgacctcaaccggtactcacacaaccaccgtcagcaagtgcctcccttggcgcttctgttaccctcacatgcaccttgtcttctggctacagcaactaccgagtagattggtaccaacaacgacctggcaaaggacctcggttcgtcatgagggtcgggacaggaggcattgtcgggagcaaaggtgacggaatacctgaccgattctccgtcttggggagtgggcttaaccgctacttgaccatcaagaacatacaagaagaagacgaatctgactatcattgtggtgcggatcatggcagtgggagtaacttcgtttgggtttttggtggaggtacaaaactgactgtacttggaggcggtggatctggaggtggaggttccggagggggcggatcacaggcgcagctcgtgcagagcggtgcggaggtaaagcgcccaggggctagcgtgaaagtttcatgcaaagcctctggctatacgtttattagctacctgctgcactgggttaggcaagcgcccggacaaggattggagtggatggtgataatcaatcccagtgtgggttccacatcatatgcccagaagttccaagggcgagttaccatgactcgagacacctccacatccaccttgtacatggagttgtcatccttgagatccgaagatacggctgtctactattgtgcgcggtccagtcggaactattgggacggagctttcgatatatggggccaagggactatggttactgtgtcttccgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 104 - DNA, 2121 bp, synthetic construct
  source 1..2121: mol_type = other DNA
atggctctgccagtgactgcactgctgctgccactggccctgctgctgcacgcagctcgacctcaaccggtactcacacaaccaccgtcagcaagtgcctcccttggcgcttctgttaccctcacatgcaccttgtcttctggctacagcaactaccgagtagattggtaccaacaacgacctggcaaaggacctcggttcgtcatgagggtcgggacaggaggcattgtcgggagcaaaggtgacggaatacctgaccgattctccgtcttggggagtgggcttaaccgctacttgaccatcaagaacatacaagaagaagacgaatctgactatcattgtggtgcggatcatggcagtgggagtaacttcgtttgggtttttggtggaggtacaaaactgactgtacttggaggcggtggatctggaggtggaggttccggagggggcggatcacaggcgcagctcgtgcagagcggtgcggaggtaaagcgcccaggggctagcgtgaaagtttcatgcaaagcctctggctatacgtttattagctacctgctgcactgggttaggcaagcgcccggacaaggattggagtggatggtgataatcaatcccagtgtgggttccacatcatatgcccagaagttccaagggcgagttaccatgactcgagacacctccacatccaccttgtacatggagttgtcatccttgagatccgaagatacggctgtctactattgtgcgcggtccagtcggaactattgggacggagctttcgatatatggggccaagggactatggttactgtgtcttccgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggggctcaggtgaggggcgcgggagcctgctgacttgtggggatgtagaggaaaatcctggtcctatgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctcttga

SEQ ID NO: 105 - DNA, 1560 bp, synthetic construct
  source 1..1560: mol_type = other DNA
atggctctgccagtgactgcactgctgctgccactggccctgctgctgcacgcagctcgacctgatattgtcatgacccagtcccctctcagtctcccggtaactccgggcgaaccagcgagtatttcttgtaggagttcccagagcttgctgcatagcaatggctataattacttggactggtacctgcaaaaacccggccagtcaccccaactgctcatctatttgggatcaaaccgggcgagcggcgtacccgacaggttcagtggcagcggttccggaactgacttcactctgaaaattagtcgcgtagaagccgaggatgtgggggtgtactactgcctccaggccctccaaactccccttactttcggcggtggtaccaaagtggagataaaaggaggcggtggatctggaggtggaggttccggagggggcggatcacaggtccagctgcaggaaagtggtcctggccttgtaaagccctccgaaacactctcactgacatgcactgtaagcggagggtcaatttcctcttactactggagctggataagacaaagcgccggcaaaggactcgagtggataggcaggatgtatacctctgggtcaacgaactacaatccgtccctcaaatcacgcgtaacaatgagcgtggacacgtcaaagaaccaattctctttgaagcttactagcgttacggccgcagacacagctgtttactattgcgcgcgcgaacgcggttactattatggtggaggggggtggattgacccatggggtcagggaaccttggtcacagtttcttccgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 106 - DNA, 2112 bp, synthetic construct
  source 1..2112: mol_type = other DNA
atggctctgccagtgactgcactgctgctgccactggccctgctgctgcacgcagctcgacctgatattgtcatgacccagtcccctctcagtctcccggtaactccgggcgaaccagcgagtatttcttgtaggagttcccagagcttgctgcatagcaatggctataattacttggactggtacctgcaaaaacccggccagtcaccccaactgctcatctatttgggatcaaaccgggcgagcggcgtacccgacaggttcagtggcagcggttccggaactgacttcactctgaaaattagtcgcgtagaagccgaggatgtgggggtgtactactgcctccaggccctccaaactccccttactttcggcggtggtaccaaagtggagataaaaggaggcggtggatctggaggtggaggttccggagggggcggatcacaggtccagctgcaggaaagtggtcctggccttgtaaagccctccgaaacactctcactgacatgcactgtaagcggagggtcaatttcctcttactactggagctggataagacaaagcgccggcaaaggactcgagtggataggcaggatgtatacctctgggtcaacgaactacaatccgtccctcaaatcacgcgtaacaatgagcgtggacacgtcaaagaaccaattctctttgaagcttactagcgttacggccgcagacacagctgtttactattgcgcgcgcgaacgcggttactattatggtggaggggggtggattgacccatggggtcagggaaccttggtcacagtttcttccgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggggctcaggtgaggggcgcgggagcctgctgacttgtggggatgtagaggaaaatcctggtcctatgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctcttga

SEQ ID NO: 107 - DNA, 1560 bp, synthetic construct
  source 1..1560: mol_type = other DNA
atggctctgccagtgactgcactgctgctgccactggccctgctgctgcacgcagctcgacctcagccagtcttgactcaaccaccaagcgcgtccgcaagtttgggtgcttctgttactttgacttgtacactcagctctggttattccaattacaaggtggactggtatcaacagcggcctggaaaggggccgagattcgtcatgcgagtaggcacaggaggtatcgtcggaagtaaaggtgatgggatcccagatcgattctccgtactggggtccggtctgataaggtacctcacaataaagaatatccaggaggaagatgagtccgattaccattgtggggctgaccatggcagcggcaataatttcgtttacgtattcggaaccgggaccaaagtcacggttctcggaggcggtggatctggaggtggaggttccggagggggcggatcacaggtgcagcttcaggagtctggccctggattggtgaaaccttccgaaacgcttagcttgacgtgtatcgtcagtggtggctcaattagcagctattactggaattggatccgacagcccgccgggaaaggtctggaatggatagggagaatatacaaaagcgggaacaacatatataaccccagtttgcagtccagagtaactatgtctctcgatactagtaagaaccagttcagccttaaactcagttccgtgactgccgccgacacggcggtgtactattgcgccgacggagggaattactttgactggtttgacccgtggggtcagggcactcttgtgatagtaagttccgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 108 - DNA, 2112 bp, synthetic construct
  source 1..2112: mol_type = other DNA
atggctctgccagtgactgcactgctgctgccactggccctgctgctgcacgcagctcgacctcagccagtcttgactcaaccaccaagcgcgtccgcaagtttgggtgcttctgttactttgacttgtacactcagctctggttattccaattacaaggtggactggtatcaacagcggcctggaaaggggccgagattcgtcatgcgagtaggcacaggaggtatcgtcggaagtaaaggtgatgggatcccagatcgattctccgtactggggtccggtctgataaggtacctcacaataaagaatatccaggaggaagatgagtccgattaccattgtggggctgaccatggcagcggcaataatttcgtttacgtattcggaaccgggaccaaagtcacggttctcggaggcggtggatctggaggtggaggttccggagggggcggatcacaggtgcagcttcaggagtctggccctggattggtgaaaccttccgaaacgcttagcttgacgtgtatcgtcagtggtggctcaattagcagctattactggaattggatccgacagcccgccgggaaaggtctggaatggatagggagaatatacaaaagcgggaacaacatatataaccccagtttgcagtccagagtaactatgtctctcgatactagtaagaaccagttcagccttaaactcagttccgtgactgccgccgacacggcggtgtactattgcgccgacggagggaattactttgactggtttgacccgtggggtcagggcactcttgtgatagtaagttccgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggggctcaggtgaggggcgcgggagcctgctgacttgtggggatgtagaggaaaatcctggtcctatgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctcttga

SEQ ID NO: 109 - DNA, 1557 bp, synthetic construct
  source 1..1557: mol_type = other DNA
atggctctgccagtgactgcactgctgctgccactggccctgctgctgcacgcagctcgacctcaacctgtgctgacacagcctccttctgcgtctgcgtcactgggagcttccgtaacactgacctgtacgctctcttctggatatagcaattataaagtagactggtaccaacagagaccggggaaggggccgcgattcgttatgagagttggtaccggtgggatcgtcggatcaaaaggggacgggatcccggatcgattttccgttcttgggtccggtctcaatagatatctgacaattaaaaatatacaagaagaagatgagagcgactaccattgcggagcggaccatggtagtggcagcaactttgtctacgttttcgggaccggcacaaaggtcaccgtactgggaggcggtggatctggaggtggaggttccggagggggcggatcacaggtccagctcgttcagtctggaaccgaggtgaagaagccaggtgcatccgttaaagtgtcttgtaaagcctccggctctacattcacgagtttctttatacactgggtacgccaggcgcctggccagggtcttgaatggatgggccttattaatccatccggtggaggtacaggctatgcacttaagttccaaggcagggtcaccatgacgcgcgacacgtcaacatcaactgtgtatatggaactctcttctcttcgctctgaagacacggctgtttatttctgcagtcgaggaaattacgaggcattcgacctttgggggcacggaactatggtgacagtaagctcagcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 110 - DNA, 2109 bp, synthetic construct
  source 1..2109: mol_type = other DNA
atggctctgccagtgactgcactgctgctgccactggccctgctgctgcacgcagctcgacctcaacctgtgctgacacagcctccttctgcgtctgcgtcactgggagcttccgtaacactgacctgtacgctctcttctggatatagcaattataaagtagactggtaccaacagagaccggggaaggggccgcgattcgttatgagagttggtaccggtgggatcgtcggatcaaaaggggacgggatcccggatcgattttccgttcttgggtccggtctcaatagatatctgacaattaaaaatatacaagaagaagatgagagcgactaccattgcggagcggaccatggtagtggcagcaactttgtctacgttttcgggaccggcacaaaggtcaccgtactgggaggcggtggatctggaggtggaggttccggagggggcggatcacaggtccagctcgttcagtctggaaccgaggtgaagaagccaggtgcatccgttaaagtgtcttgtaaagcctccggctctacattcacgagtttctttatacactgggtacgccaggcgcctggccagggtcttgaatggatgggccttattaatccatccggtggaggtacaggctatgcacttaagttccaaggcagggtcaccatgacgcgcgacacgtcaacatcaactgtgtatatggaactctcttctcttcgctctgaagacacggctgtttatttctgcagtcgaggaaattacgaggcattcgacctttgggggcacggaactatggtgacagtaagctcagcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggggctcaggtgaggggcgcgggagcctgctgacttgtggggatgtagaggaaaatcctggtcctatgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctcttga

SEQ ID NO: 111 - DNA, 1548 bp, synthetic construct
  source 1..1548: mol_type = other DNA
atggctctgccagtgactgcactgctgctgccactggccctgctgctgcacgcagctcgacctgatatagtcatgacccaaagcccagattcccttgcggtatccctgggtgagagagctacgatcaactgcaagagctcacaaagtgtcctctactccagctataataagaattacttggcgtggtatcagcagaaacctggacagccaccaaaaattctcatttactgggcttccactcgggagagcggagtcccggaccgattcacaggttccggttctggaaccgactttaccctgactatttcctccttgcaggcggaagacgttgctgtgtactattgccaacaatattactctacaatgtacagttttgggcagggcaccaaactggaaatcaagggaggcggtggatctggaggtggaggttccggagggggcggatcagaagtccaattggttgaatccggcggagggctggtccaaccgggtggatcacttagacttagctgtgcagcgagcggctttacgtcaagtaactattggatgtcctgggttcgacaagcaccaggcaaaggattggaatgggtagcaaacatcaaacatgacggcagcgaaaaatatttcgttgactccgtcaagggaaggtttacaatatcacgcgacaatgctaacaactctctctaccttcaaatgaattctctccgagatgaagacacagccgtctactattgcgcaagaatgagaccttggtactatgacttgtggggccgaggaacgctcgtgaccgtttcaagtgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 112 - DNA, 2100 bp, synthetic construct
  source 1..2100: mol_type = other DNA
atggctctgccagtgactgcactgctgctgccactggccctgctgctgcacgcagctcgacctgatatagtcatgacccaaagcccagattcccttgcggtatccctgggtgagagagctacgatcaactgcaagagctcacaaagtgtcctctactccagctataataagaattacttggcgtggtatcagcagaaacctggacagccaccaaaaattctcatttactgggcttccactcgggagagcggagtcccggaccgattcacaggttccggttctggaaccgactttaccctgactatttcctccttgcaggcggaagacgttgctgtgtactattgccaacaatattactctacaatgtacagttttgggcagggcaccaaactggaaatcaagggaggcggtggatctggaggtggaggttccggagggggcggatcagaagtccaattggttgaatccggcggagggctggtccaaccgggtggatcacttagacttagctgtgcagcgagcggctttacgtcaagtaactattggatgtcctgggttcgacaagcaccaggcaaaggattggaatgggtagcaaacatcaaacatgacggcagcgaaaaatatttcgttgactccgtcaagggaaggtttacaatatcacgcgacaatgctaacaactctctctaccttcaaatgaattctctccgagatgaagacacagccgtctactattgcgcaagaatgagaccttggtactatgacttgtggggccgaggaacgctcgtgaccgtttcaagtgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagaccgggtcccactcgcaaacactaccagccctatgctccaccccgggacttcgcggcctacaggtcagagagagtgcagcccctggaagagaatgtcggcaacgctgcccgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggggctcaggtgaggggcgcgggagcctgctgacttgtggggatgtagaggaaaatcctggtcctatgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctcttga

(19) United States
(12) Patent Application Publication
(10) Pub. No.: US 2026/0207747 A1
(43) Pub. Date: Jul. 23, 2026

(54) ANTI-BCMA ANTIBODY OR ANTIGEN-BINDING FRAGMENT THEREOF, AND CHIMERIC ANTIGEN RECEPTOR COMPRISING SAME

(71) Applicant: Artiva Biotherapeutics, Inc., San Diego, CA (US)

(72) Inventors: Srinivas Sai Somanchi, San Diego, CA (US); Adam Louis Corper, San Diego, CA (US); Kun Yu Teng, San Diego, CA (US); Peter Flynn, San Diego, CA (US)

(21) Appl. No.: 19/134,363

(22) PCT Filed: Dec. 1, 2023
(86) PCT No.: PCT/US2023/082160

§ 371 Date: May 30, 2025

Related U.S. Application Data
(60) Provisional application No. 63/429,910, filed on Dec. 2, 2022.

(51) Int. Cl. A61K 40/42; A61K 39/395; A61K 40/11; A61K 40/15; A61K 40/31; A61K 40/33; A61P 35/00; C07K 14/705; C07K 16/28; C12N 5/0783; C12N 15/86

(52) U.S. Cl. CPC A61K 40/4215; A61K 39/3955; A61K 40/11; A61K 40/15; A61K 40/31; A61K 40/33; A61P 35/00; C07K 14/7051; C07K 14/70517; C07K 14/70521; C07K 14/70575; C07K 16/2878; C12N 5/0646; C12N 15/86; C07K 2317/21; C07K 2317/24; C07K 2317/622; C07K 2319/02; C12N 2510/00; C12N 2740/15043

(57) ABSTRACT

Provided herein are, among other things, are anti-BCMA antibodies and antigenbinding fragments thereof, chimeric antigen receptors comprising the same, natural killer cells expressing the chimeric antigen receptors. Also provided herein are pharmaceutical compositions comprising the anti-BCMA antibodies and antigen-binding fragments thereof, chimeric antigen receptors comprising the same, or natural killer cells. Also provided herein are methods of treatment, e.g., of BCMA+ cancers, comprising administering the anti-BCMA antibodies and antigen-binding fragments thereof, chimeric antigen receptors comprising the same, or natural killer cells described herein.

### CLAIM OF PRIORITY

This application claims the benefit of U.S. Provisional Application Ser. No. 63/429,910, filed on Dec. 2, 2022. The entire contents of the foregoing are incorporated herein by reference.

### SEQUENCE LISTING

This application contains a Sequence Listing that has been submitted electronically as an XML file named 49755-0037W01_SL_ST26.xml. The XML file, created on Nov. 30, 2023, is 132,857 bytes in size. The material in the XML file is hereby incorporated by reference in its entirety.

### TECHNICAL FIELD

Provided herein are, among other things, are anti-BCMA antibodies and antigen-binding fragments thereof, chimeric antigen receptors comprising the same, natural killer cells expressing the chimeric antigen receptors. Also provided herein are pharmaceutical compositions comprising the anti-BCMA antibodies and antigen-binding fragments thereof, chimeric antigen receptors comprising the same, or natural killer cells. Also provided herein are methods of treatment, e.g., of BCMA+ cancers, comprising administering the anti-BCMA antibodies and antigen-binding fragments thereof, chimeric antigen receptors comprising the same, or natural killer cells described herein.

### BACKGROUND

BCMA is an attractive tumor associated antigen to target cancer cells such as multiple myeloma cells. While CAR-T approaches exist, the autologous CAR-T approach is not widely accessible to patients in need and the risks of cytokine release syndrome (CRS) and neurotoxicity of CAR-T are substantial.

### SUMMARY

There is a significant need to develop BCMA targeted approaches using allogeneic and safer cell therapeutics, such as CAR-NK cells. Thus, provided herein are, among other things, BCMA-binding agents (e.g., anti-BCMA antibodies and antigen-binding fragments thereof), chimeric antigen receptors comprising the same, and natural killer cells expressing the chimeric antigen receptors. Also provided herein are pharmaceutical compositions comprising the BCMA-binding agents (e.g., anti-BCMA antibodies and antigen-binding fragments thereof), chimeric antigen receptors comprising the same, or natural killer cells. Also provided herein are methods of treatment. e.g., of BCMA+ cancers, comprising administering the BCMA-binding agents (e.g., anti-BCMA antibodies and antigen-binding fragments thereof), chimeric antigen receptors comprising the same, or natural killer cells described herein.

The BCMA-binding agents (e.g., anti-BCMA antibodies and antigen-binding fragments thereof) described herein, can be useful, among other things, for maximizing BCMA-directed tumor lysis. For example, by minimizing inhibition by soluble BCMA, e.g., by having a high avidity for membrane bound BCMA, but a lower affinity for monomeric BCMA.

Thus, provided herein, among other things, is an anti-BCMA antibody or antigen binding fragment thereof comprising: a CDR-L1 having the amino acid sequence of SEQ ID NO: 7, a CDR-L2 having the amino acid sequence of SEQ ID NO: 8, a CDR-L3 having the amino acid sequence of SEQ ID NO: 9, a CDR-H1 having the amino acid sequence of SEQ ID NO: 10, a CDR-H2 having the amino acid sequence of SEQ ID NO: 11, and a CDR-H3 having the amino acid sequence of SEQ ID NO: 12. In some embodiments, the CDR-L1, CDR-L2, CDR-L3, CDR-H1, CDR-H2, and CDR-H3 are determined by IMGT numbering.

Also provided herein are anti-BCMA antibodies or antigen binding fragments thereof comprising: a CDR-L1 having the amino acid sequence of SEQ ID NO: 13, a CDR-L2 having the amino acid sequence of SEQ ID NO: 14, a CDR-L3 having the amino acid sequence of SEQ ID NO: 9, a CDR-H1 having the amino acid sequence of SEQ ID NO: 15, a CDR-H2 having the amino acid sequence of SEQ ID NO: 16, and a CDR-H3 having the amino acid sequence of SEQ ID NO: 17. In some embodiments, the CDR-L1, CDR-L2. CDR-L3, CDR-H1, CDR-H2, and CDR-H3 are determined by Kabat numbering.

In some embodiments, the antibody or antigen binding fragment thereof comprises a light chain variable (V_{L}) region comprising the amino acid sequence of SEQ ID NO: 18 and a heavy chain variable (V_{H}) region comprising the amino acid sequence of SEQ ID NO: 19. In some embodiments, the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 18 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 19. In some embodiments, the antibody or antigen binding fragment thereof comprises a V_{L} region comprising the amino acid sequence of SEQ ID NO: 18, optionally with up to 10 (e.g., 1, 2, 3, 4, 5, 6, 7, 8, 9 or 10) substitutions (e.g., conservative substitutions) and a V_{H} region comprising the amino acid sequence of SEQ ID NO: 19, optionally with up to 10 (e.g., 1, 2, 3, 4, 5, 6, 7, 8, 9, or 10) substitutions (e.g., conservative substitutions).

Also provided herein are anti-BCMA antibodies or antigen binding fragments thereof comprising: a CDR-L1 having the amino acid sequence of SEQ ID NO: 20, a CDR-L2 having the amino acid sequence WAS, a CDR-L3 having the amino acid sequence of SEQ ID NO: 21, a CDR-H1 having the amino acid sequence of SEQ ID NO: 22, a CDR-H2 having the amino acid sequence of SEQ ID NO: 23, and a CDR-H3 having the amino acid sequence of SEQ ID NO: 24. In some embodiments, the CDR-L1, CDR-L2, CDR-L3, CDR-H1, CDR-H2, and CDR-H3 are determined by IMGT numbering.

Also provided herein are anti-BCMA antibodies or antigen binding fragments thereof comprising: a CDR-L1 having the amino acid sequence of SEQ ID NO: 25, a CDR-L2 having the amino acid sequence of SEQ ID NO: 26, a CDR-L3 having the amino acid sequence of SEQ ID NO: 21, a CDR-H1 having the amino acid sequence of SEQ ID NO: 27, a CDR-H2 having the amino acid sequence of SEQ ID NO: 28, and a CDR-H3 having the amino acid sequence of SEQ ID NO: 29. In some embodiments, the CDR-L1, CDR-L2, CDR-L3. CDR-H1, CDR-H2, and CDR-H3 are determined by Kabat numbering.

In some embodiments, the antibody or antigen binding fragment thereof comprises a light chain variable (V_{L}) region comprising the amino acid sequence of SEQ ID NO: 30 and a heavy chain variable (V_{H}) region comprising the amino acid sequence of SEQ ID NO: 31. In some embodiments, the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 30 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 31. In some embodiments, the antibody or antigen binding fragment thereof comprises a V_{L} region comprising the amino acid sequence of SEQ ID NO: 30, optionally with up to 10 (e.g., 1, 2, 3, 4, 5, 6, 7, 8, 9 or 10) substitutions (e.g., conservative substitutions) and a V_{H} region comprising the amino acid sequence of SEQ ID NO: 31, optionally with up to 10 (e.g., 1, 2, 3, 4, 5, 6, 7, 8, 9, or 10) substitutions (e.g., conservative substitutions).

Also provided herein are anti-BCMA antibodies or antigen binding fragments thereof comprising: a CDR-L1 having the amino acid sequence of SEQ ID NO: 32, a CDR-L2 having the amino acid sequence SEQ ID NO: 8, a CDR-L3 having the amino acid sequence of SEQ ID NO: 33, a CDR-H1 having the amino acid sequence of SEQ ID NO: 34, a CDR-H2 having the amino acid sequence of SEQ ID NO: 35, and a CDR-H3 having the amino acid sequence of SEQ ID NO: 36. In some embodiments, the CDR-L1, CDR-L2, CDR-L3. CDR-H1, CDR-H2, and CDR-H3 are determined by IMGT numbering.

Also provided herein are anti-BCMA antibodies or antigen binding fragments thereof comprising: a CDR-L1 having the amino acid sequence of SEQ ID NO: 37, a CDR-L2 having the amino acid sequence of SEQ ID NO: 14, a CDR-L3 having the amino acid sequence of SEQ ID NO: 33, a CDR-H1 having the amino acid sequence of SEQ ID NO: 38, a CDR-H2 having the amino acid sequence of SEQ ID NO: 39, and a CDR-H3 having the amino acid sequence of SEQ ID NO: 40. In some embodiments, the CDR-L1, CDR-L2, CDR-L3, CDR-H1, CDR-H2, and CDR-H3 are determined by Kabat numbering.

In some embodiments, the antibody or antigen binding fragment thereof comprises a light chain variable (V_{L}) region comprising the amino acid sequence of SEQ ID NO: 41 and a heavy chain variable (V_{H}) region comprising the amino acid sequence of SEQ ID NO: 42. In some embodiments, the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 41 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 42. In some embodiments, the antibody or antigen binding fragment thereof comprises a V_{L} region comprising the amino acid sequence of SEQ ID NO: 41, optionally with up to 10 (e.g., 1, 2, 3, 4, 5, 6, 7, 8, 9 or 10) substitutions (e.g., conservative substitutions) and a V_{H} region comprising the amino acid sequence of SEQ ID NO: 42, optionally with up to 10 (e.g., 1, 2, 3, 4, 5, 6, 7, 8, 9, or 10) substitutions (e.g., conservative substitutions).

Also provided herein are anti-BCMA antibodies or antigen binding fragments thereof comprising: a CDR-L1 having the amino acid sequence of SEQ ID NO: 43, a CDR-L2 having the amino acid sequence LGS, a CDR-L3 having the amino acid sequence of SEQ ID NO: 44, a CDR-H1 having the amino acid sequence of SEQ ID NO: 10, a CDR-H2 having the amino acid sequence of SEQ ID NO: 45, and a CDR-H3 having the amino acid sequence of SEQ ID NO: 46. In some embodiments, the CDR-L1, CDR-L2, CDR-L3, CDR-H1, CDR-H2, and CDR-H3 are determined by IMGT numbering.

Also provided herein are anti-BCMA antibodies or antigen binding fragments thereof comprising: a CDR-L1 having the amino acid sequence of SEQ ID NO: 47, a CDR-L2 having the amino acid sequence of SEQ ID NO: 48, a CDR-L3 having the amino acid sequence of SEQ ID NO: 44, a CDR-H1 having the amino acid sequence of SEQ ID NO: 49, a CDR-H2 having the amino acid sequence of SEQ ID NO: 50, and a CDR-H3 having the amino acid sequence of SEQ ID NO: 51. In some embodiments, the CDR-L1, CDR-L2, CDR-L3, CDR-H1, CDR-H2, and CDR-H3 are determined by Kabat numbering.

In some embodiments, the antibody or antigen binding fragment thereof comprises a light chain variable (V_{L}) region comprising the amino acid sequence of SEQ ID NO: 52 and a heavy chain variable (V_{H}) region comprising the amino acid sequence of SEQ ID NO: 53. In some embodiments, the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 52 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 53. In some embodiments, the antibody or antigen binding fragment thereof comprises a V_{L} region comprising the amino acid sequence of SEQ ID NO: 52, optionally with up to 10 (e.g., 1, 2, 3, 4, 5, 6, 7, 8, 9 or 10) substitutions (e.g., conservative substitutions) and a V_{H} region comprising the amino acid sequence of SEQ ID NO: 53, optionally with up to 10 (e.g., 1, 2, 3, 4, 5, 6, 7, 8, 9, or 10) substitutions (e.g., conservative substitutions).

Also provided herein are anti-BCMA antibodies or antigen binding fragments thereof comprising: a CDR-L1 having the amino acid sequence of SEQ ID NO: 43, a CDR-L2 having the amino acid sequence LGS, a CDR-L3 having the amino acid sequence of SEQ ID NO: 44, a CDR-H1 having the amino acid sequence of SEQ ID NO: 10, a CDR-H2 having the amino acid sequence of SEQ ID NO: 45, and a CDR-H3 having the amino acid sequence of SEQ ID NO: 46. In some embodiments, the CDR-L1, CDR-L2, CDR-L3, CDR-H1, CDR-H2, and CDR-H3 are determined by IMGT numbering.

Also provided herein are anti-BCMA antibodies or antigen binding fragments thereof comprising: a CDR-L1 having the amino acid sequence of SEQ ID NO: 47, a CDR-L2 having the amino acid sequence of SEQ ID NO: 48, a CDR-L3 having the amino acid sequence of SEQ ID NO: 44, a CDR-H1 having the amino acid sequence of SEQ ID NO: 54, a CDR-H2 having the amino acid sequence of SEQ ID NO: 50, and a CDR-H3 having the amino acid sequence of SEQ ID NO: 51. In some embodiments, the CDR-L1, CDR-L2, CDR-L3. CDR-H1, CDR-H2, and CDR-H3 are determined by Kabat numbering.

In some embodiments, the antibody or antigen binding fragment thereof comprises a light chain variable (V_{L}) region comprising the amino acid sequence of SEQ ID NO: 52 and a heavy chain variable (V_{H}) region comprising the amino acid sequence of SEQ ID NO: 55. In some embodiments, the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 52 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 55. In some embodiments, the antibody or antigen binding fragment thereof comprises a V_{L} region comprising the amino acid sequence of SEQ ID NO: 52, optionally with up to 10 (e.g., 1, 2, 3, 4, 5, 6, 7, 8, 9 or 10) substitutions (e.g., conservative substitutions) and a V_{H} region comprising the amino acid sequence of SEQ ID NO: 55, optionally with up to 10 (e.g., 1, 2, 3, 4, 5, 6, 7, 8, 9, or 10) substitutions (e.g., conservative substitutions).

Also provided herein are anti-BCMA antibodies or antigen binding fragments thereof comprising: a CDR-L1 having the amino acid sequence of SEQ ID NO: 7, a CDR-L2 having the amino acid sequence SEQ ID NO: 8, a CDR-L3 having the amino acid sequence of SEQ ID NO: 56, a CDR-H1 having the amino acid sequence of SEQ ID NO: 57, a CDR-H2 having the amino acid sequence of SEQ ID NO: 58, and a CDR-H3 having the amino acid sequence of SEQ ID NO: 59. In some embodiments, the CDR-L1, CDR-L2, CDR-L3, CDR-H1, CDR-H2, and CDR-H3 are determined by IMGT numbering.

Also provided herein are anti-BCMA antibodies or antigen binding fragments thereof comprising: a CDR-L1 having the amino acid sequence of SEQ ID NO: 13, a CDR-L2 having the amino acid sequence of SEQ ID NO: 14, a CDR-L3 having the amino acid sequence of SEQ ID NO: 56, a CDR-H1 having the amino acid sequence of SEQ ID NO: 60, a CDR-H2 having the amino acid sequence of SEQ ID NO: 61, and a CDR-H3 having the amino acid sequence of SEQ ID NO: 62. In some embodiments, the CDR-L1. CDR-L2, CDR-L3, CDR-H1, CDR-H2, and CDR-H3 are determined by Kabat numbering.

In some embodiments, the antibody or antigen binding fragment thereof comprises a light chain variable (V_{L}) region comprising the amino acid sequence of SEQ ID NO: 63 and a heavy chain variable (V_{H}) region comprising the amino acid sequence of SEQ ID NO: 64. In some embodiments, the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 63 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 64. In some embodiments, the antibody or antigen binding fragment thereof comprises a V_{L} region comprising the amino acid sequence of SEQ ID NO: 63, optionally with up to 10 (e.g., 1, 2, 3, 4, 5, 6, 7, 8, 9 or 10) substitutions (e.g., conservative substitutions) and a V_{H} region comprising the amino acid sequence of SEQ ID NO: 64, optionally with up to 10 (e.g., 1, 2, 3, 4, 5, 6, 7, 8, 9, or 10) substitutions (e.g., conservative substitutions).

In some embodiments, the antibody or antigen binding fragment thereof is a human antibody, a humanized antibody, or a chimeric antibody. In some embodiments, the antibody or antigen binding fragment thereof is an antigen binding fragment selected from the group consisting of a Fab, a Fab′, a F(ab′)2, a Fv, and a scFv. In some embodiments, the antigen binding fragment is a scFv. In some embodiments, the VL region is amino-terminal to the VH region. In some embodiments, the VL region is carboxy-terminal to the VH region. In some embodiments, the VL region is joined to the VH region via a flexible linker. In some embodiments, the flexible linker comprises the amino acid sequence set forth in SEQ ID NO: 66. In some embodiments, the scFv comprises the amino acid sequence set forth in SEQ ID NO: 68, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). In some embodiments, the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 68. In some embodiments, the scFv comprises the amino acid sequence set forth in SEQ ID NO: 69, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). In some embodiments, the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 69. In some embodiments, the scFv comprises the amino acid sequence set forth in SEQ ID NO: 70, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). In some embodiments, the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 70. In some embodiments, the scFv comprises the amino acid sequence set forth in SEQ ID NO: 71, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). In some embodiments, the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 71. In some embodiments, the scFv comprises the amino acid sequence set forth in SEQ ID NO: 72, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). In some embodiments, the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 72.

Also provided herein is an anti-BCMA chimeric antigen receptor comprising: an extracellular antigen binding domain comprising an anti-BCMA antibody or antigen binding fragment thereof, and an intracellular signaling region comprising an OX40L intracellular signaling domain. In some embodiments, the OX40L intracellular signaling domain comprises an amino acid sequence set forth in SEQ ID NO: 79, SEQ ID NO: 80, or SEQ ID NO: 81. In some embodiments, the OX40L intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 79, SEQ ID NO: 80, or SEQ ID NO: 81.

In some embodiments, the extracellular antigen binding domain comprising an anti-BCMA antibody or antigen binding fragment thereof comprises the anti-BCMA antibody or antigen binding fragment thereof of any one of the embodiments described herein.

Also provided herein is an anti-BCMA chimeric antigen receptor comprising an extracellular antigen binding domain comprising the anti-BCMA antibody or antigen binding fragment thereof of any one the embodiments described herein and an intracellular region.

In some embodiments, the anti-BCMA CAR specifically binds to a B-cell maturation antigen (BCMA) protein. In some embodiments, the BCMA protein comprises the amino acid sequence of SEQ ID NO: 1 or SEQ ID NO: 2.

In some embodiments, the CAR comprises a transmembrane region. In some embodiments, the transmembrane region comprises a CD28 transmembrane domain. In some embodiments, the CD28 transmembrane domain comprises the amino acid sequence set forth in SEQ ID NO: 77.

In some embodiments, the anti-BCMA CAR further comprises a hinge domain between the extracellular antigen binding domain and the transmembrane domain. In some embodiments, the hinge domain comprises at least a portion of a CD8α hinge domain. In some embodiments, the CD8α hinge domain comprises an amino acid sequence set forth in SEQ ID NO: 76. In some embodiments, the CD8α hinge domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 76.

In some embodiments, the intracellular signaling region further comprises a CD28 intracellular signaling domain. In some embodiments, the intracellular signaling region further comprises a CD3-zeta (CD3ζ) signaling domain. In some embodiments, the intracellular signaling region further comprises a CD28 intracellular signaling domain and a CD3-zeta signaling domain. In some embodiments, the CD28 intracellular signaling domain comprises an amino acid sequence set forth in SEQ ID NO: 78. In some embodiments, the CD28 intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 78. In some embodiments, the CD3-zeta intracellular signaling domain comprises an amino acid sequence set forth in SEQ ID NO: 82. In some embodiments, the CD3-zeta intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 82. In some embodiments, the intracellular signaling region comprises an amino acid sequence set forth in SEQ ID NO: 83. In some embodiments, the intracellular signaling region comprises an amino acid sequence having or having at least 85%, 86%, 87%, 88%, 89%, 90%, 91%, 92%, 93%, 94%, 95%, 96%, 97%, 98%, or 99% identity to SEQ ID NO: 83.

In some embodiments, the anti-BCMA CAR comprises the amino acid sequence set forth in any one of SEQ ID NO: 88, SEQ ID NO: 90, SEQ ID NO: 92, SEQ ID NO: 94, or SEQ ID NO: 96, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). In some embodiments, the anti-BCMA CAR comprises an amino acid sequence having or having at least 85%, 86%, 87%, 88%, 89%, 90%, 91%, 92%, 93%, 94%, 95%, 96%, 97%, 98%, or 99% identity an amino acid sequence set forth in any one of SEQ ID NO: 88, SEQ ID NO: 90, SEQ ID NO: 92, SEQ ID NO: 94, or SEQ ID NO: 96.

Also provided herein are polynucleotides comprising a nucleic acid encoding the anti-BCMA CAR of any one of the embodiments described herein.

In some embodiments, the nucleic acid encoding the anti-BCMA CAR comprises the nucleic acid sequence set forth in any one of SEQ ID NO: 89, SEQ ID NO: 91, SEQ ID NO: 93, SEQ ID NO: 95, or SEQ ID NO: 97.

In some embodiments, the polynucleotide further comprises a nucleic acid encoding an IL-15. In some embodiments, the IL-15 comprises the amino acid sequence set forth in SEQ ID NO: 86. In some embodiments, the IL-15 is encoded by a nucleic acid comprising SEQ ID NO: 87. In some embodiments, the polynucleotide encodes a polyprotein comprising the CAR and the IL-15. In some embodiments, the polynucleotide further comprises a nucleic acid encoding a sel—cleaving peptide, optionally a T2A self-cleaving peptide. In some embodiments, the CAR is joined to the IL-15 by the self-cleaving peptide. In some embodiments, the self-cleaving peptide is capable of inducing ribosomal skipping between the CAR and the IL-15. In some embodiments, the self-cleaving peptide comprises the sequence set forth in SEQ ID NO: 84. In some embodiments, the self-cleaving peptide is encoded by the sequence set forth in SEQ ID NO: 85.

In some embodiments, the polynucleotide further comprises a nucleic acid encoding a signal sequence. In some embodiments, the signal sequence comprises the amino acid sequence set forth in SEQ ID NO: 74. In some embodiments, the nucleic acid encoding the signal sequence comprises the nucleic acid sequence set forth in SEQ ID NO: 75. In some embodiments, the polynucleotide encodes a polyprotein comprising the amino acid sequence set forth in SEQ ID NO: 98, SEQ ID NO: 99, SEQ ID NO: 100, SEQ ID NO: 101, or SEQ ID NO: 102. In some embodiments, the polynucleotide comprises the nucleic acid sequence set forth in SEQ ID NO: 103, SEQ ID NO: 105. SEQ ID NO: 107, SEQ ID NO: 109, or SEQ ID NO: 111.

Also provided herein are vectors comprising the polynucleotide of any one of the embodiments disclosed herein. In some embodiments, the vector is a viral vector. In some embodiments, the viral vector is a retroviral vector or a lentiviral vector.

Also described herein are cells comprising the polynucleotide or the vector of any of the embodiments described herein. Also described herein are cells expressing the chimeric antigen receptor encoded by the polynucleotide or the vector of any of the embodiments described herein. Also described herein are cells expressing the chimeric antigen receptor and the IL-15 encoded by the polynucleotide or the vector of any of the embodiments described herein. In some embodiments, the cell is a lymphocyte. In some embodiments, the lymphocyte is a natural killer (NK) cell. In some embodiments, the lymphocyte is a T cell. In some embodiments, the cell is a human cell. In some embodiments, the cell is a primary cell obtained from a subject. In some embodiments, the cell is a primary cell obtained from cord blood. In some embodiments, the cell comprises a KIR-B haplotype. In some embodiments, the cell express CD16 having the V/V polymorphism at F158.

Also described herein are populations of cells comprising a plurality of the cells described above. In some embodiments, the population comprises expanded natural killer cells. In some embodiments, the expanded natural killer cells are expanded umbilical cord blood natural killer cells. In some embodiments, at least 70% of the NK cells are CD56+ and CD16+. In some embodiments, at least 85% of the NK cells are CD56+ and CD3−. In some embodiments, T % or less of the NK cells are CD3+, 1% or less of the NK cells are CD19+ and 1% or less of the NK cells are CD14+. In some embodiments, the population of expanded natural killer cells comprises at least 60%, e.g., at least 70%, at least 80%, at least 90% at least 95%, at least 99%, or 100% CD16+ cells. In some embodiments, the population of expanded natural killer cells comprises at least 60%, e.g., at least 70%, at least 80%, at least 90% at least 95%, at least 99%, or 100% NKG2D+ cells. In some embodiments, the population of expanded natural killer cells comprises at least 60%. e.g., at least 70%, at least 80%, at least 90% at least 95%, at least 99%, or 100% NKp46+ cells. In some embodiments, the population of expanded natural killer cells comprises at least 60%, e.g., at least 70%, at least 80%, at least 90% at least 95%, at least 99%, or 100% NKp30+ cells. In some embodiments, the population of expanded natural killer cells comprises at least 60%, e.g., at least 70%, at least 80%, at least 90% at least 95%, at least 99%, or 100% DNAM-1+ cells. In some embodiments, the population of expanded natural killer cells comprises at least 60%, e.g., at least 70%, at least 80%, at least 90% at least 95%, at least 99%, or 100% NKp44+ cells. In some embodiments, the population of expanded natural killer cells comprises less than 20%. e.g., 10% or less, 5% or less, 1% or less, 0.5% or less, or 0% CD3+ cells. In some embodiments, the population of expanded natural killer cells comprises less than 20% or less, e.g., 10% or less. 5% or less, 1% or less, 0.5% or less, or 0% CD14+ cells. In some embodiments, the population of expanded natural killer cells comprises less than 20% or less, e.g., 10% or less, 5% or less, 1% or less, 0.5% or less, or 0% CD19+ cells. In some embodiments, the population of expanded natural killer cells comprises less than 40% or less, 35% or less, 30% or less, 25% or less, or 20% or less, e.g., 10% or less, 5% or less, 1% or less, 0.5% or less, or 0% CD38+ cells. In some embodiments, the natural killer cells do not comprise a CD16 transgene. In some embodiments, the natural killer cells do not express an exogenous CD16 protein. In some embodiments, the expanded natural killer cells are derived from a single umbilical cord blood donor. In some embodiments, the umbilical cord blood is from a donor with the KIR-B haplotype and homozygous for the CD16 158V polymorphism.

In some embodiments, the population of NK cells is produced by a method comprising expanding the natural killer cells from umbilical cord blood at least 10,000 fold, e.g., 15,000 fold, 20,000 fold, 25,000 fold, 30,000 fold, 35,000 fold, 40,000 fold, 45,000 fold, 50,000 fold, 55,000 fold. 60.000 fold, 65,000 fold, or 70,000 fold. In some embodiments, the population of expanded natural killer cells is not enriched or sorted after expansion.

In some embodiments, the percentage of NK cells expressing CD16 in the population of expanded natural killer cells is the same or higher than the percentage of natural killer cells in the seed cells from umbilical cord blood. In some embodiments, the percentage of NK cells expressing NKG2D in the population of expanded natural killer cells is the same or higher than the percentage of natural killer cells in the seed cells from umbilical cord blood. In some embodiments, the percentage of NK cells expressing NKp30 in the population of expanded natural killer cells is the same or higher than the percentage of natural killer cells in the seed cells from umbilical cord blood. In some embodiments, the percentage of NK cells expressing NKp44 in the population of expanded natural killer cells is the same or higher than the percentage of natural killer cells in the seed cells from umbilical cord blood. In some embodiments, the percentage of NK cells expressing NKp46 in the population of expanded natural killer cells is the same or higher than the percentage of natural killer cells in the seed cells from umbilical cord blood. In some embodiments, the percentage of NK cells expressing DNAM-1 in the population of expanded natural killer cells is the same or higher than the percentage of natural killer cells in the seed cells from umbilical cord blood.

In some embodiments, the population of NK cells comprises at least 100 million expanded natural killer cells, e.g., 200 million, 250 million, 300 million, 400 million, 500 million, 600 million, 700 million, 750 million, 800 million, 900 million, 1 billion, 2 billion. 3 billion. 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, at least 10%, 20%, 30%, 40%, 50%, 60%, 70%, 80%, 85%, 90%; 95%, 96%, 97%, 98%, or 99% of the cells comprise the CAR of any one of the embodiments above, the polynucleotide of any one of the embodiments above, or the vector of any one of the embodiments above. In some embodiments, at least 10%, 20%, 30%, 40%, 50%, 60%. 70%, 80%, 85%, 90%: 95%, 96%, 97%, 98%, or 99% of the cells express the CAR of any one of the embodiments above, the polynucleotide of any one of the embodiments above, or the vector of any one of the embodiments above.

Also disclosed herein are pharmaceutical compositions comprising the cell of any one of the embodiments above or the population of cells of any one of the embodiments above. In some embodiments, the pharmaceutical composition further comprises a pharmaceutically acceptable excipient.

Also provided herein are frozen vials comprising the pharmaceutical compositions described above.

Also provided herein are methods of treatment comprising administering the cell of any of the embodiments described herein, the population of cells of any of the embodiments described herein, or the composition of any of the embodiments described herein to a subject having a disease or condition associated with BCMA.

Also provided herein are uses of the cell of any one of the embodiments described herein, the population of cells of any one the embodiments described herein, or the composition of any one the embodiments described herein in the manufacture of a medicament for treating a disease or condition associated with BCMA.

Also provided herein are uses of the cell of any of the embodiments described herein, the population of cells of any of the embodiments described herein, or the composition of any one of the embodiments described herein for treating a disease or condition associated with BCMA.

In some embodiments, the disease or condition associated with BCMA is cancer. In some embodiments, the cancer is a BCMA+ cancer. In some embodiments, the BCMA+ cancer is or comprises a hematopoietic neoplastic disorder expressing BCMA. In some embodiments, the cancer is or comprises neoplastic cells of hematopoietic origin. In some embodiments, the cancer arises from a lymphoid lineage, or a precursor cell thereof. In some embodiments, the cancer arises from a plasma cell, or a precursor cell thereof.

In some embodiments, the disease or condition associated with BCMA is a myeloma, smoldering myeloma, plasmacytoma, multiple myeloma, leukemia, or lymphoma. In some embodiments, the disease or condition associated with BCMA is multiple myeloma. In some embodiments, the multiple myeloma is a high-risk myeloma or a lenalidomide-refractory multiple myeloma. In some embodiments, the subject has relapsed after treatment with an anti-CD38 antibody. In some embodiments, the subject has experienced disease progression after treatment with autologous stem cell transplant or chimeric antigen receptor T-cell therapy (CAR-T).

In some embodiments, the cells are allogenic to the subject.

In some embodiments, the method or use further comprises administering an antibody targeted to CD38. In some embodiments, the antibody is daratumumab, isatuximab, or a biosimilar thereof. In some embodiments, the antibody is daratumumab. In some embodiments, the antibody is isatuximab.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a,” “an” and “the” include plural references unless the context clearly dictates otherwise. For example, the term “a sample” includes a plurality of samples, including mixtures thereof.

The terms “determining,” “measuring.” “evaluating.” “assessing,” “assaying.” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows epitope footprints of anti-BCMA antibodies

FIG. 2 shows crystal structures of BCMA complexes.

FIG. 3 shows the BCMA-APRIL complex crystal structure.

FIG. 4 shows CAR-NK cell expression.

FIG. 5 shows CAR-NK cell potency.

FIG. 6 shows CAR-NK cell long term cytotoxicity.

FIG. 7 shows CAR-NK cell long term cytotoxicity.

FIG. 8 shows anti-BCMA CAR NK cells tolerance toward soluble BCMA (sBCMA).

FIG. 9 shows long-term cytotoxicity of anti-BCMA CAR NK cells in the presence of daratumumab.

FIG. 10 shows expression of markers of degranulation.

FIG. 11 shows production of effector cytokines.

FIG. 12 shows levels of tolerance toward soluble BCMA (sBCMA).

FIG. 13 shows levels of tolerance toward soluble BCMA (sBCMA).

### DETAILED DESCRIPTION

Described herein are, among other things, BCMA-binding agents. e.g., anti-BCMA antibodies and antigen-binding fragments thereof, chimeric antigen receptors comprising the same, and natural killer cells expressing the chimeric antigen receptors. Also provided herein are pharmaceutical compositions comprising the anti-BCMA antibodies and antigen-binding fragments thereof, chimeric antigen receptors comprising the same, or natural killer cells. Also provided herein are methods of treatment. e.g., of BCMA+ cancers, comprising administering the anti-BCMA antibodies and antigen-binding fragments thereof, chimeric antigen receptors comprising the same, or natural killer cells described herein.

### I. BCMA

B cell maturation antigen (BCMA, also known as CD269 and TNF receptor superfamily 17 (TNFRSF17)), is a receptor for TNFSF13B/BLyS/BAFF and TNFSF13/APRIL. It promotes B-cell survival and plays a role in the regulation of humoral immunity. It also activates NF-kappa and JNK. Human BCMA (UniProt Q02223, TNR17_HUMAN) has two isoforms, the amino acid sequences of which are set forth below.

>sp/Q02223|TNR17_HUMAN Tumor necrosis factor
receptor superfamily member 17
OS = Homo sapiens
OX = 9606
GN = TNFRSF17
PE = 1 SV = 2
SEQ ID NO: 1
MLQMAGQCSQNEYFDSLLHACIPCQLRCSSNTPPLTCQRYCNASV
INSVKGTNAILWTCLGLSLIISLAVFVLMFLLRKINSEPLKDEFK
NTGSGLLGMANIDLEKSRTGDEIILPRGLEYTVEECTCEDCIKSK
PKVDSDHCFPLPAMEEGATILVTTKTNDYCKSLPAALSATEIEKS
ISAR
>sp|Q02223-2|TNR17_HUMAN Isoform 2 of Tumor
necrosis factor receptor superfamily member 17
OS = Homo sapiens
OX = 9606 GN = TNFRSF17
SEQ ID NO: 2
MLQMAGQCSQNEYFDSLLHACIPCQLRCSSNTPPLTCQRYCNARS
GLLGMANIDLEKSRTGDEIILPRGLEYTVEECTCEDCIKSKPKVD
SDHCFPLPAMEEGATILVTTKTNDYCKSLPAALSATEIEKSISAR

BCMA is a validated target in Multiple Myeloma (MM). BCMA is highly and uniformly expression on MM cells, with expression in normal tissue limited to plasma cells. BCMA is shed from malignant cells through the action of γ-secretase and elevated levels of soluble BCMA (sBCMA) in MM patients correlate with poor clinical outcomes. sBCMA level in MM patients is 5 nM (~100 ng/mL, median), but potentially as high as 25 nM (~500 ng/mL). The soluble BCMA may interfere with the myeloma-targeting capacities of BCMA-specific immunotherapies. See, e.g., Panowski et al., “Preclinical Efficacy and Safety Comparison of CD3 Bispecific and ADC Modalities Targeting BCMA for the Treatment of Multiple Myeloma,” Molecular Cancer Therapeutics 18(11):2008-20 (2019).

Described herein are antibodies and antigen-binding fragments thereof that bind to BCMA, preferably human BCMA (e.g., SEQ ID NO: 1 and/or SEQ ID NO: 2). In some cases, the antibodies and antigen binding fragments thereof described herein specifically binds to BCMA. In some cases, the antibodies and antigen binding fragments thereof have a higher affinity to membrane bound BCMA than to soluble BCMA.

### II. ANTI-BCMA ANTIBODIES AND ANTIGEN BINDING FRAGMENTS THEREOF

Described herein are BCMA-binding agents. e.g., anti-BCMA antibodies and antigen binding fragments thereof.

Described herein are antibodies and antigen binding fragments thereof that bind to BCMA. The term “antibody” refers to an immunoglobulin molecule or immunologically active portion thereof, i.e., an antigen-binding portion.

The term “monoclonal antibody” as used herein refers to an antibody obtained from a population of substantially homogeneous antibodies, e.g., the individual antibodies comprising the population are identical except for possible naturally-occurring mutations that may be present in minor amounts. An antibody can be monoclonal. An antibody can be a human or humanized antibody. The term “monoclonal antibody” encompasses intact and full-length monoclonal antibodies as well as antibody fragments (e.g., Fab, Fab′, F(ab′)₂, Fv), single chain antibodies (e.g., scFv), fusion proteins comprising an antibody fragment, and any other modified immunoglobulin molecule comprising at least one antigen-binding site. Furthermore. “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR residues are replaced by residues from corresponding CDRs from a nonhuman antibody (e.g., mouse, rat, rabbit, or nonhuman primate), wherein the nonhuman antibody has the desired specificity, affinity, and/or activity. In some embodiments, one or more framework region residues of the human heavy chain or light chain variable regions are replaced by corresponding residues from nonhuman antibody. Furthermore, humanized antibodies can comprise residues that are not found in the human antibody or in the nonhuman antibody. In some embodiments, these modifications are made to further refine and/or optimize antibody characteristics. In some embodiments, the humanized antibody comprises at least a portion of an immunoglobulin constant region (e.g., CH1, CH2, CH3, Fc), typically that of a human immunoglobulin.

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

“Antibody fragments” can include a portion of an intact antibody, preferably the antigen binding or variable region of the intact antibody. Examples of antibody fragments include Fab, Fab′, F(ab′)2, and Fv fragments; diabodies; linear antibodies; single-chain antibody molecules; and multispecific antibodies formed from antibody fragments.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of software bioinformatic tools available on the internet. X-ray crystallography may be used to characterize an epitope on a target protein by analyzing the amino acid residue interactions of an antigen/antibody complex.

“Fv” includes the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgB1, IgG2, IgG3, IgG4, IgA, and IgA2. “Single-chain Fv” or “sFv” antibody fragments comprise the VH and VL domains of antibody, wherein these domains are present in a single polypeptide chain. In some cases, the Fv polypeptide further comprises a polypeptide linker between the VH and VL domains which enables the sFv to form the desired structure for antigen binding.

In various embodiments, the antibody or antigen binding fragment thereof comprises a human or humanized antibody. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. Methods for humanizing non-human antibodies are well known in the art.

The BCMA antibodies described herein can be affinity matured, for example using selection and/or mutagenesis methods known in the art. In general, an “affinity matured” antibody is one with one or more alterations in one or more hyper variable regions thereof which result in an improvement in the affinity of the antibody for antigen, compared to a parent antibody which does not possess those alteration(s). In one embodiment, an affinity matured antibody has nanomolar or even picomolar affinities for the target antigen. Preferred affinity matured antibodies have an affinity that is five times, more preferably 10 times, even more preferably 20 or 30 times greater than the starting antibody (generally murine, humanized or human) from which the matured antibody is prepared.

An antibody that “binds to,” “specifically binds to,” or is “specific for” a particular polypeptide or an epitope on a particular polypeptide is one that binds to that particular polypeptide or epitope on a particular polypeptide without substantially binding to any other polypeptide or polypeptide epitope. The term “specifically binds” as used herein refers to a BCMA agent (e.g., an anti-BCMA antibody) that interacts more frequently, more rapidly, with greater duration, with greater affinity, or with some combination of the above to a particular antigen, epitope, protein, or target molecule than with alternative substances. A binding agent (e.g. antibody) that specifically binds an antigen can be identified, for example, by immunoassays, ELISAs, Surface Plasmon Resonance (SPR) assays (e.g., Biacore), or other techniques known to those of skill in the art. As such, described herein are functional equivalents to the specific anti-BCMA antibodies described. In some cases, the BCMA antibody may be cross reactive with various similar BCMA proteins (e.g., with highest affinity for one, such as membrane bound BCMA, and lower affinity for others, such as soluble BCMA). A binding agent that specifically binds an antigen binds the target antigen with a higher affinity than its affinity for a different antigen. The different antigen can be a related antigen. In some embodiments, a binding agent that specifically binds an antigen binds the target antigen with an affinity that is at least 20 times greater than its affinity for a different antigen, e.g., at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, a binding agent that specifically binds a particular antigen binds a different antigen at such a low affinity that binding cannot be detected using an assay described herein or otherwise known in the art. In some embodiments, affinity is measured using SPR technology, e.g., in a Biacore system or other system known to those of skill in the art.

The terms “identical” or “percent identity” in the context of two or more polypeptides (e.g., two anti-BCMA antibodies), refer to two or more sequences or subsequences that are the same or have a specified percentage of amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm. Such homology is well-represented in the art via local alignment tools and/or algorithms, and may include pairwise alignment, multiple sequence alignment methods, structural alignment methods, and/or phylogenetic analysis methods. Where sequences differ in conservative substitutions, the percent sequence identity may be, but not necessarily is, adjusted upwards to correct for the conservative nature of the substitution. Means for making this adjustment are well known to those of skill in the art. Typically, but not necessarily, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1.

In some embodiments, two polypeptides (e.g., antibodies or antibody domains (e.g., VL, CL, VH, CH1, CH2, CH3 domains) thereof) of the disclosure are substantially identical, meaning they have at least 70%, at least 75%, at least 80%, at least 85%, at least 90%, and in some embodiments at least 95%, 96%, 97%, 98%, 99% amino acid residue identity, when compared and aligned for maximum correspondence, as measured using a sequence comparison algorithm or by visual inspection. In some embodiments, percent identity exists over a region of the sequences that is at least about 10, at least about 20, at least about 20-40, at least about 40-60 amino acid residues, at least about 60-80 nucleotides or amino acid residues in length or any integral value there between. In some embodiments, percent identity exists over a longer region than 60-80 amino acid residues, such as at least about 80-100 amino acid residues, and in some embodiments the sequences are substantially identical over the full length of the sequences being compared, for example, an amino acid sequence.

Also contemplated herein are polypeptides (e.g., antibodies or antibody domains (e.g., VL, CL, VH, CH1, CH2, CH3 domains) thereof) of the disclosure with substitutions relative to a given SEQ ID NO. For example, up to 10 (i.e., 1, 2, 3, 4, 5, 6, 7, 8, 9, or 10) substitutions or up to 20 (i.e., 1, 2, 3, 4, 5, 6, 7, 8, 9, 10, 11, 12, 13, 14, 15, 16, 17, 18, 19, or 20) substitutions. In some cases, the substitutions are conservative substitutions. In some cases, the substitutions are not at critical residues (e.g., as described herein).

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

The terms “conservative sequence modifications” or “conservative substitutions” as used herein may refer to amino acid modifications to a target epitope or antibodies and antigen-binding portions thereof of the disclosure that does not significantly affect or alter the binding characteristics of the anti-BCMA antibodies. “Conservative substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Methods of identifying amino acid conservative substitutions that do not eliminate binding are well-known in the art.

In some embodiments, a BCMA-binding agent is an antibody, e.g., a full length antibody comprising an Fc domain including at least one heavy chain. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, the antibody is an antibody fragment comprising an antigen-binding site. In some embodiments, the antibody is a scFv. In some embodiments, the antibody is a disulfide-linked scFv. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody. In some embodiments, the antibody is isolated. In some embodiments, the antibody is substantially pure. In some embodiments, a BCMA-binding agent is a polyclonal antibody. Polyclonal antibodies can be prepared by any method known to those of skill in the art. In some embodiments, polyclonal antibodies are produced by immunizing an animal (e.g., a rabbit, rat, mouse, goat, donkey) with an antigen of interest (e.g., a purified peptide fragment, a recombinant protein, or a fusion protein) using multiple subcutaneous or intraperitoneal injections. In some embodiments, the antigen is conjugated to a carrier such as keyhole limpet hemocyanin (KLH), serum albumin, bovine thyroglobulin, or soybean trypsin inhibitor. The antigen (with or without a carrier protein) is diluted in sterile saline and usually combined with an adjuvant (e.g., Complete or Incomplete Freund's Adjuvant) to form a stable emulsion. After a period of time, polyclonal antibodies are recovered from the immunized animal (e.g., from blood or ascites). In some embodiments, the polyclonal antibodies are purified from serum or ascites according to standard methods in the art including, but not limited to, affinity chromatography, ion-exchange chromatography, gel electrophoresis, and/or dialysis.

In some embodiments, a BCMA-binding agent is a monoclonal antibody. Monoclonal antibodies can be prepared by any method known to those of skill in the art. In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a mouse protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In some embodiments, recombinant monoclonal antibodies are isolated from phage display libraries expressing variable domains or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are substituted for constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and affinity of a monoclonal antibody.

In some embodiments, a BCMA-binding agent is a humanized antibody. Various methods for generating humanized antibodies are known in the art. In some embodiments, a humanized antibody comprises one or more amino acid residues that have been introduced into it from a source that is non-human. In some embodiments, humanization is performed by substituting one or more non-human CDR sequences for the corresponding CDR sequences of a human antibody. In some embodiments, the humanized antibodies are constructed by substituting all six CDRs of anon-human antibody (e.g., a mouse antibody) for the corresponding CDRs of a human antibody.

The choice of which human heavy chain variable region and/or light chain variable region is used for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, a method called “superhumanization” which is described as the direct transfer of CDRs to a human germline framework, a method termed Human String Content (HSC) which is based on a metric of “antibody humanness”, methods based on generation of large libraries of humanized variants (including phage, ribosomal, and yeast display libraries), and methods based on framework region shuffling.

In some embodiments, a BCMA-binding agent is a human antibody. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

In some embodiments, a BCMA-binding agent is a scFv antibody. ScFvs are molecules that comprise a variable heavy chain region and a variable light chain region linked to form a single polypeptide. ScFvs can be produced using recombinant technologies known in the art. In some embodiments, a scFv comprises a polypeptide linker between the heavy chain variable region and the light chain variable region. In some embodiments, the scFv comprises an orientation of (from N- to C-terminus) (i) heavy chain variable region, (ii) linker, and (iii) light chain variable region. In some embodiments, the scFv comprises an orientation (from N- to C-terminus) of (i) light chain variable region, (ii) linker, and (iii) heavy chain variable region. In some embodiments, the scFv is a disulfide-linked scFv (dsscFv), which is a scFv comprising an engineered disulfide bond between the light chain variable region and heavy chain variable region of the scFv. In some embodiments, the scFv (e.g., dsscFv) is attached (either directly or indirectly) to a half-life extending moiety such as, e.g., an Fc molecule, a CH3 domain of an immunoglobulin (e.g., CH3 of IgG1), polyethylene glycol (PEG) or a PEG mimetic, XTEN, serum albumin (e.g., human serum albumin), polysicalic acid, N-(2-hydroxypropyl)methacrylamide, or dextran, or is modified by, e.g., hyperglycosylation, to extend the half-life of the scFv (e.g., dsscFv).

A variety of suitable linkers are known to those of skill in the art and are not limited by any specific sequences disclosed herein. In some embodiments, the polypeptide linker is comprised of naturally, or non-naturally, occurring amino acids. In some embodiments, the linker comprises amino acids that allow for flexibility. In some embodiments, the linker comprises amino acids that allow for suitable solubility. In some embodiments, the linker comprises glycine amino acids. In some embodiments, the linker comprises glycine and serine amino acids. In certain embodiments, the linker comprises one or more sets of glycine/serine repeats. In some embodiments, the polypeptide linker is (GGGGS)n wherein n=1-4. In some embodiments, the polypeptide linker is selected from the group consisting of GGGGS, GGGGSGGGGS (SEQ ID NO: 65), GGGGSGGGGSGGGGS (SEQ ID NO: 66), and GGGGSGGGGSGGGGSGGGGS (SEQ ID NO: 67).

In some embodiments, a BCMA-binding agent is a Fv. A Fv comprises a heavy chain variable region and a light chain variable region. In some embodiments, the Fv is attached (either directly or indirectly) to a half-life extending moiety such as, e.g., an Fc molecule, a CH3 domain of an IgG (e.g., CH3 of IgG1), PEG or a PEG mimetic, XTEN, serum albumin (e.g., human serum albumin), polysicalic acid, N-(2-hydroxypropyl)methacrylamide, or dextran, or is modified, e.g., by hyperglycosylation, to extend the half-life of the Fv.

In some embodiments, a BCMA-binding agent is a Fab. A Fab is one of the molecules that result from digestion of an immunoglobulin antibody with papain. Fabs are monovalent molecules that comprise a light chain, a heavy chain variable region, a CH1 region, and, optionally, a heavy chain constant region hinge region or a portion thereof. Fabs can be produced using recombinant technologies known in the art. In some embodiments, a Fab comprises a polypeptide linker between the light chain constant region and the heavy chain variable region. In some embodiments, a Fab comprises a polypeptide linker between the heavy chain constant region and the light chain variable region. A variety of suitable linkers are known to those of skill in the art and are not limited by any specific sequences disclosed herein. In certain embodiments, the linker is a linker described herein. In some embodiments, the Fab is attached (either directly or indirectly) to a half-life extending moiety such as, e.g., an Fc molecule, a CH3 domain of an IgG (e.g., CH3 of IgG1), PEG or a PEG mimetic, XTEN, serum albumin (e.g., human serum albumin), polysicalic acid, N-(2-hydroxypropyl)methacrylamide, or dextran, or is modified. e.g., by hyperglycosylation, to extend the half-life of the Fab.

In some embodiments, a Fab comprises a disulfide bond formed between the heavy chain variable region and the light chain variable region. In some embodiments, a Fab comprises a disulfide bond that increases stability of the Fab molecule. In some embodiments, a Fab comprises a disulfide bond that increases thermostability of the Fab molecule.

In some embodiments, a BCMA-binding agent is a F(ab′)₂. A F(ab′)₂ is one of the molecules that results from digestion of an immunoglobulin antibody with pepsin. A F(ab′)₂ is a divalent molecule that comprises a first light chain in association with a first polypeptide comprising a first heavy chain variable region, a first CH1, and a first hinge region, and a second light chain in association with a second polypeptide comprising a second heavy chain variable region, a second CH1, and a second hinge region, wherein the first hinge region is linked to the second hinge region via at least one disulfide bond. F(ab′)₂s can be produced using recombinant technologies known in the art. In some embodiments, the F(ab′)₂ is attached (either directly or indirectly) to a half-life extending moiety such as, e.g., a CH3 domain of an IgG (e.g., CH3 of IgG1), PEG or a PEG mimetic, XTEN, serum albumin (e.g., human serum albumin), polysicalic acid, N-(2-hydroxypropyl)methacrylamide, or dextran, or is modified, e.g., by hyperglycosylation, to extend the half-life of the F(ab′)₂.

In some embodiments, a F(ab′)₂ comprises a disulfide bond formed between the heavy chain variable region and the light chain variable region. In some embodiments, a F(ab′)₂ comprises a disulfide bond that increases stability of the F(ab′)₂ molecule. In some embodiments, a F(ab′)₂ comprises a disulfide bond that increases thermostability of the F(ab′)₂ molecule.

In some embodiments, a BCMA-binding agent is a F(ab′). A F(ab′) is a molecule that results from treatment of a F(ab′)₂ with beta-mercaptoethanol. A F(ab′) is a monovalent molecule that comprises a light chain in association with a polypeptide comprising a heavy chain variable region, a CH1, and a hinge region. In some embodiments, the F(ab′) is attached (either directly or indirectly) to a half-life extending moiety such as, e.g., an Fc molecule, a CH3 domain of an IgG (e.g., CH3 of IgG1). PEG or a PEG mimetic, XTEN, serum albumin (e.g., human serum albumin), polysicalic acid. N-(2-hydroxypropyl)methacrylamide, or dextran, or is modified, e.g., by hyperglycosylation, to extend the half-life of the F(ab′).

In some embodiments, a F(ab′) comprises a disulfide bond formed between the heavy chain variable region and the light chain variable region. In some embodiments, a F(ab′) comprises a disulfide bond that increases stability of the F(ab′) molecule. In some embodiments, a F(ab′) comprises a disulfide bond that increases thermostability of the F(ab′) molecule.

In some embodiments, a BCMA-binding agent is a bispecific antibody. Bispecific antibodies are capable of recognizing and binding at least two different antigens or epitopes. The different epitopes can either be within the same molecule (e.g., two epitopes on BCMA) or on different molecules (e.g., one epitope on BCMA and one epitope on a different target). In some embodiments, a bispecific antibody has enhanced potency as compared to an individual antibody or to a combination of more than one antibody. In some embodiments, a bispecific antibody has reduced toxicity as compared to an individual antibody or to a combination of more than one antibody. It is known to those of skill in the art that any therapeutic agent may have unique pharmacokinetics (PK) (e.g., circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area (e.g., tissue) in a subject (e.g., a human). In some embodiments, a bispecific antibody has the ability to concentrate the actions of two agents to a common target (e.g., a specific cell type). In some embodiments, a bispecific antibody has the ability to target the actions of two agents to more than one biological pathway or function. In some embodiments, a bispecific antibody has the ability to target two different cells and bring them closer together.

In some embodiments, a bispecific antibody has decreased toxicity and/or side effects. In some embodiments, a bispecific antibody has decreased toxicity and/or side effects as compared to a mixture of the two individual antibodies or the antibodies as single agents. In some embodiments, a bispecific antibody has an increased therapeutic index. In some embodiments, a bispecific antibody has an increased therapeutic index as compared to a mixture of the two individual antibodies or the antibodies as single agents.

Several techniques for making bispecific antibodies are known by those skilled in the art. In some embodiments, the bispecific antibodies comprise heavy chain constant regions with modifications in the amino acids that are part of the interface between the two heavy chains. These modifications are made to enhance heterodimer formation and generally reduce or eliminate homodimer formation. In some embodiments, the bispecific antibodies are generated using a knobs-into-holes (KIH) strategy. In some embodiments, the bispecific antibodies comprise variant hinge regions incapable of forming disulfide linkages between identical heavy chains (e.g., reduce homodimer formation). In some embodiments, the bispecific antibodies comprise heavy chains with changes in amino acids that result in altered electrostatic interactions. In some embodiments, the bispecific antibodies comprise heavy chains with changes in amino acids that result in altered hydrophobic/hydrophilic interactions.

Bispecific antibodies can be intact antibodies or antibody fragments comprising antigen-binding sites.

BCMA-binding agents with more than two valencies are also contemplated. In some embodiments, trispecific or tetraspecific antibodies are generated.

In some embodiments, a BCMA-binding agent is an anti-BCMA antibody that comprises one, two, three, four, five, and/or six CDRs of any one of the antibodies described herein.

In some cases, the BCMA-binding agent binds to one or more of L2, E12, L17, I22, P23, L26, R27, or N31 of SEQ ID NO: 73. In some cases, the BCMA-binding agent binds to L2 of SEQ ID NO: 73. In some cases, the BCMA-binding agent binds to E12, I22, P23, and L26 of SEQ ID NO: 73. In some cases, the BCMA-binding agent binds to N31 of SEQ ID NO: 73. In some cases, the BCMA-binding agent binds to R27 and N31 of SEQ ID NO: 73. In some cases, the BCMA-binding agent binds to L17 and N31 of SEQ ID NO: 73.

In some cases, the anti-BCMA antibody or antigen binding fragment thereof comprises CDRL1, CDRL2, CDRL3, CDRHI1, CDRH2, and CDRH3 sequences of any one of the antibodies described in Table 3. Table 4, Table 5. Table 6, Table 7, or Table 8. Methods of determining and defining CDRs are well known in the art. Such methods include IMGT and Kabat.

Provided herein is an antibody or antigen binding fragment thereof comprising the amino acid sequences of SEQ ID NO: 7. SEQ ID NO: 8, and SEQ ID NO: 9. Also provided herein is an antibody or antigen binding fragment thereof comprising the amino acid sequences of SEQ ID NO: 10, SEQ ID NO: 11, and SEQ ID NO: 12. Also provided herein is an antibody or antigen binding fragment thereof comprising the amino acid sequences of SEQ ID NO: 7, SEQ ID NO: 8, SEQ ID NO: 9, SEQ ID NO: 10, SEQ ID NO: 11, and SEQ ID NO: 12.

Provided herein is an antibody or antigen binding fragment thereof comprising the amino acid sequences of SEQ ID NO: 13, SEQ ID NO: 14, and SEQ ID NO: 9. Also provided herein is an antibody or antigen binding fragment thereof comprising the amino acid sequences of SEQ ID NO: 15, SEQ ID NO: 16, and SEQ ID NO: 17. Also provided herein is an antibody or antigen binding fragment thereof comprising the amino acid sequences of SEQ ID NO: 13, SEQ ID NO: 14, SEQ ID NO: 9, SEQ ID NO: 15, SEQ ID NO: 16, and SEQ ID NO: 17.

Provided herein is an antibody or antigen binding fragment thereof comprising the amino acid sequences of SEQ ID NO: 20, WAS, and SEQ ID NO: 21. Also provided herein is an antibody or antigen binding fragment thereof comprising the amino acid sequences of SEQ ID NO: 22, SEQ ID NO: 23, and SEQ ID NO: 24. Also provided herein is an antibody or antigen binding fragment thereof comprising the amino acid sequences of SEQ ID NO: 20, WAS, SEQ ID NO: 21, SEQ ID NO: 22, SEQ ID NO: 23, and SEQ ID NO: 24.

Provided herein is an antibody or antigen binding fragment thereof comprising the amino acid sequences of SEQ ID NO: 25, SEQ ID NO: 26, and SEQ ID NO: 21. Also provided herein is an antibody or antigen binding fragment thereof comprising the amino acid sequences of SEQ ID NO: 27, SEQ ID NO: 28, and SEQ ID NO: 29. Also provided herein is an antibody or antigen binding fragment thereof comprising the amino acid sequences of SEQ ID NO: 25, SEQ ID NO: 26, SEQ ID NO: 21, SEQ ID NO: 27, SEQ ID NO: 28, and SEQ ID NO: 29.

Provided herein is an antibody or antigen binding fragment thereof comprising the amino acid sequences of SEQ ID NO: 32, SEQ ID NO: 8, and SEQ ID NO: 33. Also provided herein is an antibody or antigen binding fragment thereof comprising the amino acid sequences of SEQ ID NO: 34, SEQ ID NO: 35, and SEQ ID NO: 36. Also provided herein is an antibody or antigen binding fragment thereof comprising the amino acid sequences of SEQ ID NO: 32, SEQ ID NO: 8, SEQ ID NO: 33, SEQ ID NO: 34, SEQ ID NO: 35, and SEQ ID NO: 36.

Provided herein is an antibody or antigen binding fragment thereof comprising the amino acid sequences of SEQ ID NO: 37, SEQ ID NO: 14, and SEQ ID NO: 33. Also provided herein is an antibody or antigen binding fragment thereof comprising the amino acid sequences of SEQ ID NO: 38, SEQ ID NO: 39, and SEQ ID NO: 40. Also provided herein is an antibody or antigen binding fragment thereof comprising the amino acid sequences of SEQ ID NO: 37, SEQ ID NO: 14, SEQ ID NO: 33, SEQ ID NO: 38, SEQ ID NO: 39, and SEQ ID NO: 40.

Provided herein is an antibody or antigen binding fragment thereof comprising the amino acid sequences of SEQ ID NO: 43, LGS, and SEQ ID NO: 44. Also provided herein is an antibody or antigen binding fragment thereof comprising the amino acid sequences of SEQ ID NO: 10, SEQ ID NO: 45, and SEQ ID NO: 46. Also provided herein is an antibody or antigen binding fragment thereof comprising the amino acid sequences of SEQ ID NO: 43, LGS. SEQ ID NO: 44, SEQ ID NO: 10, SEQ ID NO: 45, and SEQ ID NO: 46.

Provided herein is an antibody or antigen binding fragment thereof comprising the amino acid sequences of SEQ ID NO: 47, SEQ ID NO: 48, and SEQ ID NO: 44. Also provided herein is an antibody or antigen binding fragment thereof comprising the amino acid sequences of SEQ ID NO: 49, SEQ ID NO: 50, and SEQ ID NO: 51. Also provided herein is an antibody or antigen binding fragment thereof comprising the amino acid sequences of SEQ ID NO: 47, SEQ ID NO: 48, SEQ ID NO: 44, SEQ ID NO: 49, SEQ ID NO: 50, and SEQ ID NO: 51.

Provided herein is an antibody or antigen binding fragment thereof comprising the amino acid sequences of SEQ ID NO: 43, LGS, and SEQ ID NO: 44. Also provided herein is an antibody or antigen binding fragment thereof comprising the amino acid sequences of SEQ ID NO: 10, SEQ ID NO: 45, and SEQ ID NO: 46. Also provided herein is an antibody or antigen binding fragment thereof comprising the amino acid sequences of SEQ ID NO: 43, LGS, SEQ ID NO: 44, SEQ ID NO: 10, SEQ ID NO: 45, and SEQ ID NO: 46.

Provided herein is an antibody or antigen binding fragment thereof comprising the amino acid sequences of SEQ ID NO: 47, SEQ ID NO: 48, and SEQ ID NO: 44. Also provided herein is an antibody or antigen binding fragment thereof comprising the amino acid sequences of SEQ ID NO: 54, SEQ ID NO: 50, and SEQ ID NO: 51. Also provided herein is an antibody or antigen binding fragment thereof comprising the amino acid sequences of SEQ ID NO: 47, SEQ ID NO: 48, SEQ ID NO: 44, SEQ ID NO: 54, SEQ ID NO: 50, and SEQ ID NO: 51.

Provided herein is an antibody or antigen binding fragment thereof comprising the amino acid sequences of SEQ ID NO: 7. SEQ ID NO: 8, and SEQ ID NO: 56. Also provided herein is an antibody or antigen binding fragment thereof comprising the amino acid sequences of SEQ ID NO: 57, SEQ ID NO: 58, and SEQ ID NO: 59. Also provided herein is an antibody or antigen binding fragment thereof comprising the amino acid sequences of SEQ ID NO: 7, SEQ ID NO: 8, SEQ ID NO: 56, SEQ ID NO: 57, SEQ ID NO: 58, and SEQ ID NO: 59.

Provided herein is an antibody or antigen binding fragment thereof comprising the amino acid sequences of SEQ ID NO: 13, SEQ ID NO: 14, and SEQ ID NO: 56. Also provided herein is an antibody or antigen binding fragment thereof comprising the amino acid sequences of SEQ ID NO: 60, SEQ ID NO: 61, and SEQ ID NO: 62. Also provided herein is an antibody or antigen binding fragment thereof comprising the amino acid sequences of SEQ ID NO: 13, SEQ ID NO: 14, SEQ ID NO: 56, SEQ ID NO: 60, SEQ ID NO: 61, and SEQ ID NO: 62.

In some cases, the antibody or antigen binding fragment thereof comprises the VL and VH sequences of any one of the antibodies described in Table 3, Table 4, Table 5, Table 6, Table 7, or Table 8, optionally with up to 20 (e.g., 1, 2, 3, 4, 5, 6, 7, 8, 9, 10, 11, 12, 13, 14, 15, 16, 17, 18, 19, or 20) substitutions (e.g, conservative substitutions).

Provided herein is an antibody or antigen binding fragment thereof comprising the amino acid sequence of SEQ ID NO: 18, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequence of SEQ ID NO: 19, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequences of SEQ ID NO: 18, optionally with up to 10 (e.g., 1, 2, 3, 4, 5, 6, 7, 8, 9, or 10) substitutions (e.g., conservative substitutions) and SEQ ID NO: 19, optionally with up to 10 (e.g., 1, 2, 3, 4, 5, 6, 7, 8, 9, or 10) substitutions (e.g., conservative substitutions).

Provided herein is an antibody or antigen binding fragment thereof comprising the amino acid sequence of SEQ ID NO: 30, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequence of SEQ ID NO: 31, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequences of SEQ ID NO: 30, optionally with up to 10 (e.g., 1, 2, 3, 4, 5, 6, 7, 8, 9, or 10) substitutions (e.g., conservative substitutions) and SEQ ID NO: 31, optionally with up to 10 (e.g., 1, 2, 3, 4, 5, 6, 7, 8, 9, or 10) substitutions (e.g., conservative substitutions).

Provided herein is an antibody or antigen binding fragment thereof comprising the amino acid sequence of SEQ ID NO: 41, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequence of SEQ ID NO: 42, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequences of SEQ ID NO: 41, optionally with up to 10 (e.g., 1, 2, 3, 4, 5, 6, 7, 8, 9, or 10) substitutions (e.g., conservative substitutions) and SEQ ID NO: 42, optionally with up to 10 (e.g., 1, 2, 3, 4, 5, 6, 7, 8, 9, or 10) substitutions (e.g., conservative substitutions).

Provided herein is an antibody or antigen binding fragment thereof comprising the amino acid sequence of SEQ ID NO: 52, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequence of SEQ ID NO: 53, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequences of SEQ ID NO: 52, optionally with up to 10 (e.g., 1, 2, 3, 4, 5, 6, 7, 8, 9, or 10) substitutions (e.g., conservative substitutions) and SEQ ID NO: 53, optionally with up to 10 (e.g., 1, 2, 3, 4, 5, 6, 7, 8, 9, or 10) substitutions (e.g., conservative substitutions).

Provided herein is an antibody or antigen binding fragment thereof comprising the amino acid sequence of SEQ ID NO: 52, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequence of SEQ ID NO: 55, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequences of SEQ ID NO: 52, optionally with up to 10 (e.g., 1, 2, 3, 4, 5, 6, 7, 8, 9, or 10) substitutions (e.g., conservative substitutions) and SEQ ID NO: 55, optionally with up to 10 (e.g., 1, 2, 3, 4, 5, 6, 7, 8, 9, or 10) substitutions (e.g., conservative substitutions).

Provided herein is an antibody or antigen binding fragment thereof comprising the amino acid sequence of SEQ ID NO: 63, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequence of SEQ ID NO: 64, optionally with up to 10 (e.g., 1, 2, 3, 4, 5, 6, 7, 8, 9, or 10) substitutions (e.g., conservative substitutions). Also provided herein is an antibody or antigen binding fragment thereof comprising the amino acid sequences of SEQ ID NO: 63, optionally with up to 10 (e.g., 1, 2, 3, 4, 5, 6, 7, 8, 9, or 10) substitutions (e.g., conservative substitutions) and SEQ ID NO: 64, optionally with up to 10 (e.g., 1, 2, 3, 4, 5, 6, 7, 8, 9, or 10) substitutions (e.g., conservative substitutions).

In some cases, the antibody or antigen binding fragment thereof comprises a VL and VH region, each separately comprising an amino acid sequence having or having at least 85%, 86%, 87%, 88%, 89%, 90%, 91%, 92%, 93%, 94%, 95%, 96%, 97%, 98%, or 99% identity to the VL and VH sequences of any one of the antibodies described in Table 3, Table 4, Table 5. Table 6, Table 7, or Table 8.

Provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 18. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 19. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequences of SEQ ID NO: 18 and SEQ ID NO: 19.

Provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 30. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 31. Also provided herein is an antibody or antigen binding fragment thereof comprising an amino acid sequence having at least 85%, 86%, 87%, 88%, 89%, 90%, 91%, 92%, 93%, 94%, 95%. 96%, 97%, 98%, or 99% identity to the amino acid sequences of SEQ ID NO: 30 and SEQ ID NO: 31.

Provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 41. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 42. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequences of SEQ ID NO: 41 and SEQ ID NO: 42.

Provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 52. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 53. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequences of SEQ ID NO: 52 and SEQ ID NO: 53.

Provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 52. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 55. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequences of SEQ ID NO: 52 and SEQ ID NO: 55.

Provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 63. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequence of SEQ ID NO: 64. Also provided herein is an antibody or antigen binding fragment thereof comprising an amino acid sequence having at least 85%, 86%, 87%, 88%, 89%, 90%, 91%, 92%, 93%, 94%, 95%, 96%, 97%, 98%, or 99% identity to the amino acid sequences of SEQ ID NO: 63 and SEQ ID NO: 64.

In some cases, the antigen binding fragment is selected from the group consisting of a Fab, a Fab′, a F(ab′)2, a Fv, and a scFv.

In some cases, the antigen binding fragment is a scFv. In some cases, the scFv comprises the sequence of any one of the scFvs described in Table 9. In some cases, the scFv comprises an amino acid sequence having or having at least 85%, 86%, 87%, 88%, 89%, 90%, 91%, 92%, 93%, 94%, 95%, 96%, 97%, 98%, or 99% identity to the scFv sequences of any one of the antibodies described in Table 9.

Provided herein is a scFv comprising the amino acid sequence of SEQ ID NO: 18, optionally with up to 10 (e.g., 1, 2, 3, 4, 5, 6, 7, 8, 9, or 10) substitutions (e.g., conservative substitutions). Also provided herein is a scFv comprising the amino acid sequence of SEQ ID NO: 19, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 18, optionally with up to 10 (e.g., 1, 2, 3, 4, 5, 6, 7, 8, 9, or 10) substitutions (e.g., conservative substitutions) and SEQ ID NO: 19, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 18, optionally with up to 10 (e.g., 1, 2, 3, 4, 5, 6, 7, 8, 9, or 10) substitutions (e.g., conservative substitutions) and SEQ ID NO: 19, optionally with up to 10 (e.g., 1, 2, 3, 4, 5, 6, 7, 8, 9, or 10) substitutions (e.g., conservative substitutions) and a polypeptide linker. In some embodiments, the polypeptide linker is (GGGGS)n wherein n=1-4. In some embodiments, the polypeptide linker is selected from the group consisting of: GGGGS, GGGGSGGGGS (SEQ ID NO: 65), GGGGSGGGGSGGGGS (SEQ ID NO: 66), and GGGGSGGGGSGGGGSGGGGS (SEQ ID NO: 67).

Provided herein is a scFv comprising the amino acid sequence of SEQ ID NO: 30, optionally with up to 10 (e.g., 1, 2, 3, 4, 5, 6, 7, 8, 9, or 10) substitutions (e.g., conservative substitutions). Also provided herein is a scFv comprising the amino acid sequence of SEQ ID NO: 31, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 30, optionally with up to 10 (e.g., 1, 2, 3, 4, 5, 6, 7, 8, 9, or 10) substitutions (e.g., conservative substitutions) and SEQ ID NO: 31, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 30, optionally with up to 10 (e.g., 1, 2, 3, 4, 5, 6, 7, 8, 9, or 10) substitutions (e.g., conservative substitutions) and SEQ ID NO: 31, optionally with up to 10 (e.g., 1, 2, 3, 4, 5, 6, 7, 8, 9, or 10) substitutions (e.g., conservative substitutions) and a polypeptide linker. In some embodiments, the polypeptide linker is (GGGGS)n wherein n=1-4. In some embodiments, the polypeptide linker is selected from the group consisting of GGGGS, GGGGSGGGGS (SEQ ID NO: 65), GGGGSGGGGSGGGGS (SEQ ID NO: 66), and GGGGSGGGGSGGGGSGGGGS (SEQ ID NO: 67).

Provided herein is a scFv comprising the amino acid sequence of SEQ ID NO: 41, optionally with up to 10 (e.g., 1, 2, 3, 4, 5, 6, 7, 8, 9, or 10) substitutions (e.g., conservative substitutions). Also provided herein is a scFv comprising the amino acid sequence of SEQ ID NO: 42, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 41, optionally with up to 10 (e.g., 1, 2, 3, 4, 5, 6, 7, 8, 9, or 10) substitutions (e.g., conservative substitutions) and SEQ ID NO: 42, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 41, optionally with up to 10 (e.g., 1, 2, 3, 4, 5, 6, 7, 8, 9, or 10) substitutions (e.g., conservative substitutions) and SEQ ID NO: 42, optionally with up to 10 (e.g., 1, 2, 3, 4, 5, 6, 7, 8, 9, or 10) substitutions (e.g., conservative substitutions) and a polypeptide linker. In some embodiments, the polypeptide linker is (GGGGS)n wherein n=1-4. In some embodiments, the polypeptide linker is selected from the group consisting of: GGGGS, GGGGSGGGGS (SEQ ID NO: 65), GGGGSGGGGSGGGGS (SEQ ID NO: 66), and GGGGSGGGGSGGGGSGGGGS (SEQ ID NO: 67).

Provided herein is a scFv comprising the amino acid sequence of SEQ ID NO: 52, optionally with up to 10 (e.g., 1, 2, 3, 4, 5, 6, 7, 8, 9, or 10) substitutions (e.g., conservative substitutions). Also provided herein is a scFv comprising the amino acid sequence of SEQ ID NO: 53, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 52, optionally with up to 10 (e.g., 1, 2, 3, 4, 5, 6, 7, 8, 9, or 10) substitutions (e.g., conservative substitutions) and SEQ ID NO: 53, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 52, optionally with up to 10 (e.g., 1, 2, 3, 4, 5, 6, 7, 8, 9, or 10) substitutions (e.g., conservative substitutions) and SEQ ID NO: 53, optionally with up to 10 (e.g., 1, 2, 3, 4, 5, 6, 7, 8, 9, or 10) substitutions (e.g., conservative substitutions) and a polypeptide linker. In some embodiments, the polypeptide linker is (GGGGS)n wherein n=1-4. In some embodiments, the polypeptide linker is selected from the group consisting of: GGGGS, GGGGSGGGGS (SEQ ID NO: 65), GGGGSGGGGSGGGGS (SEQ ID NO: 66), and GGGGSGGGGSGGGGSGGGGS (SEQ ID NO: 67).

Provided herein is a scFv comprising the amino acid sequence of SEQ ID NO: 52, optionally with up to 10 (e.g., 1, 2, 3, 4, 5, 6, 7, 8, 9, or 10) substitutions (e.g., conservative substitutions). Also provided herein is a scFv comprising the amino acid sequence of SEQ ID NO: 55, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 52, optionally with up to 10 (e.g., 1, 2, 3, 4, 5, 6, 7, 8, 9, or 10) substitutions (e.g., conservative substitutions) and SEQ ID NO: 55, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 52, optionally with up to 10 (e.g., 1, 2, 3, 4, 5, 6, 7, 8, 9, or 10) substitutions (e.g., conservative substitutions) and SEQ ID NO: 55, optionally with up to 10 (e.g., 1, 2, 3, 4, 5, 6, 7, 8, 9, or 10) substitutions (e.g., conservative substitutions) and a polypeptide linker. In some embodiments, the polypeptide linker is (GGGGS)n wherein n=1-4. In some embodiments, the polypeptide linker is selected from the group consisting of: GGGGS, GGGGSGGGGS (SEQ ID NO: 65), GGGGSGGGGSGGGGS (SEQ ID NO: 66), and GGGGSGGGGSGGGGSGGGGS (SEQ ID NO: 67).

Provided herein is a scFv comprising the amino acid sequence of SEQ ID NO: 63, optionally with up to 10 (e.g., 1, 2, 3, 4, 5, 6, 7, 8, 9, or 10) substitutions (e.g., conservative substitutions). Also provided herein is a scFv comprising the amino acid sequence of SEQ ID NO: 64, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 63, optionally with up to 10 (e.g., 1, 2, 3, 4, 5, 6, 7, 8, 9, or 10) substitutions (e.g., conservative substitutions) and SEQ ID NO: 64, optionally with up to 10 (e.g., 1, 2, 3, 4, 5, 6, 7, 8, 9, or 10) substitutions (e.g., conservative substitutions). Also provided herein is a scFv comprising the amino acid sequences of SEQ ID NO: 63, optionally with up to 10 (e.g., 1, 2, 3, 4, 5, 6, 7, 8, 9, or 10) substitutions (e.g., conservative substitutions) and SEQ ID NO: 64, optionally with up to 10 (e.g., 1, 2, 3, 4, 5, 6, 7, 8, 9, or 10) substitutions (e.g., conservative substitutions) and a polypeptide linker. In some embodiments, the polypeptide linker is (GGGGS)n wherein n=1-4. In some embodiments, the polypeptide linker is selected from the group consisting of: GGGGS, GGGGSGGGGS (SEQ ID NO: 65), GGGGSGGGGSGGGGS (SEQ ID NO: 66), and GGGGSGGGGSGGGGSGGGGS (SEQ ID NO: 67).

Provided herein is a scFv comprising the sequence of SEQ ID NO: 66, optionally with up to 20 (e.g., 1, 2, 3, 4, 5, 6, 7, 8, 9, 10, 11, 12, 13, 14, 15, 16, 17, 18, 19 or 20) substitutions (e.g., conservative substitutions). Also provided herein is a scFv comprising an amino acid sequence having at least 85%, 86%, 87%, 88%, 89%, 90%, 91%, 92%, 93%, 94%, 95%, 96%, 97%, 98%, or 99% identity to SEQ ID NO: 66.

Provided herein is a scFv comprising the sequence of SEQ ID NO: 68, optionally with up to 20 (e.g., 1, 2, 3, 4, 5, 6, 7, 8, 9, 10, 11, 12, 13, 14, 15, 16, 17, 18, 19 or 20) substitutions (e.g., conservative substitutions). Also provided herein is a scFv comprising an amino acid sequence having at least 85%, 86%, 87%, 88%, 89%, 90%, 91%, 92%, 93%, 94%, 95%, 96%, 97%, 98%, or 99% identity to SEQ ID NO: 68.

Provided herein is a scFv comprising the sequence of SEQ ID NO: 69, optionally with up to 20 (e.g., 1, 2, 3, 4, 5, 6, 7, 8, 9, 10, 11, 12, 13, 14, 15, 16, 17, 18, 19 or 20) substitutions (e.g., conservative substitutions). Also provided herein is a scFv comprising an amino acid sequence having at least 85%, 86%, 87%, 88%, 89%, 90%, 91%, 92%, 93%, 94%, 95%, 96%, 97%, 98%, or 99% identity to SEQ ID NO: 69.

Provided herein is a scFv comprising the sequence of SEQ ID NO: 70, optionally with up to 20 (e.g., 1, 2, 3, 4, 5, 6, 7, 8, 9, 10, 11, 12, 13, 14, 15, 16, 17, 18, 19 or 20) substitutions (e.g., conservative substitutions). Also provided herein is a scFv comprising an amino acid sequence having at least 85%, 86%, 87%, 88%, 89%, 90%, 91%, 92%, 93%, 94%, 95%, 96%, 97%, 98%, or 99% identity to SEQ ID NO: 70.

Provided herein is a scFv comprising the sequence of SEQ ID NO: 71, optionally with up to 20 (e.g., 1, 2, 3, 4, 5, 6, 7, 8, 9, 10, 11, 12, 13, 14, 15, 16, 17, 18, 19 or 20) substitutions (e.g., conservative substitutions). Also provided herein is a scFv comprising an amino acid sequence having at least 85%, 86%, 87%, 88%, 89%, 90%, 91%, 92%, 93%, 94%, 95%, 96%, 97%, 98%, or 99% identity to SEQ ID NO: 71.

Provided herein is a scFv comprising the sequence of SEQ ID NO: 72, optionally with up to 20 (e.g., 1, 2, 3, 4, 5, 6, 7, 8, 9, 10, 11, 12, 13, 14, 15, 16, 17, 18, 19 or 20) substitutions (e.g., conservative substitutions). Also provided herein is a scFv comprising an amino acid sequence having at least 85%, 86%, 87%, 88%, 89%, 90%, 91%, 92%, 93%, 94%, 95%, 96%, 97%, 98%, or 99% identity to SEQ ID NO: 72.

CDRs are defined by a variety of methods/systems by those skilled in the art. These systems and/or definitions have been developed and refined over a number of years and include Kabat, Chothia, IMGT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis (bioinf.org.uk/abysis/sequence_input/key_annotation/key_annotation.cgi)) are available and known to those of skill in the art for analysis of antibody sequences and determination of CDRs.

The specific CDR sequences defined herein are generally based on Kabat definitions. However, it will be understood that reference to a heavy chain CDR or CDRs and/or a light chain CDR or CDRs of a specific antibody will encompass all CDR definitions as known to those of skill in the art, e.g., as shown in the Tables herein. In some embodiments, CDR sequences used will all be identified using the same definitions, i.e., will all be Chothia, all Kabat, all IMGT, and so on.

In some embodiments, a BCMA-binding agent is a variant of an agent described herein. In some embodiments, the variant comprises conservative amino acid substitutions relative to a sequence described herein (e.g., of a CDR sequence, a VH sequence, a VL sequence, a scFv sequence, or a CAR sequence or portion thereof described herein). In some embodiments, a BCMA-binding agent (e.g., an antibody or an antigen binding fragment thereof, e.g., a scFv) comprises (a) a heavy chain CDR1, CDR2, CDR3 from a VH sequence presented herein (e.g., in Table 3, Table 4, Table 5, Table 6, Table 7, or Table 8), or a variant thereof comprising 1, 2, 3, or 4 amino acid substitutions; and/or a light chain CDR1, CDR2, and/or CDR3 from a VL sequence presented herein (e.g., in Table 3, Table 4, Table 5. Table 6, Table 7, or Table 8), or a variant thereof comprising 1, 2, 3, or 4 amino acid substitutions. In some embodiments, the amino acid substitutions are conservative substitutions. In some embodiments, a CDR comprises one conservative amino acid substitution. In some embodiments, a CDR comprises two conservative amino acid substitutions. In some embodiments, a CDR comprises three conservative amino acid substitutions. In some embodiments, a CDR comprises four conservative amino acid substitutions. In some embodiments, the CDR is a heavy chain CDR1. In some embodiments, the CDR is a heavy chain CDR2. In some embodiments, the CDR is a heavy chain CDR3. In some embodiments, the CDR is a light chain CDR1. In some embodiments, the CDR is a light chain CDR2. In some embodiments, the CDR is a light chain CDR3. In some embodiments, the substitutions are made as part of a humanization process. In some embodiments, the substitutions are made as part of a germline humanization process. In some embodiments, the substitutions are made as part of an affinity maturation process. In some embodiments, the substitutions are made as part of an optimization process.

In some embodiments, a BCMA-binding agent (e.g., an antibody or antigen binding fragment thereof, e.g., a scFv) comprises one or more heavy chain or light chain CDRs that have been modified, e.g., to reduce deamidation within the CDR sequence, to remove Asn (N)-glycosylation sites, to remove cysteines, or to remove Asp to reduce isomerization sites, to remove Met/Trp or Lys, e.g., to reduce the likelihood within a CDR sequence of asparagine (N)-glycosylation, cysteinylation, asparagine (Asn) deamidation, aspartate (Asp) isomerization, methionine/tryptophan (Met/Trp) oxidation, and non-enzymatic lysine (Lys) glycation (see. e.g., Haberger et al., MAbs. 2014 Mar. 1; 6(2): 327-339; Lu et al., MAbs. 2019 January; 11(1): 45-57). Deamidation is a chemical reaction in which an amide functional group in the side chain of the amino acids asparagine (N) or glutamine (Q) is removed or converted to another functional group. Generally, asparagine is converted to aspartic acid or isoaspartic acid and glutamine is converted to glutamic acid or polyglutamic acid. In some situations, deamidation may change the structure, function, and/or stability of a polypeptide, potentially resulting in decreased biological activity.

In certain embodiments, a BCMA-binding agent comprises a heavy chain variable region comprising heavy chain CDRs 1, 2, and 3, and a light chain variable region comprising light chain CDRs 1, 2, and 3 of any of the antibodies described in Table 3, Table 4, Table 5, Table 6, Table 7, or Table 8.

In some embodiments, a BCMA-binding agent (e.g., an antibody or antigen binding fragment thereof, e.g., a scFv) comprises a heavy chain variable region or sequence having at least about 80% (e.g., at least 90%, 95%, 97%, 99%, or 100%) sequence identity to a heavy chain variable region sequence presented herein (e.g., in Table 3, Table 4, Table 5, Table 6, Table 7, or Table 8), and/or a light chain variable region having at least 80% (e.g., at least 90%, 95%, 97%, 99%, or 100%) sequence identity to a light chain variable region sequence presented herein (e.g., in Table 3, Table 4, Table 5, Table 6, Table 7, or Table 8).

In some embodiments, a BCMA-binding agent described herein (e.g., an antibody or antigen binding fragment thereof, e.g., a scFv) comprises one or more constant heavy domains (e.g., CH1. CH2 and/or CH3 regions). In some embodiments, the one or more constant regions of the BCMA-binding agent has/have been modified. In some embodiments, the antibodies may comprise modifications to one or more of the three heavy chain constant regions (CH1, CH2 or CH3) and/or to the light chain constant region (CL). In some embodiments, the heavy chain constant region of the modified antibodies comprises at least one human constant region. In some embodiments, the heavy chain constant region of the modified antibodies comprises more than one human constant region. In some embodiments, modifications to the constant region comprise additions, deletions, or substitutions of one or more amino acids in one or more regions. In some embodiments, one or more regions are partially or entirely deleted from the constant regions of the modified antibodies. In some embodiments, the entire CH2 domain has been removed from an antibody (ΔCH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

It is known in the art that the constant region(s) of an antibody mediates several effector functions and these effector functions can vary depending on the isotype of the antibody. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), cell-mediated cytotoxicity (ADCC), antibody-dependent cellular phagocytosis (ADCP), complement-dependent cytotoxicity (CDC), release of inflammatory mediators, placental transfer, and control of immunoglobulin production. In some embodiments, an antibody comprises a variant Fc region. The amino acid sequences of the Fc region of human IgG1, IgG2, IgG3, and IgG4 are known to those of ordinary skill in the art (e.g., a representative human IgG1 Fc region is shown in Lobner et al., Immunol Rev. 2016 March: 270(1): 113-131; see, e.g., Table 23). In some cases, Fc regions with amino acid variations have been identified in native antibodies. In some embodiments, a variant Fc region is engineered with substitutions at specific amino acid positions as compared to a native Fc region. In some embodiments, the Fc region is mutated to alter (reduce) antibody dependent cell-mediated cytotoxicity (ADCC), antibody induced complement dependent cytotoxicity (CDC), and/or antibody dependent cell-mediated phagocytosis (ADCP) (see, e.g., Kang and Jung, Experimental & Molecular Medicine. 2019, 51:1-9; Wang et al., Antibody Therapeutics, January 2021. 4 (1):45-54; Lobner et al., Immunol Rev. 2016 March; 270(1): 113-131). In some embodiments, the Fc region is afucosylated (see, e.g., Yamane-Ohnuki and Satoh, MAbs. 2009 May-June; 1(3): 230-236, which describes methods for production of therapeutic antibodies with controlled levels of fucosylation of Fc region N-glycans).

In some embodiments, the modified antibodies (e.g., modified Fc region) provide for altered effector functions that, in turn, affect the biological profile of the antibody. For example, in some embodiments, the deletion or inactivation (through point mutations or other means) of a constant region enhances Fc receptor binding of the modified antibody as it circulates. In some embodiments, the constant region modifications increase the serum half-life of the antibody. In some embodiments, the constant region modifications reduce the serum half-life of the antibody. In some embodiments, the constant region modifications increase or enhance ADCC and/or complement dependent cytotoxicity (CDC) of the antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites.

Modifications to the constant region of antibodies described herein may be made using well known biochemical or molecular engineering techniques. In some embodiments, antibody variants are prepared by introducing appropriate nucleotide changes into the encoding DNA, and/or by synthesis of the desired antibody or polypeptide. Using these antibody variants it may be possible to enhance the activity or effector function provided by a specific sequence or region while substantially maintaining the structure, binding activity, and other desired characteristics of the modified antibody.

The present disclosure further embraces additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, or solubility. Those skilled in the art will appreciate that amino acid changes may alter post-translational processes of an antibody, such as changing the number or position of glycosylation sites or altering membrane anchoring characteristics.

Variations may be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant are determined by systematically making insertions, deletions, or substitutions in the sequence and testing the resulting variant proteins for activity as compared to the parental antibody.

In some embodiments, variants may include addition of amino acid residues at the amino- and/or carboxyl-terminal end of the antibody or polypeptide. The length of additional amino acids residues may range from one residue to a hundred or more residues. In some embodiments, a variant comprises an N-terminal methionyl residue. In some embodiments, the variant comprises an additional polypeptide/protein, i.e., a fusion protein. In some embodiments, a variant is engineered to be detectable and may comprise a detectable label and/or protein (e.g., an enzyme).

In some embodiments, a cysteine residue not involved in maintaining the proper conformation of an antibody may be substituted or deleted to modulate the antibody's characteristics, for example, to improve oxidative stability and/or prevent aberrant disulfide crosslinking. Conversely, in some embodiments, one or more cysteine residues may be added to create disulfide bond(s) to improve stability.

The variant antibodies or polypeptides described herein may be generated using methods known in the art, including but not limited to, site-directed mutagenesis, alanine scanning mutagenesis, and PCR mutagenesis.

In some embodiments, BCMA-binding agents described herein are chemically modified. In some embodiments, the BCMA-binding agents are anti-BCMA antibodies that have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications may be carried out by known techniques.

The present disclosure encompasses BCMA-binding agents built upon non-immunoglobulin backbones, wherein the agents bind to the same epitope or essentially the same epitope as an anti-BCMA antibody disclosed herein. In some embodiments, a non-immunoglobulin-based binding agent is an agent that competes with an anti-BCMA antibody described herein in a competitive binding assay. In some embodiments, alternative BCMA-binding agents comprise a scaffold protein. Generally, scaffold proteins can be assigned to one of three groups based on the architecture of their backbone (1) scaffolds consisting of α-helices; (2) small scaffolds with few secondary structures or an irregular architecture of α-helices and β-sheets; and (3) scaffolds consisting of predominantly β-sheets. Scaffold proteins include, but are not limited to, anticalins, which are based upon the lipocalin scaffold; adnectins, which are based on the 10^{th} domain of human fibronectin type 3; affibodies, which are based on the B-domain in the Ig-binding region of Staphylococcus aureus protein A; darpins, which are based on ankyrin repeat domain proteins; fynomers, which are based on the SH3 domain of the human Fyn protein kinase; affitins, which are based on Sac7d from Sulfolobus acidocaldarius: affilins, which are based on human γ-B-crystallin or human ubiquitin; avimers, which are based on the A-domains of membrane receptor proteins; knottins (cysteine knot miniproteins), which are based upon a stable 30-amino acid anti-parallel β-strand protein fold; and Kunitz domain inhibitor scaffolds, which are based upon a structure that contains three disulfide bonds and three loops. In some embodiments, a BCMA-binding agent comprises an engineered scaffold protein comprising a heavy chain CDR1, CDR2, and CDR3 and a light chain CDR1, CDR2, and CDR3 shown in any one of Table 3, Table 4, Table 5, Table 6. Table 7, or Table 8.

In general, antigen-antibody interactions are non-covalent and reversible, formed by a combination of hydrogen bonds, hydrophobic interactions, electrostatic and van der Waals forces. When describing the strength of an antigen-antibody complex, the terms affinity and/or avidity are commonly used mentioned. The binding of an antibody to its antigen is a reversible process, and the affinity of the binding is typically reported as an equilibrium dissociation constant (K_{D}). K_{D} is the ratio of an antibody dissociation rate (k_{off}) (how quickly it dissociates from its antigen) to the antibody association rate (kₒₙ) (how quickly it binds to its antigen). In some embodiments, K_{D} values are determined by measuring the kₒₙ and k_{off} rates of a specific antibody/antigen interaction and then using a ratio of these values to calculate the K_{D} value. K_{D} values may be used to evaluate and rank order the strength of individual antibody/antigen interactions. The lower the K_{D} of an antibody, the higher the affinity of the antibody for its target. In some embodiments, affinity is measured using SPR technology in a Biacore system. Avidity gives a measure of the overall strength of an antibody-antigen complex. It is dependent on three major parameters: (i) affinity of the antibody for the target. (ii) valency of both the antibody and antigen, and (iii) structural arrangement of the parts that interact.

In some embodiments, a BCMA-binding agent (e.g., an antibody or antigen binding fragment thereof, e.g., a scFv) binds BCMA (e.g., human BCMA) with a dissociation constant (K_{D}) of about 1 μM or less, about 100 nM or less, about 40 nM or less, about 20 nM or less, about 10 nM or less, about 1 nM or less, about 0.1 nM or less, 50 μM or less. 10 μM or less, or 1 μM or less. In some embodiments, a BCMA-binding agent binds BCMA (e.g., human BCMA) with a K_{D} of about 20 nM or less. In some embodiments, a BCMA-binding agent binds BCMA (e.g., human BCMA) with a K_{D} of about 10 nM or less. In some embodiments, a BCMA-binding agent binds BCMA (e.g., human BCMA) with a K_{D} of about 1 nM or less. In some embodiments, a BCMA-binding agent binds BCMA (e.g., human BCMA) with a K_{D} of about 0.5 nM or less. In some embodiments, a BCMA-binding agent binds BCMA (e.g., human BCMA) with a K_{D} of about 0.1 nM or less. In some embodiments, a BCMA-binding agent binds BCMA (e.g., human BCMA) with a K_{D} of about 50 μM or less. In some embodiments, a BCMA-binding agent binds BCMA (e.g., human BCMA) with a K_{D} of about 25 μM or less. In some embodiments, a BCMA-binding agent binds BCMA (e.g., human BCMA) with a K_{D} of about 10 μM or less. In some embodiments, a BCMA-binding agent binds BCMA (e.g., human BCMA) with a K_{D} of about 1 μM or less. In some embodiments, the dissociation constant of the binding agent (e.g., an antibody or antigen binding fragment thereof, e.g., a scFv) for BCMA is the dissociation constant determined using a BCMA protein immobilized on a Biacore chip and the binding agent flowed over the chip. In some embodiments, the dissociation constant of the binding agent (e.g., an antibody or antigen binding fragment thereof, e.g., a scFv) for BCMA is the dissociation constant determined using the binding agent captured by an anti-human IgG antibody on a Biacore chip and soluble BCMA flowed over the chip.

In some embodiments, a BCMA-binding agent (e.g., an antibody or antigen binding fragment thereof, e.g., a scFv) binds BCMA (e.g., human BCMA) with a half maximal effective concentration (EC50) of about 1 μM or less, about 100 nM or less, about 40 nM or less, about 20 nM or less, about 10 nM or less, about 1 nM or less, or about 0.1 nM or less. In some embodiments, a BCMA-binding agent binds to human BCMA with an EC50 of about 1 μM or less, about 100 nM or less, about 40 nM or less, about 20 nM or less, about 10 nM or less, about 1 nM or less, or about 0.1 nM or less. In some embodiments, a BCMA-binding agent binds BCMA with an EC50 of about 40 nM or less, about 20 nM or less, about 10 nM or less, about 1 nM or less or about 0.1 nM or less.

The BCMA-binding agents (e.g., antibodies or antigen binding fragments thereof, e.g., scFvs) described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof. In some embodiments, a DNA sequence encoding a polypeptide of interest is constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In some embodiments, recombinant expression vectors are used to amplify and express DNA encoding antibodies, or fragments thereof, against human BCMA. For example, recombinant expression vectors can be replicable DNA constructs which have synthetic or cDNA-derived DNA fragments encoding a polypeptide chain of a BCMA-binding agent, such as an anti-BCMA antibody, or antigen-binding fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue. This residue can optionally be subsequently cleaved from the expressed recombinant protein to provide a final product.

The choice of an expression control sequence and an expression vector generally depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

The BCMA-binding agents (e.g., antibodies or antigen binding fragments thereof, e.g., scFvs) of the present disclosure can be expressed from one or more vectors. For example, in some embodiments, a heavy chain polypeptide is expressed by one vector and a light chain polypeptide is expressed by a second vector. In some embodiments, a heavy chain polypeptide and a light chain polypeptide are expressed by one vector.

Suitable host cells for expression of a BCMA-binding agent (e.g., an antibody or antigen binding fragment thereof, e.g., a scFv) or a BCMA protein or fragment thereof to use as an antigen or immunogen include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described herein. Cell-free translation systems may also be employed. Appropriate cloning and expression vectors for use with bacterial, fungal, yeast, and mammalian cellular hosts, as well as methods of protein production, including antibody production are well known in the art.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art. Thus, the present disclosure provides cells comprising the BCMA-binding agents described herein. In some embodiments, the cells produce the BCMA-binding agents described herein. In some embodiments, the cell is a prokaryotic cell (e.g., E. coli). In some embodiments, the cell is a eukaryotic cell. In some embodiments, the cell is a mammalian cell. In some embodiments, the cell is a hybridoma cell. Proteins produced by a host cell can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Affinity chromatography used for purifying immunoglobulins can include Protein A, Protein G, and Protein L chromatography. Isolated proteins can be physically characterized using such techniques as proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF). In some embodiments, supernatants from expression systems which secrete recombinant protein into culture media are first concentrated using a commercially available protein concentration filter, for example, an Amicon® or Millipore Pellicon® R) ultrafiltration unit. Following the concentration step, the concentrate can be applied to a suitable purification matrix. In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, a hydroxyapatite media is employed, including but not limited to, ceramic hydroxyapatite (CHT). In some embodiments, one or more reverse-phase HPLC steps employing hydrophobic RP-HPLC media. e.g., silica gel having pendant methyl or other aliphatic groups, are employed to further purify a recombinant protein. In some embodiments, hydrophobic interaction chromatography (HIC) is used to separate recombinant proteins based on their hydrophobicity. HIC is a useful separation technique for purifying proteins while maintaining biological activity due to the use of conditions and matrices that operate under less denaturing conditions than some other techniques. Some or all of the foregoing purification steps, in various combinations, can be employed to provide a homogeneous recombinant protein.

Anti-BCMA antibodies of the present disclosure may be analyzed for their physical/chemical properties and/or biological activities by various assays known in the art. In some embodiments, an anti-BCMA antibody is tested for its ability to bind BCMA (e.g., human BCMA). Binding assays include, but are not limited to, SPR (e.g., Biacore), ELISA, and FACS. In some embodiments, an anti-BCMA antibody is tested for its ability to induce ADCC, ADCP, and/or CDC, as well as the ability of the antibody to kill BCMA target cells (cell depletion). Assays include, but are not limited to, ADCC cell lysis assays, e.g., that use LDH release and detection of formazan salt. In addition, antibodies may be evaluated for solubility, stability, thermostability, viscosity, expression levels, expression quality, and/or purification efficiency.

In some embodiments, purified anti-BCMA antibodies are characterized by assays including, but not limited to, N-terminal sequencing, amino acid analysis, high pressure liquid chromatography (HPLC), mass spectrometry, ion exchange chromatography, and papain digestion.

### III. POLYNUCLEOTIDES/METHODS OF MAKING BINDING AGENTS

Also provided herein are nucleic acids encoding a polypeptide described herein and vectors, preferably expression vectors, containing the nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include. e.g., replication defective retroviruses, adenoviruses and adeno-associated viruses.

A vector can include a nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein that encode a BCMA-binding agent as described herein.

The recombinant expression vectors of the invention can be designed for expression of BCMA-binding agent proteins in prokaryotic cells. Preferably, the BCMA-binding agents can be expressed in mammalian cells, preferably human cells. See, e.g., Frenzel et al., Front Immunol. 2013; 4: 217. When used in mammalian cells, the expression vector's control functions are often provided by viral regulatory elements. For example, commonly used promoters are derived from polyoma, Adenovirus 2, cytomegalovirus and Simian Virus 40.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell can be used to produce (i.e., express) a BCMA-binding agent protein. Accordingly, the invention further provides methods for producing a BCMA-binding agent protein using the host cells of the invention. In one embodiment, the method includes culturing the host cell of the invention (into which a recombinant expression vector encoding a BCMA-binding agent protein has been introduced) in a suitable medium such that a BCMA-binding agent protein is produced. In another embodiment, the method further includes isolating a BCMA-binding agent protein from the medium or the host cell. In some embodiments, the Fc region is afucosylated (see, e.g., Yamane-Ohnuki and Satoh, MAbs. 2009 May-June; 1(3): 230-236, which describes methods for production of therapeutic antibodies with controlled levels of fucosylation of Fc region N-glycans).

### IV. PHARMACEUTICAL COMPOSITIONS

Also provided herein are pharmaceutical compositions comprising a BCMA-binding agent as described herein as an active ingredient. In various embodiments, the BCMA binding agent is prepared as a pharmaceutical composition, for example as a pharmaceutical composition for use as a medicament.

One skilled in the art can formulate the BCMA-binding agent as a pharmaceutical composition according to known methods.

Pharmaceutical compositions can include a carrier. “Carriers” as used herein can include pharmaceutically acceptable carriers, excipients, or stabilizers that are nontoxic (or relatively non-toxic) to the cell or subject being exposed thereto at the dosages and concentrations employed. Often the physiologically acceptable carrier is an aqueous pH buffered solution. Examples of physiologically acceptable carriers include buffers such as phosphate, citrate, and other organic acids: antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine: monosaccharides, disaccharides, and other carbohydrates including glucose, mannose, or dextrins: chelating agents such as EDTA; sugar alcohols such as mannitol or sorbitol; salt-forming counterions such as sodium; and/or nonionic surfactants such as TWEEN™, polyethylene glycol (PEG), and PLURONICS™.

In various embodiments, the BCMA-binding agent is comprised in an injectable formulation, for example, a subcutaneous, intravenous, intramuscular, intrathecal or intraperitoneal injection formulation. Injectable formulations can be aqueous solutions, for example in physiologically compatible buffers such as Hanks' solution, Ringer's solution, or physiological saline buffer. The injectable formulation can contain formulator agents such as suspending, stabilizing, and/or dispersing agents. Alternatively, the BCMA-binding agent can be in a dried or powder form for constitution with a suitable vehicle, e.g., sterile pyrogen-free water, before use.

The binding agents of the present disclosure can be formulated in any suitable form for delivery to a target cell/tissue. In some embodiments, a BCMA-binding agent can be formulated as a liposome, microparticle, microcapsule, albumin microsphere, microemulsion, nano-particle, nanocapsule, or macroemulsion. In some embodiments, the pharmaceutical formulation includes an agent of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE).

In some embodiments, a BCMA-binding agent is formulated as a sustained-release preparation. Suitable examples of sustained-release preparations include semi-permeable matrices of solid hydrophobic polymers containing an agent, where the matrices are in the form of shaped articles (e.g., films or microcapsules). Sustained-release matrices include but are not limited to polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

### V. CHIMERIC ANTIGEN RECEPTORS

Described herein are chimeric antigen receptors (CARs) comprising any of the antibody or antigen-binding fragments thereof described herein, as well as fusion proteins comprising the CARs described herein.

In some cases, the CAR comprises: an extracellular domain comprising the VL and VH of any one of the antibodies or antigen binding fragments described herein. In some cases, the CAR comprises an scFv described herein.

In some embodiments, the CAR comprises one or more of: a signal sequence, an extracellular domain, a hinge, a transmembrane domain, and one or more intracellular signaling domain sequences. In some embodiments, the CAR further comprises a spacer sequence.

In some embodiments, the CAR comprises (from N- to C-terminal): a signal sequence, an extracellular domain, a hinge, a spacer, a transmembrane domain, a first signaling domain sequence, a second signaling domain sequence, and a third signaling domain sequence.

In some embodiments, the CAR comprises (from N- to C-terminal): a signal sequence, an extracellular domain, a hinge, a transmembrane domain, a first signaling domain sequence, a second signaling domain sequence, and a third signaling domain sequence.

In some cases, the signal sequence can be cleaved from a mature CAR protein. Such cleavage can be mediated by a signal peptidase and can occur either during or after completion of translocation to generate the mature protein. Thus, in some embodiments, the CAR comprises (from N- to C-terminal): an extracellular domain, a hinge, a spacer, a transmembrane domain, a first signaling domain sequence, a second signaling domain sequence, and a third signaling domain sequence.

In some embodiments, the CAR comprises (from N- to C-terminal): an extracellular domain, a hinge, a transmembrane domain, a first signaling domain sequence, a second signaling domain sequence, and a third signaling domain sequence.

In some embodiments the extracellular domain comprises an antibody or antigen-binding portion thereof.

In some cases, the CAR comprises a signal sequence. In some cases, the signal sequence is a CD8α signal sequence. In some cases, the CD8α signal sequence comprises or consists of SEQ ID NO: 74. In some cases, the CD8α signal sequence comprises or consists of an amino acid sequence having at least 85%, 86%, 87%, 88%, 89%, 90%, 91%, 92%, 93%, 94%, 95%, 96%, 97%, 98%, or 99% identity to SEQ ID NO: 74.

In some cases, the CAR comprises a hinge domain. In some cases, the hinge domain is a CD8α hinge domain. In some cases, the CD8α hinge domain comprises or consists of SEQ ID NO: 76. In some cases, the CD8α hinge domain comprises or consists of an amino acid sequence having at least 85%, 86%, 87%, 88%, 89%, 90%, 91%, 92%, 93%, 94%, 95%, 96%, 97%, 98%, or 99% identity to SEQ ID NO: 76.

In some cases, the CAR comprises a transmembrane domain. In some cases, the transmembrane domain is a CD28 transmembrane domain. In some cases, the CD28 transmembrane domain comprises or consists of SEQ ID NO: 77. In some cases, the CD28 transmembrane domain comprises or consists of an amino acid sequence having at least 85%, 86%, 87%, 88%, 89%, 90%, 91%, 92%, 93%, 94%, 95%, 96%, 97%, 98%, or 99% identity to SEQ ID NO: 77.

In some cases, the CAR comprises an intracellular signaling domain. See, e.g., Sievers et al., “CARs: Beyond T Cells and T Cell-Derived Signaling Domains,” Int. J. Mol. Sci. 21(10):3525 (2020).

In some cases, the intracellular signaling domain comprises one or more of: a CD3ζ intracellular signaling sequence, a CD28 intracellular signaling sequence, a 4-1BB intracellular signaling sequence, an OX40 intracellular signaling sequence, an IL-2RP intracellular signaling sequence, a 2B4 intracellular signaling sequence, and a cytokine inducer sequence. In some cases, the intracellular signaling domain comprises one or more of: a CD28 intracellular signaling sequence, an OX40L intracellular signaling domain, and a CD3ζ intracellular signaling sequence.

In some cases, the cytokine inducer sequence is an IL-7, IL-12, IL-15, IL-18, or IL-23 inducer sequence.

In some cases, the CD28 intracellular signaling sequence comprises or consists of SEQ ID NO: 78. In some cases, the CD28 intracellular signaling sequence comprises or consists of an amino acid sequence having or having at least 85%, 86%, 87%, 88%, 89%, 90%, 91%, 92%, 93%, 94%, 95%, 96%, 97%, 98%, or 99% identity to SEQ ID NO: 78.

In some cases, the OX40L intracellular signaling sequence comprises or consists of SEQ ID NO: 81, SEQ ID NO: 80, or SEQ ID NO: 79. In some cases, the OX40L intracellular signaling sequence comprises or consists of an amino acid sequence having or having at least 85%, 86%, 87%, 88%, 89%, 90%, 91%, 92%, 93%, 94%, 95%, 96%, 97%, 98%, or 99% identity to SEQ ID NO: 81, SEQ ID NO: 80, or SEQ ID NO: 79.

In some cases, the CD3ζ intracellular signaling sequence comprises or consists of SEQ ID NO: 82. In some cases, the CD3ζ intracellular signaling sequence comprises or consists of an amino acid sequence having or having at least 85%, 86%, 87%, 88%, 89%, 90%, 91%, 92%, 93%, 94%, 95%, 96%, 97%, 98%, or 99% identity to SEQ ID NO: 82. In some cases, the intracellular signaling domain comprises or consists of SEQ ID NO: 83. In some cases, the intracellular signaling domain comprises or consists of an amino acid sequence having or having at least 85%, 86%, 87%, 88%, 89%, 90%, 91%, 92%, 93%, 94%, 95%, 96%, 97%, 98%, or 99% identity to SEQ ID NO: 83.

Also provided herein are polynucleotides encoding any of the CARs described herein and a cytokine. In some cases, the cytokine is IL-15. In some cases, IL-15 comprises or consists of SEQ ID NO: 86. In some cases, the IL-15 comprises or consists of an amino acid sequence having or having at least 85%, 86%, 87%, 88%, 89%, 90%, 91%, 92%, 93%, 94%, 95%, 96%, 97%, 98%, or 99% identity to SEQ ID NO: 86. In some cases, the sequence encoding IL-15 comprises or consists of a nucleic acid sequence having or having at least 85%, 86%, 87%, 88%, 89%, 90%, 91%, 92%, 93%, 94%, 95%, 96%, 97%, 98%, or 99% identity to SEQ ID NO: 87.

In some cases, the polynucleotide comprises a sequence encoding a self-cleaving peptide. e.g., between the sequence encoding the cytokine (e.g., IL-15) and the sequence encoding the CAR.

In some embodiments, the self-cleaving peptide is a 2A self-cleaving peptide. In some embodiments, the self-cleaving peptide is a T2A, P2A, E2A, or F2A self-cleaving peptide. In some embodiments, the self-cleaving peptide comprises SEQ ID NO: 3. In some embodiments, the self-cleaving peptide comprises or consists of SEQ ID NO: 3, SEQ ID NO: 84, SEQ ID NO: 4, SEQ ID NO: 5, or SEQ ID NO: 6.

In some cases, the self-cleaving peptide is T2A. In some cases, the T2A cleavage site comprises or consists of SEQ ID NO: 84. In some cases, the sequence encoding the T2A site comprises or consists of SEQ ID NO: 85.

TABLE 1
Examples of 2A Self-Cleaving Peptides

| | |
|---|---|
| SEQ ID NO: 3 | GDVEXNPGP |
| 2A cleavage motif | |
| SEQ ID NO: 84 | GSGEGRGSLLTCGDVEENPGP |
| T2A cleavage site | |
| SEQ ID NO: 85 | GGCTCAGGTGAGGGGCGCGGGAGCC |
| T2A cleavage site | TGCTGACTTGTGGGGATGTAGAGGA |
| | AAATCCTGGTCCT |
| SEQ ID NO: 4 | GSGATNFSLLKQAGDVEENPGP |
| P2A cleavage site | |
| SEQ ID NO: 5 | GSGQCTNYALLKLAGDVESNPGP |
| E2A cleavage site | |
| SEQ ID NO: 6 | GSGVKQTLNFDLLKLAGDVESNPGP |
| F2A cleavage site | |

In some cases, the CAR comprises a signal sequence (e.g. as described herein), a scFv described herein, a hinge domain (e.g. as described herein), a transmembrane domain (e.g. as described herein), and an intracellular signaling domain (e.g. as described herein). In some cases, the fusion protein further comprises a T2A site (e.g. as described herein) and a sequence encoding IL-15 (e.g., as described herein).

Also provided herein are cells expressing any one of the CARs described herein. In some cases, the cell is a mammalian cell. In some cases, the cell is a human cell. In some cases, the cell is an immune cell, e.g., an innate immune cell. In some cases, the cell is a lymphocyte.

In some cases, the cell is a T cell. In some cases, the cell is a α/β T cell. In some cases, the cell is a γ/δ T cell. In some cases, the cell is a natural killer (NK) cell. In some cases, the cell is a natural killer T (NKT) cell.

In some cases, the cell is a myeloid cell. In some cases, the cell is a neutrophil. In some cases, the cell is a monocyte. In some cases, the cell is a macrophage. In some cases, the cell is a myeloid dendritic cell.

Also provided herein are polynucleotides encoding any one of the CARs described herein (including, e.g., any one of the CARs described herein and a cytokine), and cells comprising the polynucleotide encoding any one of the CARs described herein (including, e.g., any one of the CARs described herein and a cytokine).

In some cases, the CAR comprises the sequence of any one of the CARs described in Table 12. In some cases, the CAR comprises an amino acid sequence having or having at least 85%, 86%, 87%, 88%, 89%, 90%, 91%, 92%, 93%, 94%, 95%, 96%, 97%, 98%, or 99% identity to any one of the CAR sequences described in Table 12.

Provided herein is a CAR comprising: a VL sequence of an anti-BCMA antibody described herein, a VH sequence of an anti-BCMA antibody described herein, a hinge domain, a transmembrane domain, and an intracellular signaling domain. In some cases, the VL and VH sequence are joined by a linker, e.g., as described herein, to form an scFv. In some cases, the CAR further comprises an extracellular signaling sequence. In some cases, the CAR comprises, from N- to C-terminus: a) a VL sequence of an anti-BCMA antibody described herein and a VH sequence of an anti-BCMA antibody described herein, optionally joined by a linker to form an scFv (e.g., as described herein): b) a hinge domain: c) a transmembrane domain; and d) an intracellular signaling domain. In some cases, the VL is N-terminal of the VH. In some cases, the VH is N-terminal of the VL. In some cases the CAR further comprises a signal peptide. e.g., N-terminal of the VH and VL sequences. Also provided herein is a polynucleotide encoding the CAR. In some cases, the polynucleotide further comprises a sequence encoding a cytokine (e.g., IL-15). In some cases, the polynucleotide further comprises a sequence encoding a cleavable peptide (e.g., a self-cleaving peptide), e.g., between the sequence encoding the CAR and the sequence encoding the cytokine. In some cases, the polynucleotide encoding the cytokine is 3′ of the polynucleotide encoding the CAR.

Provided herein is a CAR comprising the CDRs of antibody 01P08A, e.g., the VL and VH chains of antibody 01P08A (see Table 3). Thus, provided herein is a CAR comprising SEQ ID NO: 68, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), SEQ ID NO: 88, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), or SEQ ID NO: 100, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). Also provided herein is a CAR comprising an amino acid sequence having at least 85%, 86%, 87%, 88%, 89%, 90%, 91%, 92%, 93%, 94%, 95%, 96%, 97%, 98%, or 99% identity to SEQ ID NO: 68, SEQ ID NO: 88, or SEQ ID NO: 100. Also provided herein is a polynucleotide encoding a CAR comprising the CDRs of antibody 01P08A, e.g., the VL and VH chains of antibody 01P08A (see Table 3). In some cases, the polynucleotide comprises or consists of SEQ ID NO: 89 or SEQ ID NO: 107. In some cases, the polynucleotide comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 89 or SEQ ID NO: 107. In some cases, the polynucleotide further comprises a sequence encoding a cytokine, e.g., IL-15, optionally linked to the sequence encoding the CAR by way of a linker, e.g., a cleavable peptide, e.g., a self-cleaving peptide, e.g., a self-cleaving peptide described herein, e.g., T2A. Thus, in some cases, the polynucleotide encoding the CAR further comprises a nucleic acid sequence encoding SEQ ID NO: 86. In some cases, the polynucleotide encoding the CAR further comprises a nucleic acid sequence encoding an amino acid sequence having at least 85%, 86%, 87%, 88%, 89%, 90%, 91%, 92%, 93%, 94%, 95%, 96%, 97%, 98%, or 99% identity to SEQ ID NO: 86. In some cases, the nucleic acid sequence comprises or consists of SEQ ID NO: 87. In some cases, the nucleic acid sequence comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 87. In some cases, the polynucleotide encoding the CAR and the cytokine comprises or consists of SEQ ID NO: 108. In some cases, the polynucleotide encoding the CAR and the cytokine comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 108.

Provided herein is a CAR comprising the CDRs of antibody 13I04A, e.g., the VL and VH chains of antibody 13I04A (see Table 4). Thus, provided herein is a CAR comprising or consisting of SEQ ID NO: 69, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), SEQ ID NO: 90, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), or SEQ ID NO: 102, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). Also provided herein is a CAR comprising or consisting of an amino acid sequence having at least 85%, 86%, 87%, 88%, 89%, 90%, 91%, 92%, 93%, 94%, 95%, 96%, 97%, 98%, or 99% identity to SEQ ID NO: 69, SEQ ID NO: 90, or SEQ ID NO: 102, a CAR comprising the CDRs of antibody 13I04A, e.g., the VL and VH chains of antibody 13I04A (see Table 4). In some cases, the polynucleotide comprises or consists of SEQ ID NO: 91 or SEQ ID NO: 111. In some cases, the polynucleotide comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 91 or SEQ ID NO: 111. In some cases, the polynucleotide further comprises a sequence encoding a cytokine, e.g., IL-15, optionally linked to the sequence encoding the CAR by way of a linker. e.g., a cleavable peptide. e.g., a self-cleaving peptide, e.g., a self-cleaving peptide described herein, e.g., T2A. Thus, in some cases, the polynucleotide encoding the CAR further comprises a nucleic acid sequence encoding SEQ ID NO: 86. In some cases, the polynucleotide encoding the CAR further comprises a nucleic acid sequence encoding an amino acid sequence having at least 85%, 86%, 87%, 88%, 89%, 90%, 91%, 92%, 93%, 94%, 95%, 96%, 97%, 98%, or 99% identity to SEQ ID NO: 86. In some cases, the nucleic acid sequence comprises or consists of SEQ ID NO: 87. In some cases, the nucleic acid sequence comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 87. In some cases, the polynucleotide encoding the CAR and the cytokine comprises or consists of SEQ ID NO: 112. In some cases, the polynucleotide encoding the CAR and the cytokine comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 112.

Provided herein is a CAR comprising the CDRs of antibody 13L15A, e.g., the VL and VH chains of antibody 13L15A (see Table 5). Thus, provided herein is a CAR comprising or consisting of SEQ ID NO: 70, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), SEQ ID NO: 92, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), or SEQ ID NO: 98, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). Also provided herein is a CAR comprising or consisting of an amino acid sequence having at least 85%, 86%, 87%, 88%, 89%, 90%, 91%, 92%, 93%, 94%, 95%, 96%, 97%, 98%, or 99% identity to SEQ ID NO: 70, SEQ ID NO: 92, or SEQ ID NO: 98. Also provided herein is a polynucleotide encoding a CAR comprising the CDRs of antibody 13L15A, e.g., the VL and VH chains of antibody 13L15A (see Table 5). In some cases, the polynucleotide comprises or consists of SEQ ID NO: 93 or SEQ ID NO: 103. In some cases, the polynucleotide comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 93 or SEQ ID NO: 103. In some cases, the polynucleotide further comprises a sequence encoding a cytokine, e.g., IL-15, optionally linked to the sequence encoding the CAR by way of a linker, e.g., a cleavable peptide, e.g., a self-cleaving peptide, e.g., a self-cleaving peptide described herein, e.g., T2A. Thus, in some cases, the polynucleotide encoding the CAR further comprises a nucleic acid sequence encoding SEQ ID NO: 86. In some cases, the polynucleotide encoding the CAR further comprises a nucleic acid sequence encoding an amino acid sequence having at least 85%, 86%, 87%, 88%, 89%, 90%, 91%, 92%, 93%, 94%, 95%, 96%, 97%, 98%, or 99% identity to SEQ ID NO: 86. In some cases, the nucleic acid sequence comprises or consists of SEQ ID NO: 87. In some cases, the nucleic acid sequence comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 87. In some cases, the polynucleotide encoding the CAR and the cytokine comprises or consists of SEQ ID NO: 104. In some cases, the polynucleotide encoding the CAR and the cytokine comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 104.

Provided herein is a CAR comprising the CDRs of antibody 14D14A_C, e.g., the VL and VH chains of antibody 14D14A_C (see Table 6). Also provided herein is a CAR comprising the CDRs of antibody 14D14A_C, e.g., the VL and VH chains of antibody 14D14A_C (see Table 6).

Provided herein is a CAR comprising the CDRs of antibody 14D14A, e.g., the VL and VH chains of antibody 14D14A (see Table 7). Thus, provided herein is a CAR comprising or consisting of SEQ ID NO: 71, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), SEQ ID NO: 94, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), or SEQ ID NO: 99, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). Also provided herein is a CAR comprising or consisting of an amino acid sequence having at least 85%, 86%, 87%, 88%, 89%, 90%, 91%, 92%, 93%, 94%, 95%, 96%, 97%, 98%, or 99% identity to SEQ ID NO: 71, SEQ ID NO: 94, or SEQ ID NO: 99. Also provided herein is a polynucleotide encoding a CAR comprising the CDRs of antibody 14D14A, e.g., the VL and VH chains of antibody 14D14A (see Table 7). In some cases, the polynucleotide comprises or consists of SEQ ID NO: 95 or SEQ ID NO: 105. In some cases, the polynucleotide comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 95 or SEQ ID NO: 105. In some cases, the polynucleotide further comprises a sequence encoding a cytokine. e.g., IL-15, optionally linked to the sequence encoding the CAR by way of a linker, e.g., a cleavable peptide, e.g., a self-cleaving peptide, e.g., a self-cleaving peptide described herein, e.g., T2A. Thus, in some cases, the polynucleotide encoding the CAR further comprises a nucleic acid sequence encoding SEQ ID NO: 86. In some cases, the polynucleotide encoding the CAR further comprises a nucleic acid sequence encoding an amino acid sequence having at least 85%, 86%, 87%, 88%, 89%, 90%, 91%, 92%, 93%, 94%, 95%, 96%, 97%, 98%, or 99% identity to SEQ ID NO: 86. In some cases, the nucleic acid sequence comprises or consists of SEQ ID NO: 87. In some cases, the nucleic acid sequence comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 87. In some cases, the polynucleotide encoding the CAR and the cytokine comprises or consists of SEQ ID NO: 106. In some cases, the polynucleotide encoding the CAR and the cytokine comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 106.

Provided herein is a CAR comprising the CDRs of antibody 16M16A, e.g., the VL and VH chains of antibody 16M16A (see Table 8). Thus, provided herein is a CAR comprising or consisting of SEQ ID NO: 72, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), SEQ ID NO: 96, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions), or SEQ ID NO: 101, optionally with up to 20 (e.g., 1, 2, 3, 4, 5, 6, 7, 8, 9, 10, 11, 12, 13, 14, 15, 16, 17, 18, 19, or 20) substitutions (e.g., conservative substitutions). Also provided herein is a CAR comprising or consisting of an amino acid sequence having at least 85%, 86%, 87%, 88%, 89%, 90%, 91%, 92%, 93%, 94%, 95%, 96%, 97%, 98%, or 99% identity to SEQ ID NO: 72, SEQ ID NO: 96, or SEQ ID NO: 101. Also provided herein is a polynucleotide encoding a CAR comprising the CDRs of antibody 16M16A, e.g., the VL and VH chains of antibody 16M16A (see Table 8). In some cases, the polynucleotide comprises or consists of SEQ ID NO: 97 or SEQ ID NO: 109. In some cases, the polynucleotide comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 97 or SEQ ID NO: 109. In some cases, the polynucleotide further comprises a sequence encoding a cytokine, e.g., IL-15, optionally linked to the sequence encoding the CAR by way of a linker, e.g., a cleavable peptide, e.g., a self-cleaving peptide, e.g., a self-cleaving peptide described herein, e.g., T2A. Thus, in some cases, the polynucleotide encoding the CAR further comprises a nucleic acid sequence encoding SEQ ID NO: 86. In some cases, the polynucleotide encoding the CAR further comprises a nucleic acid sequence encoding an amino acid sequence having at least 85%, 86%, 87%, 88%, 89%, 90%, 91%, 92%, 93%, 94%, 95%, 96%, 97%, 98%, or 99% identity to SEQ ID NO: 86. In some cases, the nucleic acid sequence comprises or consists of SEQ ID NO: 87. In some cases, the nucleic acid sequence comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 87. In some cases, the polynucleotide encoding the CAR and the cytokine comprises or consists of SEQ ID NO: 110. In some cases, the polynucleotide encoding the CAR and the cytokine comprises or consists of a nucleic acid sequence having at least 85%, 86%, 87%, 88%, 89%, 90%, 91%, 92%, 93%, 94%, 95%, 96%, 97%, 98%, or 99% identity to SEQ ID NO: 110.

Also provided herein are polynucleic acid(s) encoding the CARs described herein, vector(s) comprising the polynucleic acid(s), and cells, e.g., as described herein, comprising the vector(s).

In some cases, the cell is a mammalian cell. In some cases, the cell is a human cell. In some cases, the cell is an immune cell, e.g., an innate immune cell. In some cases, the cell is a lymphocyte. In some cases, the cell is a T cell. In some cases, the cell is a α/β T cell. In some cases, the cell is a γ/δ T cell. In some cases, the cell is a natural killer (NK) cell. In some cases, the cell is a natural killer T (NKT) cell. In some cases, the cell is a myeloid cell. In some cases, the cell is a neutrophil. In some cases, the cell is a monocyte. In some cases, the cell is a macrophage. In some cases, the cell is a myeloid dendritic cell.

In some embodiments, the vector is a lentivirus vector. See, e.g., Milone et al., “Clinical Use of Lentiviral Vectors,” Leukeinia 32:1529-41 (2018). In some embodiments, the vector is a retrovirus vector. In some embodiments, the vector is a gamma retroviral vector. In some embodiments, the vector is a non-viral vector, e.g., a piggyback non-viral vector (PB transposon, see, e.g., Wu et al., “piggyback is a Flexible and Highly Active Transposon as Compared to Sleeping Beauty, Tol2, and MosI in Mammalian Cells,” PNAS 103(41):15008-13 (2006)), a sleeping beauty non-viral vector (SB transposon, see, e.g., Hudecek et al., “Going Non-Viral: the Sleeping Beauty Transposon System Breaks on Through to the Clinical Side,” Critical Reviews in Biochemistry and Molecular Biology 52(4):355-380 (2017)), or an mRNA vector.

### VI. CAR-NK CELLS AND PHARMACEUTICAL COMPOSITIONS COMPRISING THE SAME

Described herein are natural killer (NK) cells engineered to express a CAR described herein and/or IL-15. e.g., human IL-15 (UniProtKB #P40933; NCBI Gene ID #3600). e.g., soluble human IL-15 or an ortholog thereof, or a variant of any of the foregoing. In some embodiments, the IL-15 is expressed as part of a fusion protein further comprising a cleavage site (e.g., a fusion protein with a CAR described herein). In some embodiments, the IL-15 is expressed as part of a polyprotein comprising a self-cleaving peptide such as a T2A ribosomal skip sequence site (sometimes referred to as a self-cleaving site) (e.g., a CAR polyprotein described herein, e.g., a polyprotein expressed from a polynucleotide comprising a CAR described herein and IL-15). See, e.g., Radcliffe & Mitrophanous, “Multiple Gene Products from a Single Vector: ‘Self-Cleaving’ 2A Peptides,” Gene Therapy 11:1673-4 (2004): see also Liu et al., “Systematic Comparison of 2A Peptides for Cloning Multi-Genes in a Polycistronic Vector,” Scientific Reports 7(1):2193 (2017). In some embodiments, the CAR and the IL-15 are expressed as separate proteins. In some embodiments, the CAR and IL-15 are encoded on separate polynucleotides.

In some embodiments, the IL-15 comprises or consists of SEQ ID NO: 86.

In some embodiments, the self-cleaving peptide is a 2A self-cleaving peptide. In some embodiments, the self-cleaving peptide is a T2A, P2A, E2A, or F2A self-cleaving peptide. In some embodiments, the self-cleaving peptide comprises or consists of SEQ ID NO: 3, SEQ ID NO: 84, SEQ ID NO: 4, SEQ ID NO: 5, or SEQ ID NO: 6.

In some cases, the self-cleaving peptide is T2A. In some cases, the T2A cleavage site comprises or consists of SEQ ID NO: 84. In some cases, the sequence encoding the T2A site comprises or consists of SEQ ID NO: 85.

In some cases, the NK cells stably express the CAR and/or IL-15, e.g., at in some cases, at least 70% (e.g., at least 7%, 80%, 85%, 90%, 95%, or 100%) express the CAR and/or IL-15.

In some cases, the NK cells are expanded and stimulated, e.g., as described in WO2022216813.

In some cases, e.g., after having been ex vivo expanded and stimulated, e.g., as described herein, the expanded and stimulated NK cell populations not only have a number/density (e.g., as described above) that could not occur naturally in the human body, but they also differ in their phenotypic characteristics, (e.g., gene expression and/or surface protein expression) with the starting source material or other naturally occurring populations of NK cells.

In some cases, the starting NK cell source is a sample derived from a single individual, e.g., a single cord blood unit that has not been ex vivo expanded. Therefore, in some cases, the expanded and stimulated NK cells share a common lineage, i.e., they all result from expansion of the starting NK cell source, and, therefore, share a genotype via clonal expansion of a population of cells that are, themselves, from a single organism. Yet, they could not occur naturally at the density achieved with ex vivo expansion and also differ in phenotypic characteristics from the starting NK cell source.

In some cases, the population of expanded and stimulated NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion. 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the expanded and stimulated NK cells comprise at least 80%, e.g., at least 90%, at least 95%, at least 99%, or 100% CD56+CD3-cells.

In some embodiments, the expanded and stimulated NK cells are not genetically engineered.

In some embodiments, the expanded and stimulated NK cells do not comprise a CD16 transgene.

In some embodiments, the expanded and stimulated NK cells do not express an exogenous CD16 protein.

The expanded and stimulated NK cells can be characterized, for example, by surface expression, e.g., of one or more of CD16, CD56, CD3, CD38, CD14, CD19, NKG2D, NKp46, NKp30, DNAM-1, and NKp44.

The surface protein expression levels stated herein, in some cases are achieved without positive selection on the particular surface protein referenced. For example, in some cases, the NK cell source, e.g., a single cord unit, comprises both the KIR B allele of the KIR receptor family and the 158 V/V variant of CD16 and is + enriched and CD3(+) depleted, e.g., by gating on CD56+CD3− expression, but no other surface protein expression selection is carried out during expansion and stimulation.

In some embodiments, the expanded and stimulated NK cells, e.g., from expansion and stimulation of a single cord blood unit. e.g., as described above, comprise at least 60%, e.g., at least 70%, at least 80%, at least 90% at least 95%, at least 99%, or 100% NKG2D+ cells.

In some embodiments, the expanded and stimulated NK cells, e.g., from expansion and stimulation of a single cord blood unit. e.g., as described above, comprise at least 60%, e.g., at least 70%, at least 80%, at least 90% at least 95%, at least 99%, or 100% NKp46+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp30+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% DNAM-1+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp44+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% CD94+(KLRD1) cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD3+ cells.

In some embodiments, the expanded and stimulated NK cells. e.g., from expansion and stimulation of a single cord blood unit, e.g., as described above, comprises less than or equal to 20%, e.g., less than or equal to 10%, less than or equal to 5%, less than or equal to 1% or 0% CD14+ cells.

In some embodiments, the expanded and stimulated NK cells. e.g., from expansion and stimulation of a single cord blood unit, e.g., as described above, comprises less than or equal to 20%, e.g., less than or equal to 10%, less than or equal to 5%, less than or equal to 1% or 0% CD19+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CXCR+ cells.

In some embodiments, the expanded and stimulated NK cells. e.g., from expansion and stimulation of a single cord blood unit, e.g., as described above, comprises less than or equal to 20%, e.g., less than or equal to 10%, less than or equal to 5%, less than or equal to 1% or 0% CD122+(IL2RB) cells.

In some embodiments, the expanded and stimulated NK cells. e.g., from expansion and stimulation of a single cord blood unit, e.g., as described above, comprises 90% or more, e.g., 91%, 92%, 93%, 94%, 95%, 96%, 97%, 98%, 99%, or 100% CD3−CD14−CD19−CD16+CD56− cells.

As described herein, the inventors have demonstrated that, surprisingly, the NK cells expanded and stimulated by the methods described herein express CD16 at high levels throughout the expansion and stimulation process, resulting in a cell population with high CD16 expression. The high expression of CD16 obviates the need for engineering the expanded cells to express CD16, which is important for initiating ADCC, and, therefore, a surprising and unexpected benefit of the expansion and stimulation methods described herein. Thus, in some embodiments, the expanded and stimulated NK cells, e.g., from expansion and stimulation of a single cord blood unit, e.g., as described above, comprise 50% or more, e.g., 55%, 60%, 65%, 70%, 75%, 80%, 85%, 90%, or 95% CD16+NK cells.

In some embodiments, the expanded and stimulated NK cells. e.g., from expansion and stimulation of a single cord blood unit, e.g., as described above, comprises both the KIR B allele of the KIR receptor family and the 158 V/V variant of CD16 and comprise 50% or more, e.g., 55%, 60%, 65%, 70%, 75%, 80%, 85%, 90%, or 95% CD16+NK cells.

In some embodiments, the percentage of expanded and stimulated NK cells, e.g., from expansion and stimulation of a single cord blood unit, e.g., as described above, expressing CD16 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKG2D is the same or higher than the percentage of natural killer cells in the seed cells from umbilical cord blood.

In some embodiments, the percentage of expanded and stimulated NK cells. e.g., from expansion and stimulation of a single cord blood unit, e.g., as described above, expressing NKp30 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing DNAM-1 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp44 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp46 is the same or higher than the percentage of natural killer cells in the seed cells from umbilical cord blood.

As described herein, the inventors have also demonstrated that, surprisingly, the NK cells expanded and stimulated by the methods described herein express CD38 at low levels. CD38 is an effective target for certain cancer therapies (e.g., multiple myeloma and acute myeloid leukemia). See, e.g., Jiao et al., “CD38: Targeted Therapy in Multiple Myeloma and Therapeutic Potential for Solid Cancers,” Expert Opinion on Investigational Drugs 29(11):1295-1308 (2020).

Thus, in some embodiments, the expanded and stimulated NK cells, e.g., from expansion and stimulation of a single cord blood unit, e.g., as described above, comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells. e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit. e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells, and 50% or more, e.g., 55%, 60%, 65%, 70%, 75%, 80%, 85%, 90%, or 95% CD16+NK cells.

In some embodiments, the expanded and stimulated NK cells. e.g., from expansion and stimulation of a single cord blood unit, e.g., as described above, comprises both the KIR B allele of the KIR receptor family and the 158 V/V variant of CD16 and comprise: i) 50% or more, e.g., 55%, 60%, 65%, 70%, 75%, 80%, 85%, 90%, or 95% CD16+NK cells; and/or ii) less than or equal to 80% CD38+ cells, e.g., less than or equal to 75%, 70%, 65%, 60%, 55%, 50%, 45%, 40%, 35%, 30%, 25%, or 20% CD38+ cells; and/or iii) at least 60%, e.g., at least 70%, at least 80%, at least 90% at least 95%, at least 99%, or 100% NKG2D+ cells; and/or iv) at least 60%, e.g., at least 70%, at least 80%, at least 90% at least 95%, at least 99%, or 100% NKp46+ cells; and/or v) at least 60%, e.g., at least 70%, at least 80%, at least 90% at least 95%, at least 99%, or 100% NKp30+ cells; and/or vi) at least 60%, e.g., at least 70%, at least 80%, at least 90% at least 95%, at least 99%, or 100% DNAM-1+ cells; and/or vii) at least 60%, e.g., at least 70%, at least 80%, at least 90% at least 95%, at least 99%, or 100% NKp44+ cells; and/or viii) at least 60%, e.g., at least 70%, at least 80%, at least 90% at least 95%, at least 99%, or 100% CD94+(KLRD1) cells; and/or ix) less than or equal to 20%, e.g., less than or equal to 10%, less than or equal to 5%, less than or equal to 1% or 0% CD3+ cells; and/or x) less than or equal to 20%, e.g., less than or equal to 10%, less than or equal to 5%, less than or equal to 1% or 0% CD14+ cells; and/or xi) less than or equal to 20%, e.g., less than or equal to 10%, less than or equal to 5%, less than or equal to 1% or 0% CD19+ cells; and/or xii) less than or equal to 20%, e.g., less than or equal to 10%, less than or equal to 5%, less than or equal to 1% or 0% CXCR+ cells; and/or xiii) less than or equal to 20%, e.g., less than or equal to 10%, less than or equal to 5%, less than or equal to 1% or 0% CD122+ (IL2RB) cells.

In some embodiments, the NK cell is engineered to alter, e.g., reduce, expression of one or more inhibitor receptor genes.

In some embodiments, the inhibitory receptor gene is a HLA-specific inhibitory receptor. In some embodiments, the inhibitory receptor gene is a non-HLA-specific inhibitory receptor.

In some embodiments, the inhibitor receptor gene is selected from the group consisting of KIR, CD94/NKG2A, LILRB1, PD-1, Irp60. Siglec-7. LAIR-1, and combinations thereof.

Also provided herein are pharmaceutical compositions comprising the natural killer cells described herein and dosage units of the pharmaceutical compositions described herein.

In some cases, the dosage unit comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

In some embodiments, the pharmaceutical composition comprises: a) natural killer cell(s) described herein; and b) a cryopreservation composition.

Suitable cryopreservation compositions are described herein.

In some embodiments, the composition is frozen. In some embodiments, the composition has been frozen for at least three months, e.g., at least six months, at least nine months, at least 12 months, at least 15 months, at least 18 months, at least 24 months, or at least 36 months.

In some embodiments, at least 60%, e.g., at least 70%, at least 80%, at least 90% at least 95%, at least 99%, or 100% of the natural killer cells are viable after being thawed.

In some embodiments, the pharmaceutical composition comprises: a) a cryopreservation composition described herein; and b) therapeutic cell(s).

In some embodiments, the therapeutic cell(s) are animal cell(s). In some embodiments, the therapeutic cell(s) are human cell(s).

In some embodiments, the therapeutic cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein, e.g., the CAR-NKs described herein.

In some embodiments, the pharmaceutical composition further comprises: c) a buffer solution. Suitable buffer solutions are described herein, e.g., as for cryopreservation compositions.

In some embodiments, the pharmaceutical composition comprises from or from about 1×10⁷ to or to about 1×10⁹ cells/mL. In some embodiments, the pharmaceutical composition comprises 1×10⁸ cells/mL. In some embodiments, the pharmaceutical composition comprises about 1×10⁸ cells/mL.

In some embodiments, the pharmaceutical composition further comprises an antibody or antigen binding fragment thereof, e.g., an antibody described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens: antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF. Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polvalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Examples of suitable pharmaceutical compositions are described, for example, in WO2017/135631 and WO2022/0133061, each of which is hereby incorporated by reference in its entirety.

### VII. METHODS OF TREATMENT

The disclosure provides methods comprising administering a BCMA-binding agent as described herein, or a pharmaceutical composition comprising a BCMA-binding agent as described herein, to a subject in need thereof. In some embodiments, the subject is a human. In some embodiments, the methods are carried out in vivo (e.g., as opposed to ex vivo). As used herein, “treatment” refers to therapeutic treatment (treating a subject who has a disease); the methods can also be used for prophylactic or preventative measures, wherein the object is to prevent or slow down (lessen) the targeted pathologic condition or disorder, in a subject who does not have the disease. Those in need of treatment can include those already with the disorder, those prone to have the disorder, or those in whom the disorder is to be prevented.

In some cases, the method comprises administering any of the antibodies or antigen-binding fragments thereof, cells expressing the chimeric antigen receptors described herein (e.g., CAR-NK cells), or pharmaceutical compositions comprising any of these (e.g., as described herein) to a patient in need thereof, e.g., a patient suffering from a disorder, e.g., a disorder associated with cancer, e.g., a BCMA+ cancer.

Also provided herein are methods of preventing, reducing and/or inhibiting the recurrence, growth, proliferation, migration and/or metastasis of a cancer cell or population of cancer cells in a subject in need thereof, comprising any of the antibodies or antigen-binding fragments thereof and/or the CAR-NK cells described herein, e.g., to a patient in need thereof.

Also provided herein are methods of enhancing, improving, and/or increasing the response to an anticancer therapy in a subject in need thereof, comprising administering any of the antibodies or antigen-binding fragments thereof described herein, CAR-NK cells described herein, or pharmaceutical compositions described herein to a patient in need thereof.

Also provided herein are methods of depleting B cell(s) (e.g., BCMA+B cell(s)), comprising administering any of the antibodies or antigen-binding fragments thereof, CAR-NK cells, or pharmaceutical compositions described herein to a patient in need thereof. In some cases, the B cell(s) are CD19+/CD20− B cell(s), CD19+/CD20+B cell(s), CD19−/CD20+ B cell(s), or a combination thereof.

In some cases, the B cell(s) are selected from B cell(s) of anorectum, B cell(s) of appendix, B cell(s) of medullary sinus of lymph node, lymph node mantle zone B cell(s), monocytoid B cell(s), CD19-positive B cell(s), and combinations thereof.

In some cases, the CD19-positive B cell(s) are selected from immature B cell(s), mature B cell(s), precursor B cell(s), transitional stage B cell(s), and combinations thereof.

In some cases, the immature B cell(s) are selected from CD38-negative immature B cell(s), fraction E immature B cell(s), and combinations thereof.

In some cases, the mature B cell(s) are selected from B-1 B cell(s), B-2 B cell(s), Be cell(s), Peyer's patch B cell(s), follicular B cell(s), fraction F mature B cell(s), germinal center B cell(s), marginal zone B cell(s) of lymph node, marginal zone B cell(s) of spleen, memory B cell9s), naïve B cell(s), plasmablast(s), regulatory B cell(s), and combinations thereof.

In some cases, the B-1 B cell(s) are selected from B-1a B cell(s), B-1b B cell(s), and combinations thereof. In some cases, the B-2 B cell(s) are selected from Peyer's patch B cell(s), follicular B cell(s), fraction F mature B cell(s), and combinations thereof. In some cases, the follicular B cell(s) are selected from Bm1 B cell(s). Bm2 B cell(s), and combinations thereof. In some cases, the fraction F mature B cell(s) are Bm1 B cell(s). In some cases, the Be cell(s) are selected from Be1 cell(s), Be2 cell(s), and combinations thereof. In some cases, the germinal center B cell(s) are selected from Bm2′ B cell(s), Bm3 B cell(s). Bm3-delta B cell(s), Bm4 B cell(s), centroblast(s), centrocyte(s), tonsil germinal center B cell(s), and combinations thereof.

In some cases, the memory B cell(s) are selected from Bm5 B cell(s), IgD-negative memory B cell(s), IgM memory B cell(s), class switched memory B cell(s), double negative memory B cell(s), unswitched memory B cell(s), and combinations thereof.

In some cases, the IgD-negative memory B cell(s) are selected from Bm5 B cell(s), CD38-negative IgG memory B cell(s), IgD-negative CD38-positive IgG memory B cell(s), IgM memory B cell(s), double negative memory B cell(s), and combinations thereof. In some cases, the double negative memory B cell(s) are selected from IgG-negative double negative memory B cell(s), IgG-positive double negative memory B cell(s), and combinations thereof.

In some cases, the class switched memory B cell(s) are selected from IgA memory B cell(s), IgE memory B cell(s), IgG memory B cell(s), IgG-negative class switched memory B cell9s), and combinations thereof. In some cases, the IgG memory B cell(s) are selected from CD38-negative IgG memory B cell(s), CD38-positive IgG memory B cell(s), and combinations thereof. In some cases, the CD38-positive IgG memory B cell(s) are selected from IgD-negative CD38-positive IgG memory B cell(s), IgD-positive CD38-positive IgG memory B cell(s), and combinations thereof. In some cases, the IgG-negative class switched memory B cell(s) ae selected from CD38-positive IgG-negative class switched memory B cell(s), CD38-positive IgG-negative class switched memory B cell(s), and combinations thereof. In some cases, the CD38-negative IgG-negative class switched memory B cell(s) are selected from CD24-negative CD38-negative IgG-negative class switched memory B cell(s), CD24-positive CD38-negative IgG-negative class switched memory B cell(s), and combinations thereof. In some cases, the CD38-positive IgG-negative class switched memory B cell(s) are selected from B220-low CD38-positive IgG-negative class switched memory B cell(s), B220-positive CD38-positive IgG-negative class switched memory B cell(s), and combinations thereof. In some cases, the B220-positive CD38-positive IgG-negative class switched memory B cell(s) are B220-low CD38-positive IgG-negative class switched memory B cell(s). In some cases, the double negative memory B cell(s) are selected from IgG-negative double negative memory B cell(s). IgG-positive double negative memory B cell(s), and combinations thereof. In some cases, the unswitched memory B cell(s) are selected from CD38-negative unswitched memory B cell(s), CD38-positive unswitched memory B cell(s), and combinations thereof. In some cases, the CD38-negative unswitched memory B cell(s) are selected from B220-low CD38-negative unswitched memory B cell(s), B220-positive CD38-negative unswitched memory B cell(s), and combinations thereof. In some cases, the B220-positive CD38-negative unswitched memory B cell(s) are B220-low CD38-negative unswitched memory B cell(s). In some cases, the CD38-positive unswitched memory B cell(s) are selected from B220-low CD38-positive unswitched memory B cell(s), B220-positive CD38-positive unswitched memory B cell(s), and combinations thereof. In some cases, the B220-positive CD38-positive unstitched memory B cell(s) are B220-low CD38-positive unswitched memory B cell(s). In some cases, the naïve B cell(s) are selected from CD38-negative naïve B cell(s), CD38-positive naïve B cell(s), and combinations thereof. In some cases, the CD38-positive naïve B cell(s) are B220-low CD38-positive naïve B cell(s), B220-positive cd38-positive naïve B cell(s), and combinations thereof. In some cases, the B220-positive CD38-positive naïve B cell(s) are B220-low CD38-positive naïve B cell(s). In some cases, the plasmablast(s) are selected from CD86-positive plasmablast(s), IgA plasmablast(s), IgD plasmablast(s), IgD plasmablast(s), IgE plasmablast(s), IgG plasmablast(s), IgM plasmablast(s), and combinations thereof.

In some cases, the precursor B cell(s) are selected from fraction B/C precursor B cell(s), fraction C′ precursor B cell(s), fraction D precursor B cell(s), late pro-B cell(s), pre-B-I cell(s), pre-B-II cell(s), and combinations thereof. In some cases, the pre-B-II cell(s) are selected from large pre-B-II cell(s), small pre-B-II cell(s), and combinations thereof. In some cases, the large pre-B-II cell(s) are selected from preBCR-negative large pre-B-II cell(s), preBCR-positive large pre-B-II cell(s), and combinations thereof. In some cases, the pre-BCR-positive large pre-B-II cell(s) are CD38-high pre-BCR positive cell(s). In some cases, the small pre-B-II cell(s) are CD22-positive, CD38-low small pre-B cell(s).

In some cases, the transitional stage B cell(s) are selected from T1B cell(s), T2 B cell(s), T3 B cell(s), and combinations thereof.

In some cases, the B cell(s) are autoreactive B cells.

In some cases, the patient is suffering from an immune disorder. In some cases, the autoimmune disorder is selected from Acromegaly, Acquired aplastic anemia, Acquired hemophilia, Primary Agammaglobulinemia, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, Arteriosclerosis, Autoimmune Addison's disease (AAD). Autoimmune autonomic ganglionopathv (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), Autoimmune hyperlipidemia, Autoimmune hypophysitis, Autoimmune inner ear disease (AIED), Autoimmune lymphoproliferative syndrome (ALPS), Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, Autoimmune retinopathy (AIR), Autoimmune sudden sensorineural hearing loss (SNHL), Balo disease, Behget's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA), Cogan's syndrome. Cold agglutinin disease, CREST syndrome limited cutaneous systemic sclerosis, Crohn's disease (CD), Cronkhite-Canada syndrome (CSS), Cryptogenic organizing pneumonia (COP), Dermatitis herpetiformis, Dermatomyositis, Type 1 Diabetes, Discoid lupus, Dressler's syndrome/postmyocardial infarction/postpericardiotomy syndrome. Eczema/Atopic Dermatitis, Endometriosis, Eosinophilic esophagitis, Eosinophilic fasciitis, Erythema nodosum, Essential mixed cryoglobulinemia, Evans syndrome, Fibrosing alveolitis/Idiopathic pulmonary fibrosis (IPF), Giant cell arteritis/temporal arteritis/Horton's disease, Giant Cell Myocarditis, Glomerulonephritis, Goodpasture's syndrome/anti-GBM/anti-TBM disease. Granulomatosis with polyangiitis (GPA)/Wegener's granulomatosis. Graves disease/thyroid eye disease. Guillain-Barre syndrome (GBS), Hashimoto's thyroiditis/chronic lymphocytic thyroiditis/autoimmune thyroiditis, Henoch-Schonlein purpura/IgA vasculitis, Hidradenitis suppurativa, Hurst's disease/acute hemorrhagic leukoencephalitis (AHLE), Hypogammaglobulinemia. IgA nephropathy/Berger's disease, Immune-mediated necrotizing myopathy (IMNM), Immune thrombocytopenia (ITP)/autoimmune thrombocytopenic purpura/autoimmune thrombocytopenia, Inclusion body myositis, IgG4-related sclerosing disease (ISD), Interstitial cystitis, Juvenile idiopathic arthritis/Adult-onset Still's disease, Juvenile polymyositis|Juvenile dermatomyositis|juvenile myositis, Kawasaki disease, Lambert-Eaton myasthenic syndrome (LEMS), Leukocytoclastic vasculitis, Lichen planus, Lichen sclerosus, Ligneous conjunctivitis, Linear IgA disease (LAD)|linear IgA bullous dermatosis (LABD), Lupus nephritis, Lyme disease/chronic Lyme disease/post-treatment Lyme disease syndrome (PTLDS), Lymphocytic colitis/microscopic colitis, Lymphocytic hypophystitis/autoimmune hypophystitis, Meniere's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Neuromyelitis Optica/Devic's disease, Ocular cicatricial pemphigoid, Opsoclonus-myoclonus syndrome (OMS), Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pemicious anemia, POEMS syndrome, Polyarteritis nodosa, Polymyalgia rheumatica, Polymyositis, Postural orthostatic tachycardia syndrome (POTS), Primary biliary cirrhosis (PBC)/primary biliary cholangitis, Primary sclerosing cholangitis (PSC), Psoriasis, Palmoplantar Pustulosis, Psoriatic arthritis, Pulmonary fibrosis, idiopathic (IPF), Pure red cell aplasia (PRCA), Pyoderma gangrenosum, Rasmussen's encephalitis, Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease, Rheumatic fever, Rheumatoid arthritis, Sarcoidosis, Schmidt syndrome/autoimmune polyendocrine syndrome type II, Scleritis, Scleroderma, Sclerosing Mesenteritis/Mesenteric Panniculitis, Serpiginous choroidopathy, Sjögren's syndrome, Stiff person syndrome (SPS), Small fiber sensory neuropathy, Systemic lupus erythematosus (SLE), Subacute bacterial endocarditis (SBE). Subacute cutaneous lupus, Susac syndrome, Sydenham's chorea, Sympathetic ophthalmia, Takayasu's arteritis (vasculitis), Testicular autoimmunity (vasculitis, orchitis), Tolosa-Hunt syndrome, Transverse myelitis™, Tubulointerstitial nephritis uveitis syndrome (TINU), Ulcerative colitis (UC), Undifferentiated connective tissue disease (UCTD), Uveitis anterior/intermediate/posterior, Vasculitis. VEXAS Syndrome, Vitiligo, Vogt-Koyanagi-Harada syndrome (VKH), and combinations thereof.

In some cases, the patient is suffering from a BCMA+ cancer. In some cases, the BCMA+ cancer is a hematological cancer. In some cases, the hematological cancer is multiple myeloma (MM), chronic lymphocytic leukemia, acute B-lymphoblastic leukemia, non-Hodgkin lymphoma (NHL), or Hodgkin lymphoma.

In some cases, the cancer is relapsed or refractory. In some cases, the cancer is relapsed or refractory multiple myeloma.

In some cases, the patient has an elevated serum BCMA concentration, relative to a control. In some cases, the control is a serum BCMA concentration of patient(s) without a BCMA expressing cancer (e.g., MM). In some cases, the control is a previous serum BCMA concentration from the patient. In some cases, the previous serum BCMA concentration is a pre-treatment serum BCMA concentration.

In some cases, the antibodies or antigen-binding fragments thereof, CARs, cells (e.g., CAR-NK cells) described herein, or pharmaceutical compositions described herein are administered once every week, once every two weeks, once every three weeks, or once every four weeks.

In some cases, the antibodies or antigen-binding fragments thereof, CARs, cells (e.g., CAR-NK cells) described herein, or pharmaceutical compositions described herein are administered intravenously.

In some cases, the antibodies or antigen-binding fragments thereof, CARs, cells (e.g., CAR-NK cells) described herein, or pharmaceutical compositions described herein are administered as a monotherapy. In some cases, the antibodies or antigen-binding fragments thereof, cells (e.g., CAR-NK cells) described herein, or pharmaceutical compositions described herein are administered in combination with another therapy.

In some cases, the antibodies or antigen-binding fragments thereof, CARs, cells (e.g., CAR-NK cells) described herein, or pharmaceutical compositions described herein are administered in combination with an anti-CD38 antibody. In some cases, the anti-CD38 antibody is daratumumab or isatuximab. In some cases, the antibodies or antigen-binding fragments thereof, CAR-NK cells described herein, or pharmaceutical compositions described herein are administered in combination with an anti-CD38 antibody and hyaluronidase.

In some cases, the anti-CD38 antibody (e.g., daratumumab) is administered once a week for the first 8 weeks of a treatment period. In some cases, the anti-CD38 antibody is administered once every two weeks from weeks 9 through 24. In some cases, the anti-CD38 antibody is administered once every four weeks from week 25 until treatment period stops. In some cases, the anti-CD38 antibody (e.g., daratumumab) is administered by intravenous infusion. In some cases, the anti-CD38 antibody and CAR-NK cells are administered simultaneously during the treatment period. In some cases, the anti-CD38 antibody and CAR-NK cells are administered separately during the treatment period.

In some cases, the anti-CD38 antibody (e.g., isatuximab) is administered once a week for the first 4 weeks of a treatment period. In some cases, the anti-CD38 antibody is administered once every two weeks from week 5 until treatment period stops. In some cases, the anti-CD38 antibody (e.g., isatuximab) is administered by intravenous infusion. In some cases, the anti-CD38 antibody and CAR-NK cells are administered simultaneously during the treatment period. In some cases, the anti-CD38 antibody and CAR-NK cells are administered separately during the treatment period.

In some cases, the patient is administered exogenous cytokine (e.g., IL-2 cyotkine) during treatment. In some cases, the patient is not administered exogenous cytokine (e.g., IL-2) during treatment.

In some cases, the patient is newly diagnosed with a CD38+ cancer, e.g., multiple myeloma. In some cases, the newly diagnosed patient cannot receive an autologous stem cell transplant. In some cases, the newly diagnosed patient that can receive an autologous stem cell transplant. In some cases, the patient has received at least one prior medicine to treat multiple myeloma. In some cases, the patient has received at least two prior medicines to treat multiple myeloma. In some cases, the patient has received at least three prior medicines to treat multiple myeloma.

As used herein, the terms “treatment,” “treat,” and “treating” refer to reversing, alleviating, delaying the onset of, or inhibiting the progress of a disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, among other things. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “inhibition,” as it relates to cancer and/or cancer cell proliferation, refer to the inhibition of the growth, division, maturation, or viability of cancer cells, and/or causing the death of cancer cells, individually or in aggregate with other cancer cells, by cytotoxicity, nutrient depletion, or the induction of apoptosis.

As used herein, “delaying” development of a disease or disorder, or one or more symptoms thereof, means to defer, hinder, slow, retard, stabilize, and/or postpone development of the disease, disorder, or symptom thereof. This delay can be of varying lengths of time, depending on the history of the disease and/or subject being treated. As is evident to one skilled in the art, a sufficient or significant delay can, in effect, encompass prevention, in that the subject does not develop the disease, disorder, or symptom thereof. For example, a method that “delays” development of cancer is a method that reduces the probability of disease development in a given time frame and/or reduces extent of the disease in a given time frame, when compared to not using the method. Such comparisons may be based on clinical studies, using a statistically significant number of subjects.

As used herein, “prevention” or “preventing” refers to a regimen that protects against the onset of the disease or disorder such that the clinical symptoms of the disease do not develop. Thus, “prevention” relates to administration of a therapy (e.g., administration of atherapeutic substance) to a subject before signs of the disease are detectable in the subject and/or before a certain stage of the disease (e.g., administration of a therapeutic substance to a subject with a cancer that has not yet metastasized). The subject may be an individual at risk of developing the disease or disorder, or at risk of disease progression, e.g., cancer metastasis. Such as an individual who has one or more risk factors known to be associated with development or onset of the disease or disorder. For example, an individual may have mutations associated with the development or progression of a cancer. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

An “increased” or “enhanced” amount (e.g., with respect to antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

Typically, the agent (e.g., the antibodies or antigen-binding fragments thereof, cells (e.g., CAR-NK cells) described herein, or pharmaceutical compositions described herein) is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom can vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

As such, in various embodiments, the term “effective amount” or therapeutically effective amount” is a concentration or amount of the agent that results in achieving a particular stated purpose, e.g., reduction in one or more symptoms of a disease described herein. An “effective amount” of the agent can be determined empirically. Furthermore, a “therapeutically effective amount” is a concentration or amount of the agent which is effective for achieving a stated therapeutic effect. This amount can also be determined empirically.

In some embodiments, treatment with the agents of the disclosure can kill at least about 20%, e.g., at least about 30%, 40%, 50%, 60%, 70%, or 80% of the BCMA expressing cells that are implicated in the pathogenesis of the diseases disclosed herein.

The term “subject” refers to any animal (e.g., a mammal), including, but not limited to, humans and non-human veterinary subjects including non-human primates.

As used herein, reference to “about” or “approximately” a value or parameter includes (and describes) embodiments that are directed to that value or parameter. For example, a description referring to “about X” includes description of “X.” “About” as used herein means plus or minus 10 percent.

In various embodiments, the BCMA-binding agent can be administered by providing an mRNA encoding the binding agent to the subject.

### VIII. EXAMPLES

The following examples are included for illustrative purposes only and are not intended to limit the scope of the invention.

### Example 1: Antibody Generation

Antibodies were generated by immunizing AlivaMab mice with human BCMA and sequenced. The antibody sequences are shown in Table 3, Table 4, Table 5, Table 6, Table 7, and Table 8. Functional cell-based equilibrium binding constants (EC₅₀) were determined by FACS using Scatchard analysis of titration curves of protein-A purified anti-BCMA antibodies against either: 1) HEK-293T cells stably engineered to express full-length human BCMA, or 2) the BCMA expressing human cell-line H-929 (Table 2). Antibodies were titrated from 10 nM, with seven 1:5 dilutions. A positive control, mouse anti-human BCMA antibody (Biolegend, cat #357514) was tested in each experiment. Binding readout was measured via secondary detection, using an anti-mouse IgG-A647 nm (dilution 1:10000).

TABLE 2
Binding Constants

| Antibody | HEK293 EC50 (nM) | NCI-H929 EC50 (nM) |
|---|---|---|
| 01P08A | 1.035 | 3.793 |
| 13I04A | 0.037 | 0.189 |
| 13L15A | 0.051 | 0.235 |
| 14D14A | 0.172 | 0.827 |
| 16M16A | 0.040 | 0.188 |

TABLE 3
Antibody #1 (Clone ID 01P08A); EC₅₀ = 3.793 nM

| Sequence Name & | |
|---|---|
| SEQ ID NO: | Sequence |
| LC CDR1 | SGYSNYK |
| (IMGT Numbering) | |
| SEQ ID NO: 7 | |
| LC CDR2 | VGTGGIVG |
| (IMGT Numbering) | |
| SEQ ID NO: 8 | |
| LC CDR3 | GADHGSGNNFVYV |
| (IMGT Numbering) | |
| SEQ ID NO: 9 | |
| HC CDR1 | GGSISSYY |
| (IMGT Numbering) | |
| SEQ ID NO: 10 | |
| HC CDR2 | IYKSGNN |
| (IMGT Numbering) | |
| SEQ ID NO: 11 | |
| HC CDR3 | ADGGNYFDWFDP |
| (IMGT Numbering) | |
| SEQ ID NO: 12 | |
| LC CDR1 | TLSSGYSNYKVD |
| (Kabat Numbering) | |
| SEQ ID NO: 13 | |
| LC CDR2 | VGTGGIVGSKGD |
| (Kabat Numbering) | |
| SEQ ID NO: 14 | |
| LC CDR3 | GADHGSGNNFVYV |
| (Kabat Numbering) | |
| SEQ ID NO: 9 | |
| HC CDR1 | SYYWN |
| (Kabat Numbering) | |
| SEQ ID NO: 15 | |
| HC CDR2 | RIYKSGNNIYNPSLQS |
| (Kabat Numbering) | |
| SEQ ID NO: 16 | |
| HC CDR3 | GGNYFDWEDP |
| (Kabat Numbering | |
| SEQ ID NO: 17 | |
| VL | QPVLTQPPSASASLGASVTLTCTLSSGYSNYKVDWYQQRPGKGP |
| SEQ ID NO: 18 | RFVMRVGTGGIVGSKGDGIPDRFSVLGSGLIRYLTIKNIQEEDE |
| | SDYHCGADHGSGNNFVYVFGTGTKVTVL |
| VH | QVQLQESGPGLVKPSETLSLTCIVSGGSISSYYWNWIRQPAGKG |
| SEQ ID NO: 19 | LEWIGRIYKSGNNIYNPSLQSRVTMSLDTSKNQFSLKLSSVTAA |
| | DTAVYYCADGGNYFDWEDPWGQGTLVIVSS |

TABLE 4
Antibody #2 (Clone ID 13104A); EC₅₀ = 0.189 nM

| Sequence Name & | |
|---|---|
| SEQ ID NO: | Sequence |
| LC CDR1 | QSVLYSSYNKNY |
| (IMGT Numbering) | |
| SEQ ID NO: 20 | |
| LC CDR2 | WAS |
| (IMGT Numbering) | |
| LC CDR3 | QQYYSTMYS |
| (IMGT Numbering) | |
| SEQ ID NO: 21 | |
| HC CDR1 | GFTSSNYW |
| (IMGT Numbering) | |
| SEQ ID NO: 22 | |
| HC CDR2 | IKHDGSEK |
| (IMGT Numbering) | |
| SEQ ID NO: 23 | |
| HC CDR3 | ARMRPWYYDL |
| (IMGT Numbering) | |
| SEQ ID NO: 24 | |
| LC CDR1 | KSSQSVLYSSYNKNYLA |
| (Kabat Numbering) | |
| SEQ ID NO: 25 | |
| LC CDR2 | WASTRES |
| (Kabat Numbering) | |
| SEQ ID NO: 26 | |
| LC CDR3 | QQYYSTMYS |
| (Kabat Numbering) | |
| SEQ ID NO: 21 | |
| HC CDR1 | NYWMS |
| (Kabat Numbering) | |
| SEQ ID NO: 27 | |
| HC CDR2 | NIKHDGSEKYFVDSVKG |
| (Kabat Numbering) | |
| SEQ ID NO: 28 | |
| HC CDR3 | MRPWYYDL |
| (Kabat Numbering) | |
| SEQ ID NO: 29 | |
| VL | DIVMTQSPDSLAVSLGERATINCKSSQSVLYSSYNKNYLAWYQQKP |
| SEQ ID NO: 30 | GQPPKILIYWASTRESGVPDRFTGSGSGTDFTLTISSLQAEDVAVY |
| | YCQQYYSTMYSFGQGTKLEIK |
| VH | EVOLVESGGGLVQPGGSLRLSCAASGFTSSNYWMSWVRQAPGKGLE |
| SEQ ID NO: 31 | WVANIKHDGSEKYFVDSVKGRFTISRDNANNSLYLQMNSLRDEDTA |
| | VYYCARMRPWYYDLWGRGTLVTVSS |

TABLE 5
Antibody #3 (Clone ID 13L15A); EC50 = 0.235 nM

| Sequence Name & | |
|---|---|
| SEQ ID NO: | Sequence |
| LC CDR1 | SGYSNYR |
| (IMGT Numbering) | |
| SEQ ID NO: 32 | |
| LC CDR2 | VGTGGIVG |
| (IMGT Numbering) | |
| SEQ ID NO: 8 | |
| LC CDR3 | GADHGSGSNFVWV |
| (IMGT Numbering) | |
| SEQ ID NO: 33 | |
| HC CDR1 | GYTFISYL |
| (IMGT Numbering) | |
| SEQ ID NO: 34 | |
| HC CDR2 | INPSVGST |
| (IMGT Numbering) | |
| SEQ ID NO: 35 | |
| HC CDR3 | ARSSRNYWDGAFDI |
| (IMGT Numbering) | |
| SEQ ID NO: 36 | |
| LC CDR1 | TLSSGYSNYRVD |
| (Kabat Numbering) | |
| SEQ ID NO: 37 | |
| LC CDR2 | VGTGGIVGSKGD |
| (Kabat Numbering) | |
| SEQ ID NO: 14 | |
| LC CDR3 | GADHGSGSNFVWV |
| (Kabat Numbering) | |
| SEQ ID NO: 33 | |
| HC CDR1 | SYLLH |
| (Kabat Numbering) | |
| SEQ ID NO: 38 | |
| HC CDR2 | IINPSVGSTSYAQKFQG |
| (Kabat Numbering) | |
| SEQ ID NO: 39 | |
| HC CDR3 | SSRNYWDGAFDI |
| (Kabat Numbering) | |
| SEQ ID NO: 40 | |
| VL | QPVLTQPPSASASLGASVTLTCTLSSGYSNYRVDWYQQRPGKGPRE |
| SEQ ID NO: 41 | VMRVGTGGIVGSKGDGIPDRFSVLGSGLNRYLTIKNIQEEDESDYH |
| | CGADHGSGSNFVWVFGGGTKLTVL |
| VH | QAQLVQSGAEVKRPGASVKVSCKASGYTFISYLLHWVRQAPGOGLE |
| SEQ ID NO: 42 | WMVIINPSVGSTSYAQKFQGRVTMTRDTSTSTLYMELSSLRSEDTA |
| | VYYCARSSRNYWDGAFDIWGQGTMVTVSS |

TABLE 6
Antibody #4 (Clone ID 14D14A_C); EC₅₀ = 0.827 nM

| Sequence Name & | |
|---|---|
| SEQ ID NO: | Sequence |
| LC CDR1 | QSLLHSNGYNY |
| (IMGT Numbering) | |
| SEQ ID NO: 43 | |
| LC CDR2 | LGS |
| (IMGT Numbering) | |
| LC CDR3 | LQALQTPLT |
| (IMGT Numbering) | |
| SEQ ID NO: 44 | |
| HC CDR1 | GGSISSYY |
| (IMGT Numbering) | |
| SEQ ID NO: 10 | |
| HC CDR2 | MYTSGST |
| (IMGT Numbering) | |
| SEQ ID NO: 45 | |
| HC CDR3 | ARERGYYYGGGGWIDP |
| (IMGT Numbering) | |
| SEQ ID NO: 46 | |
| LC CDR1 | RSSQSLLHSNGYNYLD |
| (Kabat Numbering) | |
| SEQ ID NO: 47 | |
| LC CDR2 | LGSNRAS |
| (Kabat Numbering) | |
| SEQ ID NO: 48 | |
| LC CDR3 | LQALQTPLT |
| (Kabat Numbering) | |
| SEQ ID NO: 44 | |
| HC CDR1 | SYYWC |
| (Kabat Numbering) | |
| SEQ ID NO: 49 | |
| HC CDR2 | RMYTSGSTNYNPSLKS |
| (Kabat Numbering) | |
| SEQ ID NO: 50 | |
| HC CDR3 | ERGYYYGGGGWIDP |
| (Kabat Numbering) | |
| SEQ ID NO: 51 | |
| VL | DIVMTQSPLSLPVTPGEPASISCRSSQSLLHSNGYNYLDWYLQKPG |
| SEQ ID NO: 52 | QSPQLLIYLGSNRASGVPDRFSGSGSGTDFTLKISRVEAEDVGVYY |
| | CLQALQTPLTFGGGTKVEIK |
| VH | QVQLQESGPGLVKPSETLSLTCTVSGGSISSYYWCWIRQSAGKGLE |
| SEQ ID NO: 53 | WIGRMYTSGSTNYNPSLKSRVTMSVDTSKNQFSLKLTSVTAADTAV |
| | YYCARERGYYYGGGGWIDPWGQGTLVTVSS |

TABLE 7
Antibody #4 (Clone ID 14D14A)

| Sequence Name & | |
|---|---|
| SEQ ID NO: | Sequence |
| LC CDR1 | QSLLHSNGYNY |
| (IMGT Numbering) | |
| SEQ ID NO: 43 | |
| LC CDR2 | LGS |
| (IMGT Numbering) | |
| LC CDR3 | LQALQTPLT |
| (IMGT Numbering) | |
| SEQ ID NO: 44 | |
| HC CDR1 | GGSISSYY |
| (IMGT Numbering) | |
| SEQ ID NO: 10 | |
| HC CDR2 | MYTSGST |
| (IMGT Numbering) | |
| SEQ ID NO: 45 | |
| HC CDR3 | ARERGYYYGGGGWIDP |
| (IMGT Numbering) | |
| SEQ ID NO: 46 | |
| LC CDR1 | RSSQSLLHSNGYNYLD |
| (Kabat Numbering) | |
| SEQ ID NO: 47 | |
| LC CDR2 | LGSNRAS |
| (Kabat Numbering) | |
| SEQ ID NO: 48 | |
| LC CDR3 | LQALQTPLT |
| (Kabat Numbering) | |
| SEQ ID NO: 44 | |
| HC CDR1 | SYYWS |
| (Kabat Numbering) | |
| SEQ ID NO: 54 | |
| HC CDR2 | RMYTSGSTNYNPSLKS |
| (Kabat Numbering) | |
| SEQ ID NO: 50 | |
| HC CDR3 | ERGYYYGGGGWIDP |
| (Kabat Numbering) | |
| SEQ ID NO: 51 | |
| VL | DIVMTQSPLSLPVTPGEPASISCRSSQSLLHSNGYNYLDWYLQKPG |
| SEQ ID NO: 52 | QSPQLLIYLGSNRASGVPDRFSGSGSGTDFTLKISRVEAEDVGVYY |
| | CLQALQTPLTFGGGTKVEIK |
| VH | QVQLQESGPGLVKPSETLSLTCTVSGGSISSYYWSWIRQSAGKGLE |
| SEQ ID NO: 55 | WIGRMYTSGSTNYNPSLKSRVTMSVDTSKNQFSLKLTSVTAADTAV |
| | YYCARERGYYYGGGGWIDPWGQGTLVTVSS |

TABLE 8
Antibody #5 (Clone ID 16M16A); EC50 = 0.188 nM

| Sequence Name & | |
|---|---|
| SEQ ID NO: | Sequence |
| LC CDR1 | SGYSNYK |
| (IMGT Numbering) | |
| SEQ ID NO: 7 | |
| LC CDR2 | VGTGGIVG |
| (IMGT Numbering) | |
| SEQ ID NO: 8 | |
| LC CDR3 | GADHGSGSNFVYV |
| (IMGT Numbering) | |
| SEQ ID NO: 56 | |
| HC CDR1 | GSTFTSFF |
| (IMGT Numbering) | |
| SEQ ID NO: 57 | |
| HC CDR2 | INPSGGGT |
| (IMGT Numbering) | |
| SEQ ID NO: 58 | |
| HC CDR3 | SRGNYEAFDL |
| (IMGT Numbering) | |
| SEQ ID NO: 59 | |
| LC CDR1 | TLSSGYSNYKVD |
| (Kabat Numbering) | |
| SEQ ID NO: 13 | |
| LC CDR2 | VGTGGIVGSKGD |
| (Kabat Numbering) | |
| SEQ ID NO: 14 | |
| LC CDR3 | GADHGSGSNFVYV |
| (Kabat Numbering) | |
| SEQ ID NO: 56 | |
| HC CDR1 | SFFIH |
| (Kabat Numbering) | |
| SEQ ID NO: 60 | |
| HC CDR2 | LINPSGGGTGYALKFQG |
| (Kabat Numbering) | |
| SEQ ID NO: 61 | |
| HC CDR3 | GNYEAFDL |
| (Kabat Numbering) | |
| SEQ ID NO: 62 | |
| VL | QPVLTQPPSASASLGASVTLTCTLSSGYSNYKVDWYQQRPGKGPRF |
| SEQ ID NO: 63 | VMRVGTGGIVGSKGDGIPDRFSVLGSGLNRYLTIKNIQEEDESDYH |
| | CGADHGSGSNFVYVFGTGTKVTVL |
| VH | QVQLVQSGTEVKKPGASVKVSCKASGSTFTSFFIHWVRQAPGQGLE |
| SEQ ID NO: 64 | WMGLINPSGGGTGYALKFQGRVTMTRDTSTSTVYMELSSLRSEDTA |
| | VYFCSRGNYEAFDLWGHGTMVTVSS |

### Example 2: ScFv Generation

scFvs were created by linking the VL and VH chains with a linker.

TABLE 9
scFv Sequences

| Sequence | |
|---|---|
| Name & SEQ ID NO: | Sequence |
| linker | GGGGS |
| linker | GGGGSGGGGS |
| SEQ ID NO: 65 | |
| linker | GGGGSGGGGSGGGGS |
| SEQ ID NO: 66 | |
| linker | GGGGSGGGGSGGGGSGGGGS |
| SEQ ID NO: 67 | |
| 01P08A scFv | QPVLTQPPSASASLGASVTLTCTLSSGYSNYKVDWYQQRPGKGPRFVM |
| SEQ ID NO: 68 | RVGTGGIVGSKGDGIPDRFSVLGSGLIRYLTIKNIQEEDESDYHCGAD |
| | HGSGNNFVYVFGTGTKVTVLGGGGSGGGGSGGGGSQVQLQESGPVLVK |
| | PSETLSLTCIVSGGSISSYYWNWIRQPAGKGLEWIGRIYKSGNNIYNP |
| | SLQSRVTMSLDTSKNQFSLKLSSVTAADTAVYYCADGGNYFDWFDPWG |
| | QGTLVIVSS |
| 13I04A scFv | DIVMTQSPDSLAVSLGERATINCKSSQSVLYSSYNKNYLAWYQQKPGQ |
| SEQ ID NO: 69 | PPKILIYWASTRESGVPDRFTGSGSGTDFTLTISSLQAEDVAVYYCQQ |
| | YYSTMYSFGQGTKLEIKGGGGSGGGGSGGGGSEVQLVESGGGLVQPGG |
| | SLRLSCAASGFTSSNYWMSWVRQAPGKGLEWVANIKHDGSEKYFVDSV |
| | KGRFTISRDNANNSLYLQMNSLRDEDTAVYYCARMRPWYYDLWGRGTL |
| | VTVSS |
| 13L15A scFv | QPVLTQPPSASASLGASVTLTCTLSSGYSNYRVDWYQQRPGKGPRFVM |
| SEQ ID NO: 70 | RVGTGGIVGSKGDGIPDRFSVLGSGLNRYLTIKNIQEEDESDYHCGAD |
| | HGSGSNFVWVFGGGTKLTVLGGGGSGGGGSGGGGSQAQLVQSGAEVKR |
| | PGASVKVSCKASGYTFISYLLHWVRQAPGQGLEWMVIINPSVGSTSYA |
| | QKFQGRVTMTRDTSTSTLYMELSSLRSEDTAVYYCARSSRNYWDGAFD |
| | IWGQGTMVTVSS |
| 14D14A scFv | DIVMTQSPLSLPVTPGEPASISCRSSQSLLHSNGYNYLDWYLQKPGQS |
| SEQ ID NO: 71 | PQLLIYLGSNRASGVPDRFSGSGSGTDFTLKISRVEAEDVGVYYCLQA |
| | LQTPLTFGGGTKVEIKGGGGSGGGGSGGGGSQVQLQESGPGLVKPSET |
| | LSLTCTVSGGSISSYYWSWIRQSAGKGLEWIGRMYTSGSTNYNPSLKS |
| | RVTMSVDTSKNQFSLKLTSVTAADTAVYYCARERGYYYGGGGWIDPWG |
| | QGTLVTVSS |
| 16M16A scFv | QPVLTQPPSASASLGASVTLTCTLSSGYSNYKVDWYQQRPGKGPRFVM |
| SEQ ID NO: 72 | RVGTGGIVGSKGDGIPDRFSVLGSGLNRYLTIKNIQEEDESDYHCGAD |
| | HGSGSNFVYVFGTGTKVTVLGGGGSGGGGSGGGGSQVQLVQSGTEVKK |
| | PGASVKVSCKASGSTFTSFFIHWVRQAPGQGLEWMGLINPSGGGTGYA |
| | LKFQGRVTMTRDTSTSTVYMELSSLRSEDTAVYFCSRGNYEAFDLWGH |
| | GTMVTVSS |

### Example 3: High Resolution Epitope Mapping

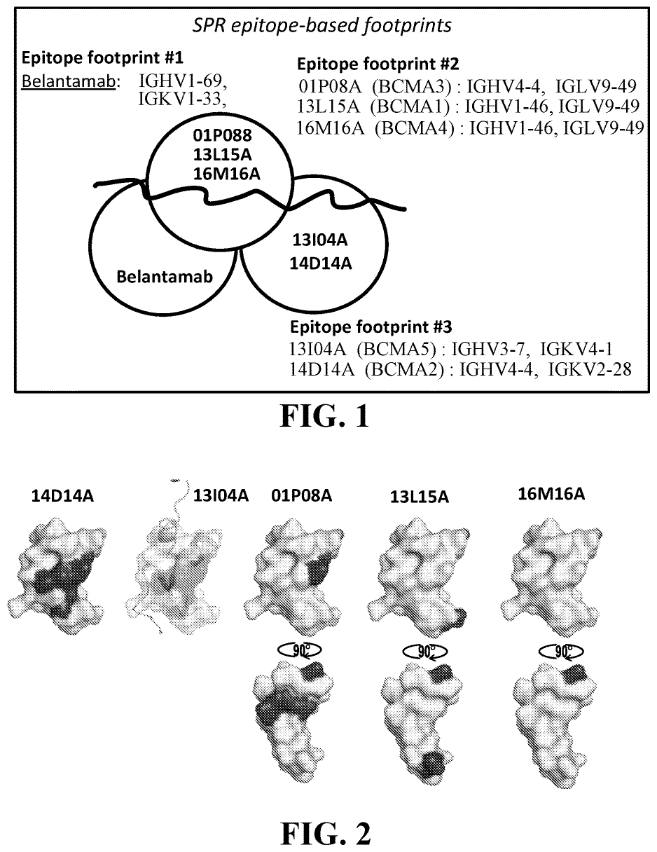

SPR epitope-based footprints reveled that the five scFvs generated in Example 2 (01P08A, 13I04A, 13L15A, 14D14A, and 16M16A) had one of two distinct epitope footprints. Each of those footprints differed from that of belantamab (an anti-BCMA antibody that blocks BCMA-APRIL binding), as shown in FIG. 1.

The five scFvs generated in Example 2 (01P08A, 13I04A, 13L15A, 14D14A, and 16M16A) were evaluated using high-resolution energetic epitope mapping. All 54 residues of the BCMA ectodomain (SEQ ID NO: 69, Table 10) were probed via individual alanine mutations. BCMA was recombinantly expressed on the surface of HEK-293T cells and binding was detected with Alexa Fluor 488 AffiniPure Goat Anti-Mouse IgG (H+L). Critical residues (<20% WT binding and >70% WT binding to a control Ab) were identified.

TABLE 10
BCMA ectodomain sequence

| BCMA | MLQMAGQCSQNEYFDSLLHACIPCQLRCSSNTPPLTC |
|---|---|
| ectodomain | QRYCNASVTNSVKGTNA |
| SEQ ID NO: | |
| 73 | |

The energetic epitope mapping was consistent with the SPR epitope footprints, scFvs 14D14A and 13I04A (footprint 3) cluster onto distinct BCMA surfaces versus 01P08A, 13L15A, and 16M16A (footprint 2). The location of footprint 2 is consistent with the BCMA-APRIL crystal structure.

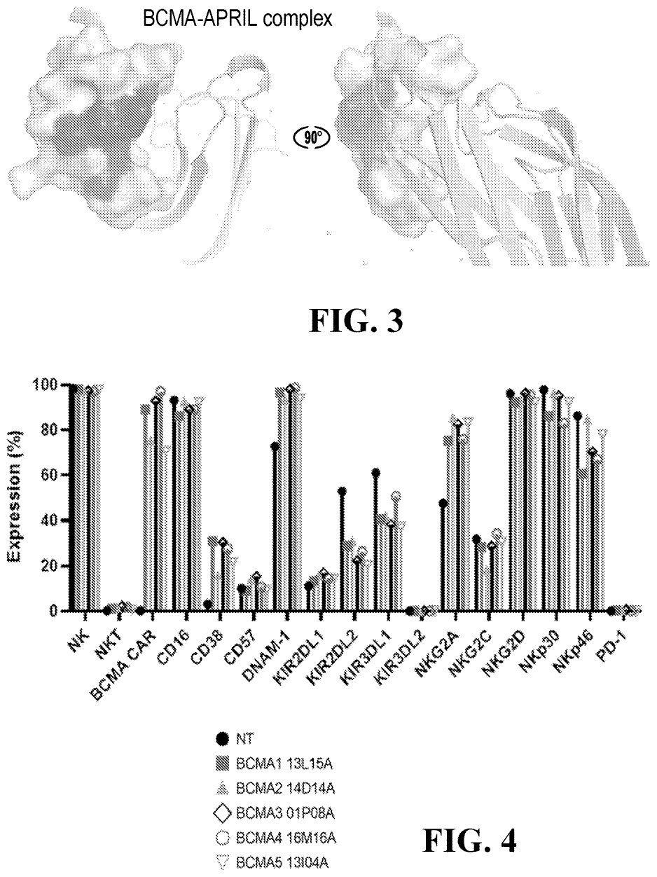

Critical (20% WT binding and >70% WT binding to a control Ab) and important (does not meet critical threshold but causes a significant drop in binding and is near the epitope) epitope residues were identified as shown in Table 11. The crystal structures are shown in FIG. 2. The BCMA-APRIL complex crystal structure, for reference, is shown in FIG. 3.

TABLE 11
Critical and Important Epitope Residues

| Antibody Name | Critical Epitope Residue | Important Epitope Residue |
|---|---|---|
| 13I04A | L2 | |
| 14D14A | E12, I22, P23, L26 | A20 |
| 16M16A | N31 | |
| 01P08A | R27, N31 | L26 |
| 13L15 | L17, N31 | |

### Example 4: ScFv Binding Specificity

The five scFvs generated in Example 2 (01P08A, 13I04A, 13L15A, 14D14A, and 16M16A) were screened for binding against 6105 human plasma membrane proteins, secreted and cell surface-tethered human secreted proteins, plus 400 human heterodimers expressed in human HEK293 cells using a version of the Retrogenix Cell Microarray.

All test scFvs showed a significant specific interaction with the primary target TNFRSF17 (BCMA). Non-specific and low confidence (very weak intensity) interactions were excluded. For 01P08A, 13L15A, 14D14A, and 16M16A, the only specific interaction observed was with the primary target. For 13I04A, one additional significant specific interaction (weak/medium) was observed, with PLA2G2F.

### Example 5: CAR Generation

Chimeric antigen receptors were constructed using the scFvs of Example 2 (01P08A, 13104A, 13L15A, 14D14A, and 16M16A) (VL—linker—VH), with the following scheme (CD8α signal peptide—VL—linker—VH—CD8α hinge—CD28 transmembrane domain—CD28 intracellular signaling sequence—OX40L intracellular signaling sequence—CD3ζ intracellular signaling sequence—T2A site—IL-15):

| | | | | | | | | | | |
|---|---|---|---|---|---|---|---|---|---|---|
| CD8α | VL | Linker | VH | CD8α | CD28 | CD28 | OX40L | CD3ζ | T2A | IL-15 |
| sp | | | | hinge | TM | | | | | |

TABLE 12
CAR Sequences

| Sequence Name & | |
|---|---|
| SEQ ID NO: | Sequence |
| CD8α signal | MALPVTALLLPLALLLHAARP |
| peptide | |
| SEQ ID NO: 74 | |
| CD8α signal | ATGGCCTTACCAGTGACCGCCTTGCTCCTGCCGCTGGCCTTGCTGCTC |
| peptide DNA | CACGCCGCCAGGCCG |
| SEQ ID NO: 75 | |
| CD8α hinge | AKPTTTPAPRPPTPAPTIASQPLSLRPEACRPAAGGAVHTRGLDFACD |
| SEQ ID NO: 76 | |
| CD28 | FWVLVVVGGVLACYSLLVTVAFIIFWV |
| transmembrane | |
| domain | |
| SEQ ID NO: 77 | |
| CD28 intracellular | RSKRSRLLHSDYMNMTPRRPGPTRKHYQPYAPPRDFAAYRS |
| signaling sequence | |
| SEQ ID NO: 78 | |
| OX40L | ERVQPLEENVGNAARPRFERNK |
| intracellular | |
| signaling sequence | |
| SEQ ID NO: 79 | |
| OX40L | LEENVGNAARPRFERNK |
| intracellular | |
| signaling sequence | |
| functional domain | |
| SEQ ID NO: 80 | |
| OX40L | RPRFERNK |
| intracellular | |
| signaling sequence | |
| functional domain | |
| SEQ ID NO: 81 | |
| CD3ζ intracellular | RVKFSRSADAPAYQQGQNQLYNELNLGRREEYDVLDKRRGRDPEMGGK |
| signaling sequence | PRRKNPQEGLYNELQKDKMAEAYSEIGMKGERRRGKGHDGLYQGLSTA |
| SEQ ID NO: 82 | TKDTYDALHMQALPPR |
| Intracellular | ERVQPLEENVGNAARPRFERNK |
| signaling region | RVKFSRSADAPAYQQGQNQLYNELNLGRREEYDVLDKRRGRDPEMGGK |
| SEQ ID NO: 83 | PRRKNPQEGLYNELQKDKMAEAYSEIGMKGERRRGKGHDGLYQGLSTA |
| | TKDTYDALHMQALPPR |
| T2A site | GSGEGRGSLLTCGDVEENPGP |
| SEQ ID NO: 84 | |
| T2A site DNA | GGCTCAGGTGAGGGGCGCGGGAGCCTGCTGACTTGTGGGGATGTAGAG |
| SEQ ID NO: 85 | GAAAATCCTGGTCCT |
| IL-15 | MRISKPHLRSISIQCYLCLLLNSHFLTEAGIHVFILGCFSAGLPKTEA |
| SEQ ID NO: 86 | NWVNVISDLKKIEDLIQSMHIDATLYTESDVHPSCKVTAMKCFLLELQ |
| | VISLESGDASIHDTVENLIILANNSLSSNGNVTESGCKECEELEEKNI |
| | KEFLQSFVHIVQMFINTS |
| IL-15 DNA | ATGAGAATCAGCAAACCACACCTCCGGAGCATATCAATCCAGTGTTAC |
| SEQ ID NO: 87 | TTGTGCCTTCTTTTGAACTCCCATTTCCTCACCGAGGCAGGCATTCAT |
| | GTGTTCATATTGGGGTGCTTTAGTGCTGGGCTTCCGAAAACGGAAGCT |
| | AACTGGGTAAACGTCATCAGTGACCTTAAAAAAATTGAGGATCTTATC |
| | CAATCAATGCACATCGACGCGACTCTCTACACAGAATCTGACGTACAC |
| | CCGTCATGCAAAGTCACGGCAATGAAGTGTTTTCTTCTCGAGCTCCAA |
| | GTAATTTCCCTGGAGTCTGGCGATGCCTCCATCCACGATACGGTTGAA |
| | AATCTGATTATATTGGCCAACAATAGCCTCAGTTCTAACGGTAACGTG |
| | ACTGAAAGTGGCTGCAAAGAGTGCGAAGAGCTCGAAGAAAAGAATATC |
| | AAGGAGTTCCTCCAATCATTTGTTCACATTGTGCAAATGTTTATCAAC |
| | ACCTCTTGA |
| 01P08A CAR | QPVLTQPPSASASLGASVTLTCTLSSGYSNYKVDWYQQRPGKGPRFVM |
| (amino acid) | RVGTGGIVGSKGDGIPDRFSVLGSGLIRYLTIKNIQEEDESDYHCGAD |
| SEQ ID NO: 88 | HGSGNNFVYVFGTGTKVTVLGGGGSGGGGSGGGGSQVQLQESGPGLVK |
| | PSETLSLTCIVSGGSISSYYWNWIRQPAGKGLEWIGRIYKSGNNIYNP |
| | SLQSRVTMSLDTSKNQFSLKLSSVTAADTAVYYCADGGNYFDWFDPWG |
| | QGTLVIVSSAKPTTTPAPRPPTPAPTIASQPLSLRPEACRPAAGGAVH |
| | TRGLDFACDFWVLVVVGGVLACYSLLVTVAFIIFWVRSKRSRLLHSDY |
| | MNMTPRRPGPTRKHYQPYAPPRDFAAYRSERVQPLEENVGNAARPRFE |
| | RNKRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGL |
| | STATKDTYDALHMQALPPR |
| 01P08A CAR | CAGCCAGTCTTGACTCAACCACCAAGCGCGTCCGCAAGTTTGGGTGCT |
| (nucleic acid) | TCTGTTACTTTGACTTGTACACTCAGCTCTGGTTATTCCAATTACAAG |
| SEQ ID NO: 89 | GTGGACTGGTATCAACAGCGGCCTGGAAAGGGGCCGAGATTCGTCATG |
| | CGAGTAGGCACAGGAGGTATCGTCGGAAGTAAAGGTGATGGGATCCCA |
| | GATCGATTCTCCGTACTGGGGTCCGGTCTGATAAGGTACCTCACAATA |
| | AAGAATATCCAGGAGGAAGATGAGTCCGATTACCATTGTGGGGCTGAC |
| | CATGGCAGCGGCAATAATTTCGTTTACGTATTCGGAACCGGGACCAAA |
| | GTCACGGTTCTCGGAGGCGGTGGATCTGGAGGTGGAGGTTCCGGAGGG |
| | GGCGGATCACAGGTGCAGCTTCAGGAGTCTGGCCCTGGATTGGTGAAA |
| | CCTTCCGAAACGCTTAGCTTGACGTGTATCGTCAGTGGTGGCTCAATT |
| | AGCAGCTATTACTGGAATTGGATCCGACAGCCCGCCGGGAAAGGTCTG |
| | GAATGGATAGGGAGAATATACAAAAGCGGGAACAACATATATAACCCC |
| | AGTTTGCAGTCCAGAGTAACTATGTCTCTCGATACTAGTAAGAACCAG |
| | TTCAGCCTTAAACTCAGTTCCGTGACTGCCGCCGACACGGCGGTGTAC |
| | TATTGCGCCGACGGAGGGAATTACTTTGACTGGTTTGACCCGTGGGGT |
| | CAGGGCACTCTTGTGATAGTAAGTTCCGCAAAACCTACCACAACTCCT |
| | GCACCACGCCCCCCTACTCCAGCACCTACCATCGCATCTCAGCCACTG |
| | AGTCTGCGACCAGAGGCCTGCCGGCCCGCCGCCGGCGGGGCCGTCCAT |
| | ACCAGAGGGCTGGACTTTGCCTGCGATTTTTGGGTCCTGGTGGTCGTG |
| | GGAGGGGTGCTGGCATGTTACTCACTGCTGGTCACCGTGGCCTTCATC |
| | ATCTTCTGGGTGCGGAGCAAGAGGTCCCGCCTGCTGCACAGCGACTAT |
| | ATGAACATGACCCCACGGAGACCGGGTCCCACTCGCAAACACTACCAG |
| | CCCTATGCTCCACCCCGGGACTTCGCGGCCTACAGGTCAGAGAGAGTG |
| | CAGCCCCTGGAAGAGAATGTCGGCAACGCTGCCCGCCCAAGATTTGAA |
| | AGGAACAAACGAGTGAAGTTCAGCAGGTCCGCCGACGCTCCTGCATAC |
| | CAGCAGGGACAGAACCAGCTGTATAACGAGCTGAATCTGGGCCGGAGA |
| | GAGGAATACGACGTGCTGGACAAAAGGCGGGGCCGGGACCCCGAAATG |
| | GGAGGGAAGCCACGACGGAAAAACCCCCAGGAGGGCCTGTACAATGAG |
| | CTGCAAAAGGACAAAATGGCCGAGGCTTATTCTGAAATCGGGATGAAG |
| | GGAGAGAGAAGGCGCGGAAAAGGCCACGATGGCCTGTACCAGGGGCTG |
| | AGCACCGCTACAAAGGACACCTATGATGCACTGCACATGCAGGCCCTG |
| | CCCCCTCGG |
| 13I04A CAR | DIVMTQSPDSLAVSLGERATINCKSSQSVLYSSYNKNYLAWYQQKPGQ |
| (amino acid) | PPKILIYWASTRESGVPDRFTGSGSGTDFTLTISSLQAEDVAVYYCQQ |
| SEQ ID NO: 90 | YYSTMYSFGQGTKLEIKGGGGSGGGGSGGGGSEVQLVESGGGLVQPGG |
| | SLRLSCAASGFTSSNYWMSWVRQAPGKGLEWVANIKHDGSEKYFVDSV |
| | KGRFTISRDNANNSLYLQMNSLRDEDTAVYYCARMRPWYYDLWGRGTL |
| | VTVSSAKPTTTPAPRPPTPAPTIASQPLSLRPEACRPAAGGAVHTRGL |
| | DFACDFWVLVVVGGVLACYSLLVTVAFIIFWVRSKRSRLLHSDYMNMT |
| | PRRPGPTRKHYQPYAPPRDFAAYRSERVQPLEENVGNAARPRFERNKR |
| | VKFSRSADAPAYQQGQNQLYNELNLGRREEYDVLDKRRGRDPEMGGKP |
| | RRKNPQEGLYNELQKDKMAEAYSEIGMKGERRRGKGHDGLYQGLSTAT |
| | KDTYDALHMQALPPR |
| 13I04A CAR | GATATAGTCATGACCCAAAGCCCAGATTCCCTTGCGGTATCCCTGGGT |
| (nucleic acid) | GAGAGAGCTACGATCAACTGCAAGAGCTCACAAAGTGTCCTCTACTCC |
| SEQ ID NO: 91 | AGCTATAATAAGAATTACTTGGCGTGGTATCAGCAGAAACCTGGACAG |
| | CCACCAAAAATTCTCATTTACTGGGCTTCCACTCGGGAGAGCGGAGTC |
| | CCGGACCGATTCACAGGTTCCGGTTCTGGAACCGACTTTACCCTGACT |
| | ATTTCCTCCTTGCAGGCGGAAGACGTTGCTGTGTACTATTGCCAACAA |
| | TATTACTCTACAATGTACAGTTTTGGGCAGGGCACCAAACTGGAAATC |
| | AAGGGAGGCGGTGGATCTGGAGGTGGAGGTTCCGGAGGGGGCGGATCA |
| | GAAGTCCAATTGGTTGAATCCGGCGGAGGGCTGGTCCAACCGGGTGGA |
| | TCACTTAGACTTAGCTGTGCAGCGAGCGGCTTTACGTCAAGTAACTAT |
| | TGGATGTCCTGGGTTCGACAAGCACCAGGCAAAGGATTGGAATGGGTA |
| | GCAAACATCAAACATGACGGCAGCGAAAAATATTTCGTTGACTCCGTC |
| | AAGGGAAGGTTTACAATATCACGCGACAATGCTAACAACTCTCTCTAC |
| | CTTCAAATGAATTCTCTCCGAGATGAAGACACAGCCGTCTACTATTGC |
| | GCAAGAATGAGACCTTGGTACTATGACTTGTGGGGCCGAGGAACGCTC |
| | GTGACCGTTTCAAGTGCAAAACCTACCACAACTCCTGCACCACGCCCC |
| | CCTACTCCAGCACCTACCATCGCATCTCAGCCACTGAGTCTGCGACCA |
| | GAGGCCTGCCGGCCCGCCGCCGGCGGGGCCGTCCATACCAGAGGGCTG |
| | GACTTTGCCTGCGATTTTTGGGTCCTGGTGGTCGTGGGAGGGGTGCTG |
| | GCATGTTACTCACTGCTGGTCACCGTGGCCTTCATCATCTTCTGGGTG |
| | CGGAGCAAGAGGTCCCGCCTGCTGCACAGCGACTATATGAACATGACC |
| | CCACGGAGACCGGGTCCCACTCGCAAACACTACCAGCCCTATGCTCCA |
| | CCCCGGGACTTCGCGGCCTACAGGTCAGAGAGAGTGCAGCCCCTGGAA |
| | GAGAATGTCGGCAACGCTGCCCGCCCAAGATTTGAAAGGAACAAACGA |
| | GTGAAGTTCAGCAGGTCCGCCGACGCTCCTGCATACCAGCAGGGACAG |
| | AACCAGCTGTATAACGAGCTGAATCTGGGCCGGAGAGAGGAATACGAC |
| | GTGCTGGACAAAAGGCGGGGCCGGGACCCCGAAATGGGAGGGAAGCCA |
| | CGACGGAAAAACCCCCAGGAGGGCCTGTACAATGAGCTGCAAAAGGAC |
| | AAAATGGCCGAGGCTTATTCTGAAATCGGGATGAAGGGAGAGAGAAGG |
| | CGCGGAAAAGGCCACGATGGCCTGTACCAGGGGCTGAGCACCGCTACA |
| | AAGGACACCTATGATGCACTGCACATGCAGGCCCTGCCCCCTCGG |
| 13L15A CAR | QPVLTQPPSASASLGASVTLTCTLSSGYSNYRVDWYQQRPGKGPRFVM |
| (amino acid) | RVGTGGIVGSKGDGIPDRFSVLGSGLNRYLTIKNIQEEDESDYHCGAD |
| SEQ ID NO: 92 | HGSGSNFVWVFGGGTKLTVLGGGGSGGGGSGGGGSQAQLVQSGAEVKR |
| | PGASVKVSCKASGYTFISYLLHWVRQAPGQGLEWMVIINPSVGSTSYA |
| | QKFQGRVTMTRDTSTSTLYMELSSLRSEDTAVYYCARSSRNYWDGAFD |
| | IWGQGTMVTVSSAKPTTTPAPRPPTPAPTIASQPLSLRPEACRPAAGG |
| | AVHTRGLDFACDFWVLVVVGGVLACYSLLVTVAFIIFWVRSKRSRLLH |
| | SDYMNMTPRRPGPTRKHYQPYAPPRDFAAYRSERVQPLEENVGNAARP |
| | RFERNKRVKFSRSADAPAYQQGQNQLYNELNLGRREEYDVLDKRRGRD |
| | PEMGGKPRRKNPQEGLYNELQKDKMAEAYSEIGMKGERRRGKGHDGLY |
| | QGLSTATKDTYDALHMQALPPR |
| 13L15A CAR | CAACCGGTACTCACACAACCACCGTCAGCAAGTGCCTCCCTTGGCGCT |
| (nucleic acid) | TCTGTTACCCTCACATGCACCTTGTCTTCTGGCTACAGCAACTACCGA |
| SEQ ID NO: 93 | GTAGATTGGTACCAACAACGACCTGGCAAAGGACCTCGGTTCGTCATG |
| | AGGGTCGGGACAGGAGGCATTGTCGGGAGCAAAGGTGACGGAATACCT |
| | GACCGATTCTCCGTCTTGGGGAGTGGGCTTAACCGCTACTTGACCATC |
| | AAGAACATACAAGAAGAAGACGAATCTGACTATCATTGTGGTGCGGAT |
| | CATGGCAGTGGGAGTAACTTCGTTTGGGTTTTTGGTGGAGGTACAAAA |
| | CTGACTGTACTTGGAGGCGGTGGATCTGGAGGTGGAGGTTCCGGAGGG |
| | GGCGGATCACAGGCGCAGCTCGTGCAGAGCGGTGCGGAGGTAAAGCGC |
| | CCAGGGGCTAGCGTGAAAGTTTCATGCAAAGCCTCTGGCTATACGTTT |
| | ATTAGCTACCTGCTGCACTGGGTTAGGCAAGCGCCCGGACAAGGATTG |
| | GAGTGGATGGTGATAATCAATCCCAGTGTGGGTTCCACATCATATGCC |
| | CAGAAGTTCCAAGGGCGAGTTACCATGACTCGAGACACCTCCACATCC |
| | ACCTTGTACATGGAGTTGTCATCCTTGAGATCCGAAGATACGGCTGTC |
| | TACTATTGTGCGCGGTCCAGTCGGAACTATTGGGACGGAGCTTTCGAT |
| | ATATGGGGCCAAGGGACTATGGTTACTGTGTCTTCCGCAAAACCTACC |
| | ACAACTCCTGCACCACGCCCCCCTACTCCAGCACCTACCATCGCATCT |
| | CAGCCACTGAGTCTGCGACCAGAGGCCTGCCGGCCCGCCGCCGGCGGG |
| | GCCGTCCATACCAGAGGGCTGGACTTTGCCTGCGATTTTTGGGTCCTG |
| | GTGGTCGTGGGAGGGGTGCTGGCATGTTACTCACTGCTGGTCACCGTG |
| | GCCTTCATCATCTTCTGGGTGCGGAGCAAGAGGTCCCGCCTGCTGCAC |
| | AGCGACTATATGAACATGACCCCACGGAGACCGGGTCCCACTCGCAAA |
| | CACTACCAGCCCTATGCTCCACCCCGGGACTTCGCGGCCTACAGGTCA |
| | GAGAGAGTGCAGCCCCTGGAAGAGAATGTCGGCAACGCTGCCCGCCCA |
| | AGATTTGAAAGGAACAAACGAGTGAAGTTCAGCAGGTCCGCCGACGCT |
| | CCTGCATACCAGCAGGGACAGAACCAGCTGTATAACGAGCTGAATCTG |
| | GGCCGGAGAGAGGAATACGACGTGCTGGACAAAAGGCGGGGCCGGGAC |
| | CCCGAAATGGGAGGGAAGCCACGACGGAAAAACCCCCAGGAGGGCCTG |
| | TACAATGAGCTGCAAAAGGACAAAATGGCCGAGGCTTATTCTGAAATC |
| | GGGATGAAGGGAGAGAGAAGGCGCGGAAAAGGCCACGATGGCCTGTAC |
| | CAGGGGCTGAGCACCGCTACAAAGGACACCTATGATGCACTGCACATG |
| | CAGGCCCTGCCCCCTCGG |
| 14D14A CAR | DIVMTQSPLSLPVTPGEPASISCRSSQSLLHSNGYNYLDWYLQKPGQS |
| (amino acid) | PQLLIYLGSNRASGVPDRFSGSGSGTDFTLKISRVEAEDVGVYYCLQA |
| SEQ ID NO: 94 | LQTPLTFGGGTKVEIKGGGGSGGGGSGGGGSQVQLQESGPGLVKPSET |
| | LSLTCTVSGGSISSYYWSWIRQSAGKGLEWIGRMYTSGSTNYNPSLKS |
| | RVTMSVDTSKNQFSLKLTSVTAADTAVYYCARERGYYYGGGGWIDPWG |
| | QGTLVTVSSAKPTTTPAPRPPTPAPTIASQPLSLRPEACRPAAGGAVH |
| | TRGLDFACDFWVLVVVGGVLACYSLLVTVAFIIFWVRSKRSRLLHSDY |
| | MNMTPRRPGPTRKHYQPYAPPRDFAAYRSERVQPLEENVGNAARPRFE |
| | RNKRVKESRSADAPAYQQGQNQLYNELNLGRREEYDVLDKRRGRDPEM |
| | GGKPRRKNPQEGLYNELQKDKMAEAYSEIGMKGERRRGKGHDGLYQGL |
| | STATKDTYDALHMQALPPR |
| 14D14A CAR | GATATTGTCATGACCCAGTCCCCTCTCAGTCTCCCGGTAACTCCGGGC |
| (nucleic acid) | GAACCAGCGAGTATTTCTTGTAGGAGTTCCCAGAGCTTGCTGCATAGC |
| SEQ ID NO: 95 | AATGGCTATAATTACTTGGACTGGTACCTGCAAAAACCCGGCCAGTCA |
| | CCCCAACTGCTCATCTATTTGGGATCAAACCGGGCGAGCGGCGTACCC |
| | GACAGGTTCAGTGGCAGCGGTTCCGGAACTGACTTCACTCTGAAAATT |
| | AGTCGCGTAGAAGCCGAGGATGTGGGGGTGTACTACTGCCTCCAGGCC |
| | CTCCAAACTCCCCTTACTTTCGGCGGTGGTACCAAAGTGGAGATAAAA |
| | GGAGGCGGTGGATCTGGAGGTGGAGGTTCCGGAGGGGGCGGATCACAG |
| | GTCCAGCTGCAGGAAAGTGGTCCTGGCCTTGTAAAGCCCTCCGAAACA |
| | CTCTCACTGACATGCACTGTAAGCGGAGGGTCAATTTCCTCTTACTAC |
| | TGGAGCTGGATAAGACAAAGCGCCGGCAAAGGACTCGAGTGGATAGGC |
| | AGGATGTATACCTCTGGGTCAACGAACTACAATCCGTCCCTCAAATCA |
| | CGCGTAACAATGAGCGTGGACACGTCAAAGAACCAATTCTCTTTGAAG |
| | CTTACTAGCGTTACGGCCGCAGACACAGCTGTTTACTATTGCGCGCGC |
| | GAACGCGGTTACTATTATGGTGGAGGGGGGTGGATTGACCCATGGGGT |
| | CAGGGAACCTTGGTCACAGTTTCTTCCGCAAAACCTACCACAACTCCT |
| | GCACCACGCCCCCCTACTCCAGCACCTACCATCGCATCTCAGCCACTG |
| | AGTCTGCGACCAGAGGCCTGCCGGCCCGCCGCCGGCGGGGCCGTCCAT |
| | ACCAGAGGGCTGGACTTTGCCTGCGATTTTTGGGTCCTGGTGGTCGTG |
| | GGAGGGGTGCTGGCATGTTACTCACTGCTGGTCACCGTGGCCTTCATC |
| | ATCTTCTGGGTGCGGAGCAAGAGGTCCCGCCTGCTGCACAGCGACTAT |
| | ATGAACATGACCCCACGGAGACCGGGTCCCACTCGCAAACACTACCAG |
| | CCCTATGCTCCACCCCGGGACTTCGCGGCCTACAGGTCAGAGAGAGTG |
| | CAGCCCCTGGAAGAGAATGTCGGCAACGCTGCCCGCCCAAGATTTGAA |
| | AGGAACAAACGAGTGAAGTTCAGCAGGTCCGCCGACGCTCCTGCATAC |
| | CAGCAGGGACAGAACCAGCTGTATAACGAGCTGAATCTGGGCCGGAGA |
| | GAGGAATACGACGTGCTGGACAAAAGGCGGGGCCGGGACCCCGAAATG |
| | GGAGGGAAGCCACGACGGAAAAACCCCCAGGAGGGCCTGTACAATGAG |
| | CTGCAAAAGGACAAAATGGCCGAGGCTTATTCTGAAATCGGGATGAAG |
| | GGAGAGAGAAGGCGCGGAAAAGGCCACGATGGCCTGTACCAGGGGCTG |
| | AGCACCGCTACAAAGGACACCTATGATGCACTGCACATGCAGGCCCTG |
| | CCCCCTCGG |
| 16M16A CAR | QPVLTQPPSASASLGASVTLTCTLSSGYSNYKVDWYQQRPGKGPRFVM |
| (amino acid) | RVGTGGIVGSKGDGIPDRFSVLGSGLNRYLTIKNIQEEDESDYHCGAD |
| SEQ ID NO: 96 | HGSGSNFVYVFGTGTKVTVLGGGGSGGGGSGGGGSQVQLVQSGTEVKK |
| | PGASVKVSCKASGSTFTSFFIHWVRQAPGQGLEWMGLINPSGGGTGYA |
| | LKFQGRVTMTRDTSTSTVYMELSSLRSEDTAVYFCSRGNYEAFDLWGH |
| | GTMVTVSSAKPTTTPAPRPPTPAPTIASQPLSLRPEACRPAAGGAVHT |
| | RGLDFACDFWVLVVVGGVLACYSLLVTVAFIIFWVRSKRSRLLHSDYM |
| | NMTPRRPGPTRKHYQPYAPPRDFAAYRSERVQPLEENVGNAARPRFER |
| | NKRVKFSRSADAPAYQQGQNQLYNELNLGRREEYDVLDKRRGRDPEMG |
| | GKPRRKNPQEGLYNELQKDKMAEAYSEIGMKGERRRGKGHDGLYQGLS |
| | TATKDTYDALHMQALPPR |
| 16M16A CAR | CAACCTGTGCTGACACAGCCTCCTTCTGCGTCTGCGTCACTGGGAGCT |
| (nucleic acid) | TCCGTAACACTGACCTGTACGCTCTCTTCTGGATATAGCAATTATAAA |
| SEQ ID NO: 97 | GTAGACTGGTACCAACAGAGACCGGGGAAGGGGCCGCGATTCGTTATG |
| | AGAGTTGGTACCGGTGGGATCGTCGGATCAAAAGGGGACGGGATCCCG |
| | GATCGATTTTCCGTTCTTGGGTCCGGTCTCAATAGATATCTGACAATT |
| | AAAAATATACAAGAAGAAGATGAGAGCGACTACCATTGCGGAGCGGAC |
| | CATGGTAGTGGCAGCAACTTTGTCTACGTTTTCGGGACCGGCACAAAG |
| | GTCACCGTACTGGGAGGCGGTGGATCTGGAGGTGGAGGTTCCGGAGGG |
| | GGCGGATCACAGGTCCAGCTCGTTCAGTCTGGAACCGAGGTGAAGAAG |
| | CCAGGTGCATCCGTTAAAGTGTCTTGTAAAGCCTCCGGCTCTACATTC |
| | ACGAGTTTCTTTATACACTGGGTACGCCAGGCGCCTGGCCAGGGTCTT |
| | GAATGGATGGGCCTTATTAATCCATCCGGTGGAGGTACAGGCTATGCA |
| | CTTAAGTTCCAAGGCAGGGTCACCATGACGCGCGACACGTCAACATCA |
| | ACTGTGTATATGGAACTCTCTTCTCTTCGCTCTGAAGACACGGCTGTT |
| | TATTTCTGCAGTCGAGGAAATTACGAGGCATTCGACCTTTGGGGGCAC |
| | GGAACTATGGTGACAGTAAGCTCAGCAAAACCTACCACAACTCCTGCA |
| | CCACGCCCCCCTACTCCAGCACCTACCATCGCATCTCAGCCACTGAGT |
| | CTGCGACCAGAGGCCTGCCGGCCCGCCGCCGGCGGGGCCGTCCATACC |
| | AGAGGGCTGGACTTTGCCTGCGATTTTTGGGTCCTGGTGGTCGTGGGA |
| | GGGGTGCTGGCATGTTACTCACTGCTGGTCACCGTGGCCTTCATCATC |
| | TTCTGGGTGCGGAGCAAGAGGTCCCGCCTGCTGCACAGCGACTATATG |
| | AACATGACCCCACGGAGACCGGGTCCCACTCGCAAACACTACCAGCCC |
| | TATGCTCCACCCCGGGACTTCGCGGCCTACAGGTCAGAGAGAGTGCAG |
| | CCCCTGGAAGAGAATGTCGGCAACGCTGCCCGCCCAAGATTTGAAAGG |
| | AACAAACGAGTGAAGTTCAGCAGGTCCGCCGACGCTCCTGCATACCAG |
| | CAGGGACAGAACCAGCTGTATAACGAGCTGAATCTGGGCCGGAGAGAG |
| | GAATACGACGTGCTGGACAAAAGGCGGGGCCGGGACCCCGAAATGGGA |
| | GGGAAGCCACGACGGAAAAACCCCCAGGAGGGCCTGTACAATGAGCTG |
| | CAAAAGGACAAAATGGCCGAGGCTTATTCTGAAATCGGGATGAAGGGA |
| | GAGAGAAGGCGCGGAAAAGGCCACGATGGCCTGTACCAGGGGCTGAGC |
| | ACCGCTACAAAGGACACCTATGATGCACTGCACATGCAGGCCCTGCCC |
| | CCTCGG |
| 13L15A (CAR with | MALPVTALLLPLALLLHAARPQPVLTQPPSASASLGASVTLTCTLSSG |
| signal peptide) | YSNYRVDWYQQRPGKGPRFVMRVGTGGIVGSKGDGIPDRFSVLGSGLN |
| SEQ ID NO: 98 | RYLTIKNIQEEDESDYHCGADHGSGSNFVWVFGGGTKLTVLGGGGSGG |
| | GGSGGGGSQAQLVQSGAEVKRPGASVKVSCKASGYTFISYLLHWVRQA |
| | PGQGLEWMVIINPSVGSTSYAQKFQGRVTMTRDTSTSTLYMELSSLRS |
| | EDTAVYYCARSSRNYWDGAFDIWGQGTMVTVSSAKPTTTPAPRPPTPA |
| | PTIASQPLSLRPEACRPAAGGAVHTRGLDFACDFWVLVVVGGVLACYS |
| | LLVTVAFIIFWVRSKRSRLLHSDYMNMTPRRPGPTRKHYQPYAPPRDE |
| | AAYRSERVQPLEENVGNAARPRFERNKRVKFSRSADAPAYQQGQNQLY |
| | NELNLGRREEYDVLDKRRGRDPEMGGKPRRKNPQEGLYNELQKDKMAE |
| | AYSEIGMKGERRRGKGHDGLYQGLSTATKDTYDALHMQALPPR |
| 13L15A (T2A) | GSGEGRGSLLTCGDVEENPGP |
| SEQ ID NO: 84 | |
| 13L15A (IL-15) | MRISKPHLRSISIQCYLCLLLNSHFLTEAGIHVFILGCFSAGLPKTEA |
| SEQ ID NO: 86 | NWVNVISDLKKIEDLIQSMHIDATLYTESDVHPSCKVTAMKCFLLELQ |
| | VISLESGDASIHDTVENLIILANNSLSSNGNVTESGCKECEELEEKNI |
| | KEFLQSFVHIVQMFINTS |
| 14D14A (CAR | MALPVTALLLPLALLLHAARPDIVMTQSPLSLPVTPGEPASISCRSSQ |
| with signal peptide) | SLLHSNGYNYLDWYLQKPGQSPQLLIYLGSNRASGVPDRFSGSGSGTD |
| SEQ ID NO: 99 | FTLKISRVEAEDVGVYYCLQALQTPLTFGGGTKVEIKGGGGSGGGGSG |
| | GGGSQVQLQESGPGLVKPSETLSLTCTVSGGSISSYYWSWIRQSAGKG |
| | LEWIGRMYTSGSTNYNPSLKSRVTMSVDTSKNQFSLKLTSVTAADTAV |
| | YYCARERGYYYGGGGWIDPWGQGTLVTVSSAKPTTTPAPRPPTPAPTI |
| | ASQPLSLRPEACRPAAGGAVHTRGLDFACDFWVLVVVGGVLACYSLLV |
| | TVAFIIFWVRSKRSRLLHSDYMNMTPRRPGPTRKHYQPYAPPRDFAAY |
| | RSERVQPLEENVGNAARPRFERNKRVKFSRSADAPAYQQGQNQLYNEL |
| | NLGRREEYDVLDKRRGRDPEMGGKPRRKNPQEGLYNELQKDKMAEAYS |
| | EIGMKGERRRGKGHDGLYQGLSTATKDTYDALHMQALPPR |
| 14D14A (T2A) | GSGEGRGSLLTCGDVEENPGP |
| SEQ ID NO: 84 | |
| 14D14A (IL-15) | MRISKPHLRSISIQCYLCLLLNSHFLTEAGIHVFILGCFSAGLPKTEA |
| SEQ ID NO: 86 | NWVNVISDLKKIEDLIQSMHIDATLYTESDVHPSCKVTAMKCELLELQ |
| | VISLESGDASIHDTVENLIILANNSLSSNGNVTESGCKECEELEEKNI |
| | KEFLQSFVHIVQMFINTS |
| 01P08A (CAR with | MALPVTALLLPLALLLHAARPQPVLTQPPSASASLGASVTLTCTLSSG |
| signal peptide) | YSNYKVDWYQQRPGKGPRFVMRVGTGGIVGSKGDGIPDRFSVLGSGLI |
| SEQ ID NO: 100 | RYLTIKNIQEEDESDYHCGADHGSGNNFVYVFGTGTKVTVLGGGGSGG |
| | GGSGGGGSQVQLQESGPGLVKPSETLSLTCIVSGGSISSYYWNWIRQP |
| | AGKGLEWIGRIYKSGNNIYNPSLQSRVTMSLDTSKNQFSLKLSSVTAA |
| | DTAVYYCADGGNYFDWEDPWGQGTLVIVSSAKPTTTPAPRPPTPAPTI |
| | ASQPLSLRPEACRPAAGGAVHTRGLDFACDFWVLVVVGGVLACYSLLV |
| | TVAFIIFWVRSKRSRLLHSDYMNMTPRRPGPTRKHYQPYAPPRDFAAY |
| | RSERVQPLEENVGNAARPRFERNKRVKFSRSADAPAYQQGQNQLYNEL |
| | NLGRREEYDVLDKRRGRDPEMGGKPRRKNPQEGLYNELQKDKMAEAYS |
| | EIGMKGERRRGKGHDGLYQGLSTATKDTYDALHMQALPPR |
| 01P08A (T2A) | GSGEGRGSLLTCGDVEENPGP |
| SEQ ID NO: 84 | |
| 01P08A (IL-15) | MRISKPHLRSISIQCYLCLLLNSHFLTEAGIHVFILGCFSAGLPKTEA |
| SEQ ID NO: 86 | NWVNVISDLKKIEDLIQSMHIDATLYTESDVHPSCKVTAMKCFLLELQ |
| | VISLESGDASIHDTVENLIILANNSLSSNGNVTESGCKECEELEEKNI |
| | KEFLQSFVHIVQMFINTS |
| 16M16A (CAR | MALPVTALLLPLALLLHAARPQPVLTQPPSASASLGASVTLTCTLSSG |
| with signal peptide) | YSNYKVDWYQQRPGKGPRFVMRVGTGGIVGSKGDGIPDRFSVLGSGLN |
| SEQ ID NO: 101 | RYLTIKNIQEEDESDYHCGADHGSGSNFVYVFGTGTKVTVLGGGGSGG |
| | GGSGGGGSQVQLVQSGTEVKKPGASVKVSCKASGSTFTSFFIHWVRQA |
| | PGQGLEWMGLINPSGGGTGYALKFQGRVTMTRDTSTSTVYMELSSLRS |
| | EDTAVYFCSRGNYEAFDLWGHGTMVTVSSAKPTTTPAPRPPTPAPTIA |
| | SQPLSLRPEACRPAAGGAVHTRGLDFACDFWVLVVVGGVLACYSLLVT |
| | VAFIIFWVRSKRSRLLHSDYMNMTPRRPGPTRKHYQPYAPPRDFAAYR |
| | SERVQPLEENVGNAARPRFERNKRVKFSRSADAPAYQQGQNQLYNELN |
| | LGRREEYDVLDKRRGRDPEMGGKPRRKNPQEGLYNELQKDKMAEAYSE |
| | IGMKGERRRGKGHDGLYQGLSTATKDTYDALHMQALPPR |
| 16M16A (T2A) | GSGEGRGSLLTCGDVEENPGP |
| SEQ ID NO: 84 | |
| 16M16A (IL-15) | MRISKPHLRSISIQCYLCLLLNSHFLTEAGIHVFILGCFSAGLPKTEA |
| SEQ ID NO: 86 | NWVNVISDLKKIEDLIQSMHIDATLYTESDVHPSCKVTAMKCELLELQ |
| | VISLESGDASIHDTVENLIILANNSLSSNGNVTESGCKECEELEEKNI |
| | KEFLQSFVHIVQMFINTS |
| 13I04A (CAR with | MALPVTALLLPLALLLHAARPDIVMTQSPDSLAVSLGERATINCKSSQ |
| signal peptide) | SVLYSSYNKNYLAWYQQKPGQPPKILIYWASTRESGVPDRFTGSGSGT |
| SEQ ID NO: 102 | DFTLTISSLQAEDVAVYYCQQYYSTMYSFGQGTKLEIKGGGGSGGGGS |
| | GGGGSEVQLVESGGGLVQPGGSLRLSCAASGFTSSNYWMSWVRQAPGK |
| | GLEWVANIKHDGSEKYFVDSVKGRFTISRDNANNSLYLQMNSLRDEDT |
| | AVYYCARMRPWYYDLWGRGTLVTVSSAKPTTTPAPRPPTPAPTIASQP |
| | LSLRPEACRPAAGGAVHTRGLDFACDFWVLVVVGGVLACYSLLVTVAF |
| | IIFWVRSKRSRLLHSDYMNMTPRRPGPTRKHYQPYAPPRDFAAYRSER |
| | VQPLEENVGNAARPRFERNKRVKFSRSADAPAYQQGQNQLYNELNLGR |
| | REEYDVLDKRRGRDPEMGGKPRRKNPQEGLYNELQKDKMAEAYSEIGM |
| | KGERRRGKGHDGLYQGLSTATKDTYDALHMQALPPR |
| 13I04A (T2A) | GSGEGRGSLLTCGDVEENPGP |
| SEQ ID NO: 84 | |
| 13I04A (IL-15) | MRISKPHLRSISIQCYLCLLLNSHFLTEAGIHVFILGCFSAGLPKTEA |
| SEQ ID NO: 86 | NWVNVISDLKKIEDLIQSMHIDATLYTESDVHPSCKVTAMKCELLELQ |
| | VISLESGDASIHDTVENLIILANNSLSSNGNVTESGCKECEELEEKNI |
| | KEFLQSFVHIVQMFINTS |
| 13L15A Codon | ATGGCTCTGCCAGTGACTGCACTGCTGCTGCCACTGGCCCTGCTGCTG |
| Optimized (CAR | CACGCAGCTCGACCTCAACCGGTACTCACACAACCACCGTCAGCAAGT |
| with signal peptide) | GCCTCCCTTGGCGCTTCTGTTACCCTCACATGCACCTTGTCTTCTGGC |
| SEQ ID NO: 103 | TACAGCAACTACCGAGTAGATTGGTACCAACAACGACCTGGCAAAGGA |
| | CCTCGGTTCGTCATGAGGGTCGGGACAGGAGGCATTGTCGGGAGCAAA |
| | GGTGACGGAATACCTGACCGATTCTCCGTCTTGGGGAGTGGGCTTAAC |
| | CGCTACTTGACCATCAAGAACATACAAGAAGAAGACGAATCTGACTAT |
| | CATTGTGGTGCGGATCATGGCAGTGGGAGTAACTTCGTTTGGGTTTTT |
| | GGTGGAGGTACAAAACTGACTGTACTTGGAGGCGGTGGATCTGGAGGT |
| | GGAGGTTCCGGAGGGGGCGGATCACAGGCGCAGCTCGTGCAGAGCGGT |
| | GCGGAGGTAAAGCGCCCAGGGGCTAGCGTGAAAGTTTCATGCAAAGCC |
| | TCTGGCTATACGTTTATTAGCTACCTGCTGCACTGGGTTAGGCAAGCG |
| | CCCGGACAAGGATTGGAGTGGATGGTGATAATCAATCCCAGTGTGGGT |
| | TCCACATCATATGCCCAGAAGTTCCAAGGGCGAGTTACCATGACTCGA |
| | GACACCTCCACATCCACCTTGTACATGGAGTTGTCATCCTTGAGATCC |
| | GAAGATACGGCTGTCTACTATTGTGCGCGGTCCAGTCGGAACTATTGG |
| | GACGGAGCTTTCGATATATGGGGCCAAGGGACTATGGTTACTGTGTCT |
| | TCCGCAAAACCTACCACAACTCCTGCACCACGCCCCCCTACTCCAGCA |
| | CCTACCATCGCATCTCAGCCACTGAGTCTGCGACCAGAGGCCTGCCGG |
| | CCCGCCGCCGGCGGGGCCGTCCATACCAGAGGGCTGGACTTTGCCTGC |
| | GATTTTTGGGTCCTGGTGGTCGTGGGAGGGGTGCTGGCATGTTACTCA |
| | CTGCTGGTCACCGTGGCCTTCATCATCTTCTGGGTGCGGAGCAAGAGG |
| | TCCCGCCTGCTGCACAGCGACTATATGAACATGACCCCACGGAGACCG |
| | GGTCCCACTCGCAAACACTACCAGCCCTATGCTCCACCCCGGGACTTC |
| | GCGGCCTACAGGTCAGAGAGAGTGCAGCCCCTGGAAGAGAATGTCGGC |
| | AACGCTGCCCGCCCAAGATTTGAAAGGAACAAACGAGTGAAGTTCAGC |
| | AGGTCCGCCGACGCTCCTGCATACCAGCAGGGACAGAACCAGCTGTAT |
| | AACGAGCTGAATCTGGGCCGGAGAGAGGAATACGACGTGCTGGACAAA |
| | AGGCGGGGCCGGGACCCCGAAATGGGAGGGAAGCCACGACGGAAAAAC |
| | CCCCAGGAGGGCCTGTACAATGAGCTGCAAAAGGACAAAATGGCCGAG |
| | GCTTATTCTGAAATCGGGATGAAGGGAGAGAGAAGGCGCGGAAAAGGC |
| | CACGATGGCCTGTACCAGGGGCTGAGCACCGCTACAAAGGACACCTAT |
| | GATGCACTGCACATGCAGGCCCTGCCCCCTCGG |
| 13L15A Codon | GGCTCAGGTGAGGGGCGCGGGAGCCTGCTGACTTGTGGGGATGTAGAG |
| Optimized (T2A) | GAAAATCCTGGTCCT |
| SEQ ID NO: 85 | |
| 13L15A Codon | ATGAGAATCAGCAAACCACACCTCCGGAGCATATCAATCCAGTGTTAC |
| Optimized (IL-15) | TTGTGCCTTCTTTTGAACTCCCATTTCCTCACCGAGGCAGGCATTCAT |
| SEQ ID NO: 87 | GTGTTCATATTGGGGTGCTTTAGTGCTGGGCTTCCGAAAACGGAAGCT |
| | AACTGGGTAAACGTCATCAGTGACCTTAAAAAAATTGAGGATCTTATC |
| | CAATCAATGCACATCGACGCGACTCTCTACACAGAATCTGACGTACAC |
| | CCGTCATGCAAAGTCACGGCAATGAAGTGTTTTCTTCTCGAGCTCCAA |
| | GTAATTTCCCTGGAGTCTGGCGATGCCTCCATCCACGATACGGTTGAA |
| | AATCTGATTATATTGGCCAACAATAGCCTCAGTTCTAACGGTAACGTG |
| | ACTGAAAGTGGCTGCAAAGAGTGCGAAGAGCTCGAAGAAAAGAATATC |
| | AAGGAGTTCCTCCAATCATTTGTTCACATTGTGCAAATGTTTATCAAC |
| | ACCTCTTGA |
| 13L15A Codon | ATGGCTCTGCCAGTGACTGCACTGCTGCTGCCACTGGCCCTGCTGCTG |
| Optimized | CACGCAGCTCGACCTCAACCGGTACTCACACAACCACCGTCAGCAAGT |
| SEQ ID NO: 104 | GCCTCCCTTGGCGCTTCTGTTACCCTCACATGCACCTTGTCTTCTGGC |
| | TACAGCAACTACCGAGTAGATTGGTACCAACAACGACCTGGCAAAGGA |
| | CCTCGGTTCGTCATGAGGGTCGGGACAGGAGGCATTGTCGGGAGCAAA |
| | GGTGACGGAATACCTGACCGATTCTCCGTCTTGGGGAGTGGGCTTAAC |
| | CGCTACTTGACCATCAAGAACATACAAGAAGAAGACGAATCTGACTAT |
| | CATTGTGGTGCGGATCATGGCAGTGGGAGTAACTTCGTTTGGGTTTTT |
| | GGTGGAGGTACAAAACTGACTGTACTTGGAGGCGGTGGATCTGGAGGT |
| | GGAGGTTCCGGAGGGGGCGGATCACAGGCGCAGCTCGTGCAGAGCGGT |
| | GCGGAGGTAAAGCGCCCAGGGGCTAGCGTGAAAGTTTCATGCAAAGCC |
| | TCTGGCTATACGTTTATTAGCTACCTGCTGCACTGGGTTAGGCAAGCG |
| | CCCGGACAAGGATTGGAGTGGATGGTGATAATCAATCCCAGTGTGGGT |
| | TCCACATCATATGCCCAGAAGTTCCAAGGGCGAGTTACCATGACTCGA |
| | GACACCTCCACATCCACCTTGTACATGGAGTTGTCATCCTTGAGATCC |
| | GAAGATACGGCTGTCTACTATTGTGCGCGGTCCAGTCGGAACTATTGG |
| | GACGGAGCTTTCGATATATGGGGCCAAGGGACTATGGTTACTGTGTCT |
| | TCCGCAAAACCTACCACAACTCCTGCACCACGCCCCCCTACTCCAGCA |
| | CCTACCATCGCATCTCAGCCACTGAGTCTGCGACCAGAGGCCTGCCGG |
| | CCCGCCGCCGGCGGGGCCGTCCATACCAGAGGGCTGGACTTTGCCTGC |
| | GATTTTTGGGTCCTGGTGGTCGTGGGAGGGGTGCTGGCATGTTACTCA |
| | CTGCTGGTCACCGTGGCCTTCATCATCTTCTGGGTGCGGAGCAAGAGG |
| | TCCCGCCTGCTGCACAGCGACTATATGAACATGACCCCACGGAGACCG |
| | GGTCCCACTCGCAAACACTACCAGCCCTATGCTCCACCCCGGGACTTC |
| | GCGGCCTACAGGTCAGAGAGAGTGCAGCCCCTGGAAGAGAATGTCGGC |
| | AACGCTGCCCGCCCAAGATTTGAAAGGAACAAACGAGTGAAGTTCAGC |
| | AGGTCCGCCGACGCTCCTGCATACCAGCAGGGACAGAACCAGCTGTAT |
| | AACGAGCTGAATCTGGGCCGGAGAGAGGAATACGACGTGCTGGACAAA |
| | AGGCGGGGCCGGGACCCCGAAATGGGAGGGAAGCCACGACGGAAAAAC |
| | CCCCAGGAGGGCCTGTACAATGAGCTGCAAAAGGACAAAATGGCCGAG |
| | GCTTATTCTGAAATCGGGATGAAGGGAGAGAGAAGGCGCGGAAAAGGC |
| | CACGATGGCCTGTACCAGGGGCTGAGCACCGCTACAAAGGACACCTAT |
| | GATGCACTGCACATGCAGGCCCTGCCCCCTCGGGGCTCAGGTGAGGGG |
| | CGCGGGAGCCTGCTGACTTGTGGGGATGTAGAGGAAAATCCTGGTCCT |
| | ATGAGAATCAGCAAACCACACCTCCGGAGCATATCAATCCAGTGTTAC |
| | TTGTGCCTTCTTTTGAACTCCCATTTCCTCACCGAGGCAGGCATTCAT |
| | GTGTTCATATTGGGGTGCTTTAGTGCTGGGCTTCCGAAAACGGAAGCT |
| | AACTGGGTAAACGTCATCAGTGACCTTAAAAAAATTGAGGATCTTATC |
| | CAATCAATGCACATCGACGCGACTCTCTACACAGAATCTGACGTACAC |
| | CCGTCATGCAAAGTCACGGCAATGAAGTGTTTTCTTCTCGAGCTCCAA |
| | GTAATTTCCCTGGAGTCTGGCGATGCCTCCATCCACGATACGGTTGAA |
| | AATCTGATTATATTGGCCAACAATAGCCTCAGTTCTAACGGTAACGTG |
| | ACTGAAAGTGGCTGCAAAGAGTGCGAAGAGCTCGAAGAAAAGAATATC |
| | AAGGAGTTCCTCCAATCATTTGTTCACATTGTGCAAATGTTTATCAAC |
| | ACCTCTTGA |
| 14D14A Codon | ATGGCTCTGCCAGTGACTGCACTGCTGCTGCCACTGGCCCTGCTGCTG |
| Optimized (CAR | CACGCAGCTCGACCTGATATTGTCATGACCCAGTCCCCTCTCAGTCTC |
| with signal peptide) | CCGGTAACTCCGGGCGAACCAGCGAGTATTTCTTGTAGGAGTTCCCAG |
| SEQ ID NO: 105 | AGCTTGCTGCATAGCAATGGCTATAATTACTTGGACTGGTACCTGCAA |
| | AAACCCGGCCAGTCACCCCAACTGCTCATCTATTTGGGATCAAACCGG |
| | GCGAGCGGCGTACCCGACAGGTTCAGTGGCAGCGGTTCCGGAACTGAC |
| | TTCACTCTGAAAATTAGTCGCGTAGAAGCCGAGGATGTGGGGGTGTAC |
| | TACTGCCTCCAGGCCCTCCAAACTCCCCTTACTTTCGGCGGTGGTACC |
| | AAAGTGGAGATAAAAGGAGGCGGTGGATCTGGAGGTGGAGGTTCCGGA |
| | GGGGGCGGATCACAGGTCCAGCTGCAGGAAAGTGGTCCTGGCCTTGTA |
| | AAGCCCTCCGAAACACTCTCACTGACATGCACTGTAAGCGGAGGGTCA |
| | ATTTCCTCTTACTACTGGAGCTGGATAAGACAAAGCGCCGGCAAAGGA |
| | CTCGAGTGGATAGGCAGGATGTATACCTCTGGGTCAACGAACTACAAT |
| | CCGTCCCTCAAATCACGCGTAACAATGAGCGTGGACACGTCAAAGAAC |
| | CAATTCTCTTTGAAGCTTACTAGCGTTACGGCCGCAGACACAGCTGTT |
| | TACTATTGCGCGCGCGAACGCGGTTACTATTATGGTGGAGGGGGGTGG |
| | ATTGACCCATGGGGTCAGGGAACCTTGGTCACAGTTTCTTCCGCAAAA |
| | CCTACCACAACTCCTGCACCACGCCCCCCTACTCCAGCACCTACCATC |
| | GCATCTCAGCCACTGAGTCTGCGACCAGAGGCCTGCCGGCCCGCCGCC |
| | GGCGGGGCCGTCCATACCAGAGGGCTGGACTTTGCCTGCGATTTTTGG |
| | GTCCTGGTGGTCGTGGGAGGGGTGCTGGCATGTTACTCACTGCTGGTC |
| | ACCGTGGCCTTCATCATCTTCTGGGTGCGGAGCAAGAGGTCCCGCCTG |
| | CTGCACAGCGACTATATGAACATGACCCCACGGAGACCGGGTCCCACT |
| | CGCAAACACTACCAGCCCTATGCTCCACCCCGGGACTTCGCGGCCTAC |
| | AGGTCAGAGAGAGTGCAGCCCCTGGAAGAGAATGTCGGCAACGCTGCC |
| | CGCCCAAGATTTGAAAGGAACAAACGAGTGAAGTTCAGCAGGTCCGCC |
| | GACGCTCCTGCATACCAGCAGGGACAGAACCAGCTGTATAACGAGCTG |
| | AATCTGGGCCGGAGAGAGGAATACGACGTGCTGGACAAAAGGCGGGGC |
| | CGGGACCCCGAAATGGGAGGGAAGCCACGACGGAAAAACCCCCAGGAG |
| | GGCCTGTACAATGAGCTGCAAAAGGACAAAATGGCCGAGGCTTATTCT |
| | GAAATCGGGATGAAGGGAGAGAGAAGGCGCGGAAAAGGCCACGATGGC |
| | CTGTACCAGGGGCTGAGCACCGCTACAAAGGACACCTATGATGCACTG |
| | CACATGCAGGCCCTGCCCCCTCGG |
| 14D14A Codon | GGCTCAGGTGAGGGGCGCGGGAGCCTGCTGACTTGTGGGGATGTAGAG |
| Optimized (T2A) | GAAAATCCTGGTCCT |
| SEQ ID NO: 85 | |
| 14D14A Codon | ATGAGAATCAGCAAACCACACCTCCGGAGCATATCAATCCAGTGTTAC |
| Optimized (IL-15) | TTGTGCCTTCTTTTGAACTCCCATTTCCTCACCGAGGCAGGCATTCAT |
| SEQ ID NO: 87 | GTGTTCATATTGGGGTGCTTTAGTGCTGGGCTTCCGAAAACGGAAGCT |
| | AACTGGGTAAACGTCATCAGTGACCTTAAAAAAATTGAGGATCTTATC |
| | CAATCAATGCACATCGACGCGACTCTCTACACAGAATCTGACGTACAC |
| | CCGTCATGCAAAGTCACGGCAATGAAGTGTTTTCTTCTCGAGCTCCAA |
| | GTAATTTCCCTGGAGTCTGGCGATGCCTCCATCCACGATACGGTTGAA |
| | AATCTGATTATATTGGCCAACAATAGCCTCAGTTCTAACGGTAACGTG |
| | ACTGAAAGTGGCTGCAAAGAGTGCGAAGAGCTCGAAGAAAAGAATATC |
| | AAGGAGTTCCTCCAATCATTTGTTCACATTGTGCAAATGTTTATCAAC |
| | ACCTCTTGA |
| 14D14A Codon | ATGGCTCTGCCAGTGACTGCACTGCTGCTGCCACTGGCCCTGCTGCTG |
| Optimized | CACGCAGCTCGACCTGATATTGTCATGACCCAGTCCCCTCTCAGTCTC |
| SEQ ID NO: 106 | CCGGTAACTCCGGGCGAACCAGCGAGTATTTCTTGTAGGAGTTCCCAG |
| | AGCTTGCTGCATAGCAATGGCTATAATTACTTGGACTGGTACCTGCAA |
| | AAACCCGGCCAGTCACCCCAACTGCTCATCTATTTGGGATCAAACCGG |
| | GCGAGCGGCGTACCCGACAGGTTCAGTGGCAGCGGTTCCGGAACTGAC |
| | TTCACTCTGAAAATTAGTCGCGTAGAAGCCGAGGATGTGGGGGTGTAC |
| | TACTGCCTCCAGGCCCTCCAAACTCCCCTTACTTTCGGCGGTGGTACC |
| | AAAGTGGAGATAAAAGGAGGCGGTGGATCTGGAGGTGGAGGTTCCGGA |
| | GGGGGCGGATCACAGGTCCAGCTGCAGGAAAGTGGTCCTGGCCTTGTA |
| | AAGCCCTCCGAAACACTCTCACTGACATGCACTGTAAGCGGAGGGTCA |
| | ATTTCCTCTTACTACTGGAGCTGGATAAGACAAAGCGCCGGCAAAGGA |
| | CTCGAGTGGATAGGCAGGATGTATACCTCTGGGTCAACGAACTACAAT |
| | CCGTCCCTCAAATCACGCGTAACAATGAGCGTGGACACGTCAAAGAAC |
| | CAATTCTCTTTGAAGCTTACTAGCGTTACGGCCGCAGACACAGCTGTT |
| | TACTATTGCGCGCGCGAACGCGGTTACTATTATGGTGGAGGGGGGTGG |
| | ATTGACCCATGGGGTCAGGGAACCTTGGTCACAGTTTCTTCCGCAAAA |
| | CCTACCACAACTCCTGCACCACGCCCCCCTACTCCAGCACCTACCATC |
| | GCATCTCAGCCACTGAGTCTGCGACCAGAGGCCTGCCGGCCCGCCGCC |
| | GGCGGGGCCGTCCATACCAGAGGGCTGGACTTTGCCTGCGATTTTTGG |
| | GTCCTGGTGGTCGTGGGAGGGGTGCTGGCATGTTACTCACTGCTGGTC |
| | ACCGTGGCCTTCATCATCTTCTGGGTGCGGAGCAAGAGGTCCCGCCTG |
| | CTGCACAGCGACTATATGAACATGACCCCACGGAGACCGGGTCCCACT |
| | CGCAAACACTACCAGCCCTATGCTCCACCCCGGGACTTCGCGGCCTAC |
| | AGGTCAGAGAGAGTGCAGCCCCTGGAAGAGAATGTCGGCAACGCTGCC |
| | CGCCCAAGATTTGAAAGGAACAAACGAGTGAAGTTCAGCAGGTCCGCC |
| | GACGCTCCTGCATACCAGCAGGGACAGAACCAGCTGTATAACGAGCTG |
| | AATCTGGGCCGGAGAGAGGAATACGACGTGCTGGACAAAAGGCGGGGC |
| | CGGGACCCCGAAATGGGAGGGAAGCCACGACGGAAAAACCCCCAGGAG |
| | GGCCTGTACAATGAGCTGCAAAAGGACAAAATGGCCGAGGCTTATTCT |
| | GAAATCGGGATGAAGGGAGAGAGAAGGCGCGGAAAAGGCCACGATGGC |
| | CTGTACCAGGGGCTGAGCACCGCTACAAAGGACACCTATGATGCACTG |
| | CACATGCAGGCCCTGCCCCCTCGGGGCTCAGGTGAGGGGCGCGGGAGC |
| | CTGCTGACTTGTGGGGATGTAGAGGAAAATCCTGGTCCTATGAGAATC |
| | AGCAAACCACACCTCCGGAGCATATCAATCCAGTGTTACTTGTGCCTT |
| | CTTTTGAACTCCCATTTCCTCACCGAGGCAGGCATTCATGTGTTCATA |
| | TTGGGGTGCTTTAGTGCTGGGCTTCCGAAAACGGAAGCTAACTGGGTA |
| | AACGTCATCAGTGACCTTAAAAAAATTGAGGATCTTATCCAATCAATG |
| | CACATCGACGCGACTCTCTACACAGAATCTGACGTACACCCGTCATGC |
| | AAAGTCACGGCAATGAAGTGTTTTCTTCTCGAGCTCCAAGTAATTTCC |
| | CTGGAGTCTGGCGATGCCTCCATCCACGATACGGTTGAAAATCTGATT |
| | ATATTGGCCAACAATAGCCTCAGTTCTAACGGTAACGTGACTGAAAGT |
| | GGCTGCAAAGAGTGCGAAGAGCTCGAAGAAAAGAATATCAAGGAGTTC |
| | CTCCAATCATTTGTTCACATTGTGCAAATGTTTATCAACACCTCTTGA |
| 01P08A Codon | ATGGCTCTGCCAGTGACTGCACTGCTGCTGCCACTGGCCCTGCTGCTG |
| Optimized (CAR | CACGCAGCTCGACCTCAGCCAGTCTTGACTCAACCACCAAGCGCGTCC |
| with signal peptide) | GCAAGTTTGGGTGCTTCTGTTACTTTGACTTGTACACTCAGCTCTGGT |
| SEQ ID NO: 107 | TATTCCAATTACAAGGTGGACTGGTATCAACAGCGGCCTGGAAAGGGG |
| | CCGAGATTCGTCATGCGAGTAGGCACAGGAGGTATCGTCGGAAGTAAA |
| | GGTGATGGGATCCCAGATCGATTCTCCGTACTGGGGTCCGGTCTGATA |
| | AGGTACCTCACAATAAAGAATATCCAGGAGGAAGATGAGTCCGATTAC |
| | CATTGTGGGGCTGACCATGGCAGCGGCAATAATTTCGTTTACGTATTC |
| | GGAACCGGGACCAAAGTCACGGTTCTCGGAGGCGGTGGATCTGGAGGT |
| | GGAGGTTCCGGAGGGGGCGGATCACAGGTGCAGCTTCAGGAGTCTGGC |
| | CCTGGATTGGTGAAACCTTCCGAAACGCTTAGCTTGACGTGTATCGTC |
| | AGTGGTGGCTCAATTAGCAGCTATTACTGGAATTGGATCCGACAGCCC |
| | GCCGGGAAAGGTCTGGAATGGATAGGGAGAATATACAAAAGCGGGAAC |
| | AACATATATAACCCCAGTTTGCAGTCCAGAGTAACTATGTCTCTCGAT |
| | ACTAGTAAGAACCAGTTCAGCCTTAAACTCAGTTCCGTGACTGCCGCC |
| | GACACGGCGGTGTACTATTGCGCCGACGGAGGGAATTACTTTGACTGG |
| | TTTGACCCGTGGGGTCAGGGCACTCTTGTGATAGTAAGTTCCGCAAAA |
| | CCTACCACAACTCCTGCACCACGCCCCCCTACTCCAGCACCTACCATC |
| | GCATCTCAGCCACTGAGTCTGCGACCAGAGGCCTGCCGGCCCGCCGCC |
| | GGCGGGGCCGTCCATACCAGAGGGCTGGACTTTGCCTGCGATTTTTGG |
| | GTCCTGGTGGTCGTGGGAGGGGTGCTGGCATGTTACTCACTGCTGGTC |
| | ACCGTGGCCTTCATCATCTTCTGGGTGCGGAGCAAGAGGTCCCGCCTG |
| | CTGCACAGCGACTATATGAACATGACCCCACGGAGACCGGGTCCCACT |
| | CGCAAACACTACCAGCCCTATGCTCCACCCCGGGACTTCGCGGCCTAC |
| | AGGTCAGAGAGAGTGCAGCCCCTGGAAGAGAATGTCGGCAACGCTGCC |
| | CGCCCAAGATTTGAAAGGAACAAACGAGTGAAGTTCAGCAGGTCCGCC |
| | GACGCTCCTGCATACCAGCAGGGACAGAACCAGCTGTATAACGAGCTG |
| | AATCTGGGCCGGAGAGAGGAATACGACGTGCTGGACAAAAGGCGGGGC |
| | CGGGACCCCGAAATGGGAGGGAAGCCACGACGGAAAAACCCCCAGGAG |
| | GGCCTGTACAATGAGCTGCAAAAGGACAAAATGGCCGAGGCTTATTCT |
| | GAAATCGGGATGAAGGGAGAGAGAAGGCGCGGAAAAGGCCACGATGGC |
| | CTGTACCAGGGGCTGAGCACCGCTACAAAGGACACCTATGATGCACTG |
| | CACATGCAGGCCCTGCCCCCTCGG |
| 01P08A Codon | GGCTCAGGTGAGGGGCGCGGGAGCCTGCTGACTTGTGGGGATGTAGAG |
| Optimized (T2A) | GAAAATCCTGGTCCT |
| SEQ ID NO: 85 | |
| 01P08A Codon | ATGAGAATCAGCAAACCACACCTCCGGAGCATATCAATCCAGTGTTAC |
| Optimized (IL-15) | TTGTGCCTTCTTTTGAACTCCCATTTCCTCACCGAGGCAGGCATTCAT |
| SEQ ID NO: 87 | GTGTTCATATTGGGGTGCTTTAGTGCTGGGCTTCCGAAAACGGAAGCT |
| | AACTGGGTAAACGTCATCAGTGACCTTAAAAAAATTGAGGATCTTATC |
| | CAATCAATGCACATCGACGCGACTCTCTACACAGAATCTGACGTACAC |
| | CCGTCATGCAAAGTCACGGCAATGAAGTGTTTTCTTCTCGAGCTCCAA |
| | GTAATTTCCCTGGAGTCTGGCGATGCCTCCATCCACGATACGGTTGAA |
| | AATCTGATTATATTGGCCAACAATAGCCTCAGTTCTAACGGTAACGTG |
| | ACTGAAAGTGGCTGCAAAGAGTGCGAAGAGCTCGAAGAAAAGAATATC |
| | AAGGAGTTCCTCCAATCATTTGTTCACATTGTGCAAATGTTTATCAAC |
| | ACCTCTTGA |
| 01P08A Codon | ATGGCTCTGCCAGTGACTGCACTGCTGCTGCCACTGGCCCTGCTGCTG |
| Optimized | CACGCAGCTCGACCTCAGCCAGTCTTGACTCAACCACCAAGCGCGTCC |
| SEQ ID NO: 108 | GCAAGTTTGGGTGCTTCTGTTACTTTGACTTGTACACTCAGCTCTGGT |
| | TATTCCAATTACAAGGTGGACTGGTATCAACAGCGGCCTGGAAAGGGG |
| | CCGAGATTCGTCATGCGAGTAGGCACAGGAGGTATCGTCGGAAGTAAA |
| | GGTGATGGGATCCCAGATCGATTCTCCGTACTGGGGTCCGGTCTGATA |
| | AGGTACCTCACAATAAAGAATATCCAGGAGGAAGATGAGTCCGATTAC |
| | CATTGTGGGGCTGACCATGGCAGCGGCAATAATTTCGTTTACGTATTC |
| | GGAACCGGGACCAAAGTCACGGTTCTCGGAGGCGGTGGATCTGGAGGT |
| | GGAGGTTCCGGAGGGGGCGGATCACAGGTGCAGCTTCAGGAGTCTGGC |
| | CCTGGATTGGTGAAACCTTCCGAAACGCTTAGCTTGACGTGTATCGTC |
| | AGTGGTGGCTCAATTAGCAGCTATTACTGGAATTGGATCCGACAGCCC |
| | GCCGGGAAAGGTCTGGAATGGATAGGGAGAATATACAAAAGCGGGAAC |
| | AACATATATAACCCCAGTTTGCAGTCCAGAGTAACTATGTCTCTCGAT |
| | ACTAGTAAGAACCAGTTCAGCCTTAAACTCAGTTCCGTGACTGCCGCC |
| | GACACGGCGGTGTACTATTGCGCCGACGGAGGGAATTACTTTGACTGG |
| | TTTGACCCGTGGGGTCAGGGCACTCTTGTGATAGTAAGTTCCGCAAAA |
| | CCTACCACAACTCCTGCACCACGCCCCCCTACTCCAGCACCTACCATC |
| | GCATCTCAGCCACTGAGTCTGCGACCAGAGGCCTGCCGGCCCGCCGCC |
| | GGCGGGGCCGTCCATACCAGAGGGCTGGACTTTGCCTGCGATTTTTGG |
| | GTCCTGGTGGTCGTGGGAGGGGTGCTGGCATGTTACTCACTGCTGGTC |
| | ACCGTGGCCTTCATCATCTTCTGGGTGCGGAGCAAGAGGTCCCGCCTG |
| | CTGCACAGCGACTATATGAACATGACCCCACGGAGACCGGGTCCCACT |
| | CGCAAACACTACCAGCCCTATGCTCCACCCCGGGACTTCGCGGCCTAC |
| | AGGTCAGAGAGAGTGCAGCCCCTGGAAGAGAATGTCGGCAACGCTGCC |
| | CGCCCAAGATTTGAAAGGAACAAACGAGTGAAGTTCAGCAGGTCCGCC |
| | GACGCTCCTGCATACCAGCAGGGACAGAACCAGCTGTATAACGAGCTG |
| | AATCTGGGCCGGAGAGAGGAATACGACGTGCTGGACAAAAGGCGGGGC |
| | CGGGACCCCGAAATGGGAGGGAAGCCACGACGGAAAAACCCCCAGGAG |
| | GGCCTGTACAATGAGCTGCAAAAGGACAAAATGGCCGAGGCTTATTCT |
| | GAAATCGGGATGAAGGGAGAGAGAAGGCGCGGAAAAGGCCACGATGGC |
| | CTGTACCAGGGGCTGAGCACCGCTACAAAGGACACCTATGATGCACTG |
| | CACATGCAGGCCCTGCCCCCTCGGGGCTCAGGTGAGGGGCGCGGGAGC |
| | CTGCTGACTTGTGGGGATGTAGAGGAAAATCCTGGTCCTATGAGAATC |
| | AGCAAACCACACCTCCGGAGCATATCAATCCAGTGTTACTTGTGCCTT |
| | CTTTTGAACTCCCATTTCCTCACCGAGGCAGGCATTCATGTGTTCATA |
| | TTGGGGTGCTTTAGTGCTGGGCTTCCGAAAACGGAAGCTAACTGGGTA |
| | AACGTCATCAGTGACCTTAAAAAAATTGAGGATCTTATCCAATCAATG |
| | CACATCGACGCGACTCTCTACACAGAATCTGACGTACACCCGTCATGC |
| | AAAGTCACGGCAATGAAGTGTTTTCTTCTCGAGCTCCAAGTAATTTCC |
| | CTGGAGTCTGGCGATGCCTCCATCCACGATACGGTTGAAAATCTGATT |
| | ATATTGGCCAACAATAGCCTCAGTTCTAACGGTAACGTGACTGAAAGT |
| | GGCTGCAAAGAGTGCGAAGAGCTCGAAGAAAAGAATATCAAGGAGTTC |
| | CTCCAATCATTTGTTCACATTGTGCAAATGTTTATCAACACCTCTTGA |
| 16M16A Codon | ATGGCTCTGCCAGTGACTGCACTGCTGCTGCCACTGGCCCTGCTGCTG |
| Optimized (CAR | CACGCAGCTCGACCTCAACCTGTGCTGACACAGCCTCCTTCTGCGTCT |
| with signal peptide) | GCGTCACTGGGAGCTTCCGTAACACTGACCTGTACGCTCTCTTCTGGA |
| SEQ ID NO: 109 | TATAGCAATTATAAAGTAGACTGGTACCAACAGAGACCGGGGAAGGGG |
| | CCGCGATTCGTTATGAGAGTTGGTACCGGTGGGATCGTCGGATCAAAA |
| | GGGGACGGGATCCCGGATCGATTTTCCGTTCTTGGGTCCGGTCTCAAT |
| | AGATATCTGACAATTAAAAATATACAAGAAGAAGATGAGAGCGACTAC |
| | CATTGCGGAGCGGACCATGGTAGTGGCAGCAACTTTGTCTACGTTTTC |
| | GGGACCGGCACAAAGGTCACCGTACTGGGAGGCGGTGGATCTGGAGGT |
| | GGAGGTTCCGGAGGGGGCGGATCACAGGTCCAGCTCGTTCAGTCTGGA |
| | ACCGAGGTGAAGAAGCCAGGTGCATCCGTTAAAGTGTCTTGTAAAGCC |
| | TCCGGCTCTACATTCACGAGTTTCTTTATACACTGGGTACGCCAGGCG |
| | CCTGGCCAGGGTCTTGAATGGATGGGCCTTATTAATCCATCCGGTGGA |
| | GGTACAGGCTATGCACTTAAGTTCCAAGGCAGGGTCACCATGACGCGC |
| | GACACGTCAACATCAACTGTGTATATGGAACTCTCTTCTCTTCGCTCT |
| | GAAGACACGGCTGTTTATTTCTGCAGTCGAGGAAATTACGAGGCATTC |
| | GACCTTTGGGGGCACGGAACTATGGTGACAGTAAGCTCAGCAAAACCT |
| | ACCACAACTCCTGCACCACGCCCCCCTACTCCAGCACCTACCATCGCA |
| | TCTCAGCCACTGAGTCTGCGACCAGAGGCCTGCCGGCCCGCCGCCGGC |
| | GGGGCCGTCCATACCAGAGGGCTGGACTTTGCCTGCGATTTTTGGGTC |
| | CTGGTGGTCGTGGGAGGGGTGCTGGCATGTTACTCACTGCTGGTCACC |
| | GTGGCCTTCATCATCTTCTGGGTGCGGAGCAAGAGGTCCCGCCTGCTG |
| | CACAGCGACTATATGAACATGACCCCACGGAGACCGGGTCCCACTCGC |
| | AAACACTACCAGCCCTATGCTCCACCCCGGGACTTCGCGGCCTACAGG |
| | TCAGAGAGAGTGCAGCCCCTGGAAGAGAATGTCGGCAACGCTGCCCGC |
| | CCAAGATTTGAAAGGAACAAACGAGTGAAGTTCAGCAGGTCCGCCGAC |
| | GCTCCTGCATACCAGCAGGGACAGAACCAGCTGTATAACGAGCTGAAT |
| | CTGGGCCGGAGAGAGGAATACGACGTGCTGGACAAAAGGCGGGGCCGG |
| | GACCCCGAAATGGGAGGGAAGCCACGACGGAAAAACCCCCAGGAGGGC |
| | CTGTACAATGAGCTGCAAAAGGACAAAATGGCCGAGGCTTATTCTGAA |
| | ATCGGGATGAAGGGAGAGAGAAGGCGCGGAAAAGGCCACGATGGCCTG |
| | TACCAGGGGCTGAGCACCGCTACAAAGGACACCTATGATGCACTGCAC |
| | ATGCAGGCCCTGCCCCCTCGG |
| 16M16A Codon | GGCTCAGGTGAGGGGCGCGGGAGCCTGCTGACTTGTGGGGATGTAGAG |
| Optimized (T2A) | GAAAATCCTGGTCCT |
| SEQ ID NO: 85 | |
| 16M16A Codon | ATGAGAATCAGCAAACCACACCTCCGGAGCATATCAATCCAGTGTTAC |
| Optimized (IL-15) | TTGTGCCTTCTTTTGAACTCCCATTTCCTCACCGAGGCAGGCATTCAT |
| SEQ ID NO: 87 | GTGTTCATATTGGGGTGCTTTAGTGCTGGGCTTCCGAAAACGGAAGCT |
| | AACTGGGTAAACGTCATCAGTGACCTTAAAAAAATTGAGGATCTTATC |
| | CAATCAATGCACATCGACGCGACTCTCTACACAGAATCTGACGTACAC |
| | CCGTCATGCAAAGTCACGGCAATGAAGTGTTTTCTTCTCGAGCTCCAA |
| | GTAATTTCCCTGGAGTCTGGCGATGCCTCCATCCACGATACGGTTGAA |
| | AATCTGATTATATTGGCCAACAATAGCCTCAGTTCTAACGGTAACGTG |
| | ACTGAAAGTGGCTGCAAAGAGTGCGAAGAGCTCGAAGAAAAGAATATC |
| | AAGGAGTTCCTCCAATCATTTGTTCACATTGTGCAAATGTTTATCAAC |
| | ACCTCTTGA |
| 16M16A Codon | ATGGCTCTGCCAGTGACTGCACTGCTGCTGCCACTGGCCCTGCTGCTG |
| Optimized | CACGCAGCTCGACCTCAACCTGTGCTGACACAGCCTCCTTCTGCGTCT |
| SEQ ID NO: 110 | GCGTCACTGGGAGCTTCCGTAACACTGACCTGTACGCTCTCTTCTGGA |
| | TATAGCAATTATAAAGTAGACTGGTACCAACAGAGACCGGGGAAGGGG |
| | CCGCGATTCGTTATGAGAGTTGGTACCGGTGGGATCGTCGGATCAAAA |
| | GGGGACGGGATCCCGGATCGATTTTCCGTTCTTGGGTCCGGTCTCAAT |
| | AGATATCTGACAATTAAAAATATACAAGAAGAAGATGAGAGCGACTAC |
| | CATTGCGGAGCGGACCATGGTAGTGGCAGCAACTTTGTCTACGTTTTC |
| | GGGACCGGCACAAAGGTCACCGTACTGGGAGGCGGTGGATCTGGAGGT |
| | GGAGGTTCCGGAGGGGGCGGATCACAGGTCCAGCTCGTTCAGTCTGGA |
| | ACCGAGGTGAAGAAGCCAGGTGCATCCGTTAAAGTGTCTTGTAAAGCC |
| | TCCGGCTCTACATTCACGAGTTTCTTTATACACTGGGTACGCCAGGCG |
| | CCTGGCCAGGGTCTTGAATGGATGGGCCTTATTAATCCATCCGGTGGA |
| | GGTACAGGCTATGCACTTAAGTTCCAAGGCAGGGTCACCATGACGCGC |
| | GACACGTCAACATCAACTGTGTATATGGAACTCTCTTCTCTTCGCTCT |
| | GAAGACACGGCTGTTTATTTCTGCAGTCGAGGAAATTACGAGGCATTC |
| | GACCTTTGGGGGCACGGAACTATGGTGACAGTAAGCTCAGCAAAACCT |
| | ACCACAACTCCTGCACCACGCCCCCCTACTCCAGCACCTACCATCGCA |
| | TCTCAGCCACTGAGTCTGCGACCAGAGGCCTGCCGGCCCGCCGCCGGC |
| | GGGGCCGTCCATACCAGAGGGCTGGACTTTGCCTGCGATTTTTGGGTC |
| | CTGGTGGTCGTGGGAGGGGTGCTGGCATGTTACTCACTGCTGGTCACC |
| | GTGGCCTTCATCATCTTCTGGGTGCGGAGCAAGAGGTCCCGCCTGCTG |
| | CACAGCGACTATATGAACATGACCCCACGGAGACCGGGTCCCACTCGC |
| | AAACACTACCAGCCCTATGCTCCACCCCGGGACTTCGCGGCCTACAGG |
| | TCAGAGAGAGTGCAGCCCCTGGAAGAGAATGTCGGCAACGCTGCCCGC |
| | CCAAGATTTGAAAGGAACAAACGAGTGAAGTTCAGCAGGTCCGCCGAC |
| | GCTCCTGCATACCAGCAGGGACAGAACCAGCTGTATAACGAGCTGAAT |
| | CTGGGCCGGAGAGAGGAATACGACGTGCTGGACAAAAGGCGGGGCCGG |
| | GACCCCGAAATGGGAGGGAAGCCACGACGGAAAAACCCCCAGGAGGGC |
| | CTGTACAATGAGCTGCAAAAGGACAAAATGGCCGAGGCTTATTCTGAA |
| | ATCGGGATGAAGGGAGAGAGAAGGCGCGGAAAAGGCCACGATGGCCTG |
| | TACCAGGGGCTGAGCACCGCTACAAAGGACACCTATGATGCACTGCAC |
| | ATGCAGGCCCTGCCCCCTCGGGGCTCAGGTGAGGGGCGCGGGAGCCTG |
| | CTGACTTGTGGGGATGTAGAGGAAAATCCTGGTCCTATGAGAATCAGC |
| | AAACCACACCTCCGGAGCATATCAATCCAGTGTTACTTGTGCCTTCTT |
| | TTGAACTCCCATTTCCTCACCGAGGCAGGCATTCATGTGTTCATATTG |
| | GGGTGCTTTAGTGCTGGGCTTCCGAAAACGGAAGCTAACTGGGTAAAC |
| | GTCATCAGTGACCTTAAAAAAATTGAGGATCTTATCCAATCAATGCAC |
| | ATCGACGCGACTCTCTACACAGAATCTGACGTACACCCGTCATGCAAA |
| | GTCACGGCAATGAAGTGTTTTCTTCTCGAGCTCCAAGTAATTTCCCTG |
| | GAGTCTGGCGATGCCTCCATCCACGATACGGTTGAAAATCTGATTATA |
| | TTGGCCAACAATAGCCTCAGTTCTAACGGTAACGTGACTGAAAGTGGC |
| | TGCAAAGAGTGCGAAGAGCTCGAAGAAAAGAATATCAAGGAGTTCCTC |
| | CAATCATTTGTTCACATTGTGCAAATGTTTATCAACACCTCTTGA |
| 13I04A Codon | ATGGCTCTGCCAGTGACTGCACTGCTGCTGCCACTGGCCCTGCTGCTG |
| Optimized (CAR | CACGCAGCTCGACCTGATATAGTCATGACCCAAAGCCCAGATTCCCTT |
| with signal peptide) | GCGGTATCCCTGGGTGAGAGAGCTACGATCAACTGCAAGAGCTCACAA |
| SEQ ID NO: 111 | AGTGTCCTCTACTCCAGCTATAATAAGAATTACTTGGCGTGGTATCAG |
| | CAGAAACCTGGACAGCCACCAAAAATTCTCATTTACTGGGCTTCCACT |
| | CGGGAGAGCGGAGTCCCGGACCGATTCACAGGTTCCGGTTCTGGAACC |
| | GACTTTACCCTGACTATTTCCTCCTTGCAGGCGGAAGACGTTGCTGTG |
| | TACTATTGCCAACAATATTACTCTACAATGTACAGTTTTGGGCAGGGC |
| | ACCAAACTGGAAATCAAGGGAGGCGGTGGATCTGGAGGTGGAGGTTCC |
| | GGAGGGGGCGGATCAGAAGTCCAATTGGTTGAATCCGGCGGAGGGCTG |
| | GTCCAACCGGGTGGATCACTTAGACTTAGCTGTGCAGCGAGCGGCTTT |
| | ACGTCAAGTAACTATTGGATGTCCTGGGTTCGACAAGCACCAGGCAAA |
| | GGATTGGAATGGGTAGCAAACATCAAACATGACGGCAGCGAAAAATAT |
| | TTCGTTGACTCCGTCAAGGGAAGGTTTACAATATCACGCGACAATGCT |
| | AACAACTCTCTCTACCTTCAAATGAATTCTCTCCGAGATGAAGACACA |
| | GCCGTCTACTATTGCGCAAGAATGAGACCTTGGTACTATGACTTGTGG |
| | GGCCGAGGAACGCTCGTGACCGTTTCAAGTGCAAAACCTACCACAACT |
| | CCTGCACCACGCCCCCCTACTCCAGCACCTACCATCGCATCTCAGCCA |
| | CTGAGTCTGCGACCAGAGGCCTGCCGGCCCGCCGCCGGCGGGGCCGTC |
| | CATACCAGAGGGCTGGACTTTGCCTGCGATTTTTGGGTCCTGGTGGTC |
| | GTGGGAGGGGTGCTGGCATGTTACTCACTGCTGGTCACCGTGGCCTTC |
| | ATCATCTTCTGGGTGCGGAGCAAGAGGTCCCGCCTGCTGCACAGCGAC |
| | TATATGAACATGACCCCACGGAGACCGGGTCCCACTCGCAAACACTAC |
| | CAGCCCTATGCTCCACCCCGGGACTTCGCGGCCTACAGGTCAGAGAGA |
| | GTGCAGCCCCTGGAAGAGAATGTCGGCAACGCTGCCCGCCCAAGATTT |
| | GAAAGGAACAAACGAGTGAAGTTCAGCAGGTCCGCCGACGCTCCTGCA |
| | TACCAGCAGGGACAGAACCAGCTGTATAACGAGCTGAATCTGGGCCGG |
| | AGAGAGGAATACGACGTGCTGGACAAAAGGCGGGGCCGGGACCCCGAA |
| | ATGGGAGGGAAGCCACGACGGAAAAACCCCCAGGAGGGCCTGTACAAT |
| | GAGCTGCAAAAGGACAAAATGGCCGAGGCTTATTCTGAAATCGGGATG |
| | AAGGGAGAGAGAAGGCGCGGAAAAGGCCACGATGGCCTGTACCAGGGG |
| | CTGAGCACCGCTACAAAGGACACCTATGATGCACTGCACATGCAGGCC |
| | CTGCCCCCTCGG |
| 13I04A Codon | GGCTCAGGTGAGGGGCGCGGGAGCCTGCTGACTTGTGGGGATGTAGAG |
| Optimized (T2A) | GAAAATCCTGGTCCT |
| SEQ ID NO: 85 | |
| 13I04A Codon | ATGAGAATCAGCAAACCACACCTCCGGAGCATATCAATCCAGTGTTAC |
| Optimized (IL-15) | TTGTGCCTTCTTTTGAACTCCCATTTCCTCACCGAGGCAGGCATTCAT |
| SEQ ID NO: 87 | GTGTTCATATTGGGGTGCTTTAGTGCTGGGCTTCCGAAAACGGAAGCT |
| | AACTGGGTAAACGTCATCAGTGACCTTAAAAAAATTGAGGATCTTATC |
| | CAATCAATGCACATCGACGCGACTCTCTACACAGAATCTGACGTACAC |
| | CCGTCATGCAAAGTCACGGCAATGAAGTGTTTTCTTCTCGAGCTCCAA |
| | GTAATTTCCCTGGAGTCTGGCGATGCCTCCATCCACGATACGGTTGAA |
| | AATCTGATTATATTGGCCAACAATAGCCTCAGTTCTAACGGTAACGTG |
| | ACTGAAAGTGGCTGCAAAGAGTGCGAAGAGCTCGAAGAAAAGAATATC |
| | AAGGAGTTCCTCCAATCATTTGTTCACATTGTGCAAATGTTTATCAAC |
| | ACCTCTTGA |
| 13I04A Codon | ATGGCTCTGCCAGTGACTGCACTGCTGCTGCCACTGGCCCTGCTGCTG |
| Optimized | CACGCAGCTCGACCTGATATAGTCATGACCCAAAGCCCAGATTCCCTT |
| SEQ ID NO: 112 | GCGGTATCCCTGGGTGAGAGAGCTACGATCAACTGCAAGAGCTCACAA |
| | AGTGTCCTCTACTCCAGCTATAATAAGAATTACTTGGCGTGGTATCAG |
| | CAGAAACCTGGACAGCCACCAAAAATTCTCATTTACTGGGCTTCCACT |
| | CGGGAGAGCGGAGTCCCGGACCGATTCACAGGTTCCGGTTCTGGAACC |
| | GACTTTACCCTGACTATTTCCTCCTTGCAGGCGGAAGACGTTGCTGTG |
| | TACTATTGCCAACAATATTACTCTACAATGTACAGTTTTGGGCAGGGC |
| | ACCAAACTGGAAATCAAGGGAGGCGGTGGATCTGGAGGTGGAGGTTCC |
| | GGAGGGGGCGGATCAGAAGTCCAATTGGTTGAATCCGGCGGAGGGCTG |
| | GTCCAACCGGGTGGATCACTTAGACTTAGCTGTGCAGCGAGCGGCTTT |
| | ACGTCAAGTAACTATTGGATGTCCTGGGTTCGACAAGCACCAGGCAAA |
| | GGATTGGAATGGGTAGCAAACATCAAACATGACGGCAGCGAAAAATAT |
| | TTCGTTGACTCCGTCAAGGGAAGGTTTACAATATCACGCGACAATGCT |
| | AACAACTCTCTCTACCTTCAAATGAATTCTCTCCGAGATGAAGACACA |
| | GCCGTCTACTATTGCGCAAGAATGAGACCTTGGTACTATGACTTGTGG |
| | GGCCGAGGAACGCTCGTGACCGTTTCAAGTGCAAAACCTACCACAACT |
| | CCTGCACCACGCCCCCCTACTCCAGCACCTACCATCGCATCTCAGCCA |
| | CTGAGTCTGCGACCAGAGGCCTGCCGGCCCGCCGCCGGCGGGGCCGTC |
| | CATACCAGAGGGCTGGACTTTGCCTGCGATTTTTGGGTCCTGGTGGTC |
| | GTGGGAGGGGTGCTGGCATGTTACTCACTGCTGGTCACCGTGGCCTTC |
| | ATCATCTTCTGGGTGCGGAGCAAGAGGTCCCGCCTGCTGCACAGCGAC |
| | TATATGAACATGACCCCACGGAGACCGGGTCCCACTCGCAAACACTAC |
| | CAGCCCTATGCTCCACCCCGGGACTTCGCGGCCTACAGGTCAGAGAGA |
| | GTGCAGCCCCTGGAAGAGAATGTCGGCAACGCTGCCCGCCCAAGATTT |
| | GAAAGGAACAAACGAGTGAAGTTCAGCAGGTCCGCCGACGCTCCTGCA |
| | TACCAGCAGGGACAGAACCAGCTGTATAACGAGCTGAATCTGGGCCGG |
| | AGAGAGGAATACGACGTGCTGGACAAAAGGCGGGGCCGGGACCCCGAA |
| | ATGGGAGGGAAGCCACGACGGAAAAACCCCCAGGAGGGCCTGTACAAT |
| | GAGCTGCAAAAGGACAAAATGGCCGAGGCTTATTCTGAAATCGGGATG |
| | AAGGGAGAGAGAAGGCGCGGAAAAGGCCACGATGGCCTGTACCAGGGG |
| | CTGAGCACCGCTACAAAGGACACCTATGATGCACTGCACATGCAGGCC |
| | CTGCCCCCTCGGGGCTCAGGTGAGGGGCGCGGGAGCCTGCTGACTTGT |
| | GGGGATGTAGAGGAAAATCCTGGTCCTATGAGAATCAGCAAACCACAC |
| | CTCCGGAGCATATCAATCCAGTGTTACTTGTGCCTTCTTTTGAACTCC |
| | CATTTCCTCACCGAGGCAGGCATTCATGTGTTCATATTGGGGTGCTTT |
| | AGTGCTGGGCTTCCGAAAACGGAAGCTAACTGGGTAAACGTCATCAGT |
| | GACCTTAAAAAAATTGAGGATCTTATCCAATCAATGCACATCGACGCG |
| | ACTCTCTACACAGAATCTGACGTACACCCGTCATGCAAAGTCACGGCA |
| | ATGAAGTGTTTTCTTCTCGAGCTCCAAGTAATTTCCCTGGAGTCTGGC |
| | GATGCCTCCATCCACGATACGGTTGAAAATCTGATTATATTGGCCAAC |
| | AATAGCCTCAGTTCTAACGGTAACGTGACTGAAAGTGGCTGCAAAGAG |
| | TGCGAAGAGCTCGAAGAAAAGAATATCAAGGAGTTCCTCCAATCATTT |
| | GTTCACATTGTGCAAATGTTTATCAACACCTCTTGA |

### Example 6: NK-CARs

Umbilical cord blood NK cells were transduced with lentiviral vectors encoding the CARs described in Example 5.

### 1. Anti-BCMA CAR NKPhenotypic Characterization

The purity as well as expression of antibody-engaging CD16 and activating, inhibitory, and chemokine receptors of multiple batches of anti-BCMA CAR NK cells were measured via flow cytometry. As shown in FIG. 4, the resulting CAR-NK cells expressed both high affinity CD16 and low levels of CD38.

Anti-BCMA CAR NK cell purity was measured using cell surface markers: anti-BCMA CAR NK batches were seen to comprise >99% CD3-CD56+NK cells and <0.1% CD3+, CD14+ and CD19+ cells. CD16 expression of anti-BCMA CAR NK cells was measured. 90.35±2.69% of anti-BCMA CAR NK cells were CD16+. 85±11.44% of anti-BCMA CAR NK cells were BCMA CAR+. NK cells are known to express various NK specific activating and inhibitory receptors. For the various anti-BCMA CAR NK batches that were tested, >80% of cells expressed CD16, NKG2A, NKG2D, NKp30, Tim-3, 50~70% of cells expressed NKp46. DNAM-1, approximately 20% of cells expressed NKG2C, and less than 5% of cells expressed other activating inhibitory receptors.

The purity of anti-BCMA CAR NK cells is represented as CD3-CD56+ cells for NK cells, CD3+ cells for T-cells, CD14+ cells for monocytes and CD19+ cells for B-cells. Five different anti-BCMA CAR NK clones along with non-transduced (NT) CB NK were measured for purity. The results showed 97.6±0.49% (mean±SD) for CD3-CD56+ cells, 1.24±0.79% for CD3+ cells (FIG. 4, Table 13). Therefore, it was confirmed that anti-BCMA CAR NK cells are composed of highly pure NK cell populations, and the other types of cells as impurities were rarely present.

TABLE 13
Cell Purity

| | anti-BCMA CAR NK pre-clinical batch#1 | | | | | |
|---|---|---|---|---|---|---|
| Marker | NT | 13L15A | 14D14A | 01P08A | 16M16A | 13I04A |
| CD3−CD56+ | 98.4 | 98 | 97.4 | 97.4 | 97 | 97.6 |
| (%) | | | | | | |
| CD3+ (%) | 0.19 | 1.15 | 1.69 | 2.28 | 1.67 | 0.5 |

### Comparison of NK Cell Receptors of Anti-BCMA CAR NK Cells

NK cells expressing the five anti-BCMA CARs along with non-transduced (NT) expanded CBNK were also utilized to assess the expression of various NK cell receptors on the final, expanded cell product. It was observed that several NK cell and activating receptors such as CD16, NKG2D, NKG2C, NKp3, NKp46, and DNAM-1 were highly expressed by the final cell product. (FIG. 4. Table 1).

TABLE 14
Cell Receptor Expression

| | anti-BCMA CAR NK pre-clinical batch#1 | | | | | |
|---|---|---|---|---|---|---|
| Marker | NT | 13L15A | 14D14A | 01P08A | 16M16A | 13I04A |
| BCMA CAR | 0.22 | 89 | 75.4 | 92.9 | 97.1 | 70.6 |
| CD16 | 93.1 | 86.2 | 92.6 | 89.1 | 89 | 92.1 |
| CD38 | 3.05 | 30.9 | 15.8 | 30.3 | 27.5 | 21.5 |
| CD57 | 9.98 | 9.11 | 14.2 | 15.4 | 10.6 | 9.11 |
| DNAM-1 | 72.7 | 96.6 | 93.4 | 98 | 98.7 | 93.6 |
| KIR2DL1 | 11.1 | 13.4 | 15.9 | 17 | 14.5 | 14.2 |
| KIR2DL2 | 52.9 | 28.9 | 30.9 | 22.4 | 26.3 | 20.4 |
| KIR3DL1 | 61 | 40.6 | 41.9 | 38.3 | 50.7 | 36.9 |
| KIR3DL2 | 0.01 | 0.096 | 0.085 | 0.23 | 0.099 | 0.069 |
| NKG2A | 47.6 | 75.3 | 85.3 | 82.6 | 75.9 | 83.1 |
| NKG2C | 31.8 | 28.2 | 18.2 | 28.6 | 34.1 | 30.4 |
| NKG2D | 96.1 | 92.3 | 93.5 | 96.4 | 95.6 | 92.2 |
| NKp30 | 97.7 | 86.2 | 96.3 | 95.2 | 83 | 92.1 |
| NKp46 | 86.2 | 60.7 | 84.7 | 70.4 | 67 | 77.9 |
| PD-1 | 0.01 | 0.32 | 0.72 | 0.7 | 0.22 | 0.14 |
| TIGIT | 81.9 | 71.6 | 72.8 | 83.1 | 73.1 | 78 |
| TIM-3 | 92.9 | 91.3 | 93.5 | 96.8 | 95.7 | 96.5 |

### CONCLUSION

The use of surface marker analysis supported the identity and purity and batch-to-batch consistency of the anti-BCMA CAR NK product. Further, extensive assessment of NK-specific activating and inhibitory cell surface markers established the consistent profile of the anti-BCMA CAR NK product post manufacturing expansion process. It is known that CB derived NK cells have immature phenotype such as high expression of NKG2A and low expression of NKG2C. CD62L, CD57, IL-2R. CD16, DNAM-1 comparing to peripheral blood (PB) derived NK cells, and it is also known that CB derived NK cells with the immature phenotypes exhibit low cytotoxicity against tumor cells. Data from this study shows that anti-BCMA CAR NK, an allogeneic cord blood (CB) derived NK cell product, expresses high levels of major activating receptors indicative of potential higher cytotoxicity against tumor cells.

### 2. Potency (Cytotoxicity of Anti-BCMA CAR NKDP Cells to Target Cells)

Cytotoxicity of anti-BCMA CAR NK cells against tumor cell lines was assessed using short (4 hr) and long-term (up to 3 day) extracellular cytotoxicity assays. Cytotoxicity of NK cells can be quantitatively measured at a range of NK cell (effector) to tumor cell (target) ratios. Target cells included NCI-H929 (BCMA-high), Daudi (BCMA-med), and NALM-6 (BCMA-neg), which are cancer cell lines of multiple myeloma, Burkitt's Lymphoma, and leukemia, respectively.

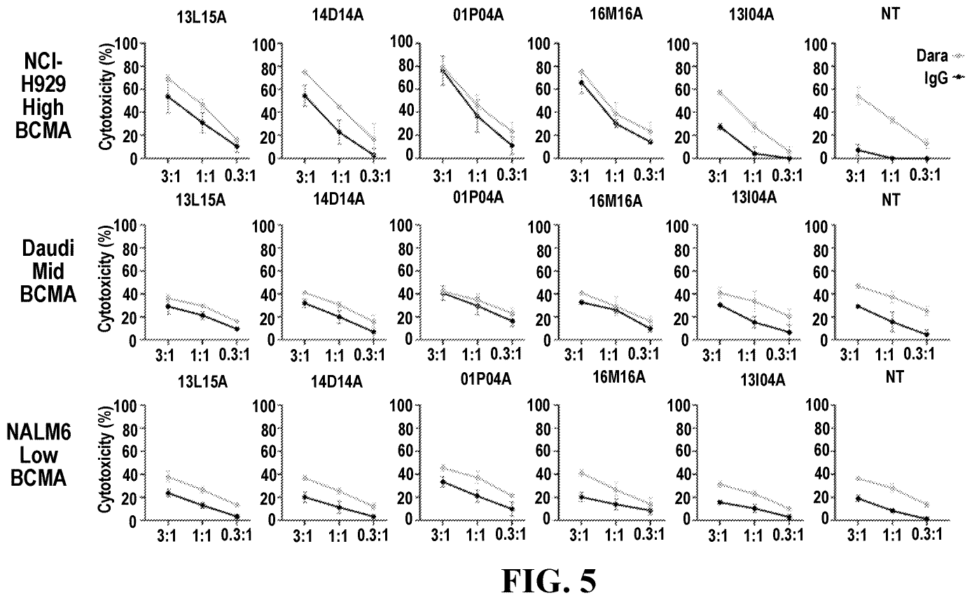

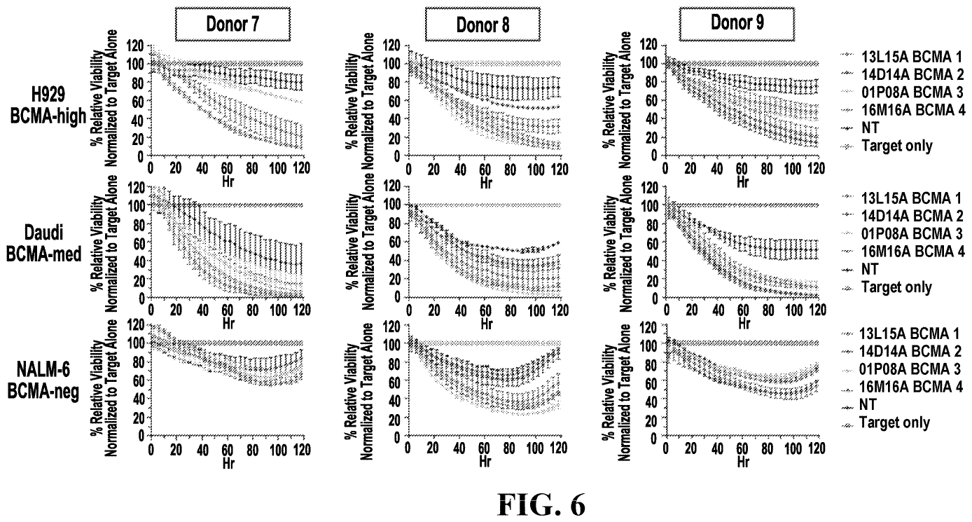

4-hour ADCC assay was performed using calcein AM-stained cells. Effector cells were co-cultured with target cells at three E:T ratios in the presence of 2 μg/ml of daraumumab of IgG1 isotype control. As show in FIG. 5, the Anti-BCMA CAR NK cells demonstrated concentration-dependent short term cytotoxic activity against the tumor cell lines.

Long term cytotoxicity of the CAR NK cells was assessed by fluorometric assay (an Incucyte Live-Cell analysis system which images the NK and target cell co-culture over time) at a low E:T ratio (of 0.3:1). Target cells were stained with 30 μM calcein-AM (Molecular probe) for 1 hour at 37° C. A sample of the CAR NK cells and the labeled tumor cells were co-cultured in a 96-well plate in triplicate at 37° C. and 5% CO2 for 4 hours with light protection. RPMI1640 medium containing 10% FBS or 2% triton-X100 was added to the targets to provide spontaneous and maximum release. RPMI1640 medium containing 10% FBS or 2% triton-X100 was added to each well to determine background fluorescence. The measurement of fluorescence was conducted at excitation of 485 nm and emission 535 nm with a florescent reader. The percent specific cytotoxicity was calculated by the following formula.

$$\% Specific cytotoxicity = 100 \times \frac{\% specific death - \% spontaneous death}{100 - \% spontaneous death}$$

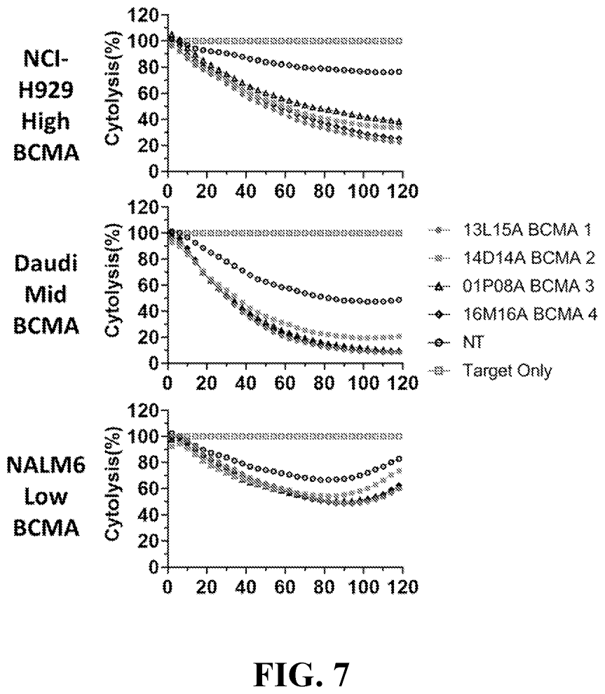

Anti-BCMA CAR NK cells demonstrated potent long-term cytotoxic activity against NCI-H929. Daudi, and NALM-6 cancer cell lines over the 3-day timeframe (FIG. 6, FIG. 7). Cytotoxic activity of anti-BCMA CAR NK was greater than the donor-matched, non-engineered, eHuT-78-expanded cord-blood derived NK cells (CBNK) in co-cultures with NCI-H929 or Daudi. These results indicate that anti-BCMA CAR NK cells have potent cytotoxic activity against BCMA+ cancer cell lines, even at low E:T ratios.

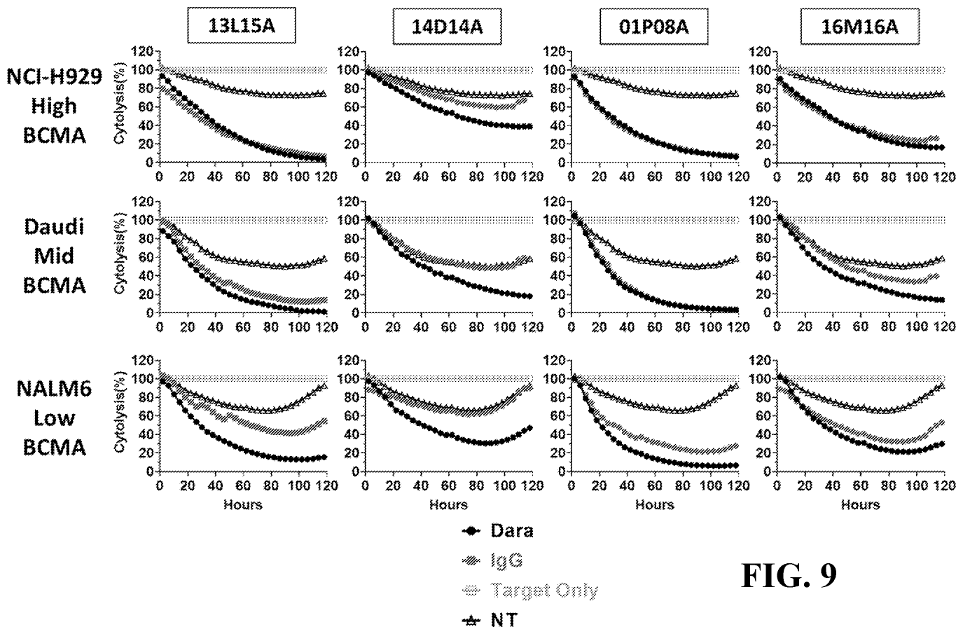

Further long-term cytotoxicity assays were carried out with the anti-BCMA CAR NK cells in the presence of daratumumab (at E:T 0.3:1). Little to no enhancement of cytotoxicity was observed against H929 and Daudi cells when daratumumab was added, due to saturation of BCMA-CAR activity. However, daratumumab-mediated ADCC was observed against NALM-6 cells, which are BCMA-negative. This demonstrates that the combination of anti-BCMA CAR therapy with a CD38 targeting antibody (e.g., daratumumab) can be used for cytotoxicity against both BCMA+ and BCMA− cells (e.g., in a heterogeneous tumor environment where some cells are BCMA+ and some are BCMA−) (FIG. 9).

### 3. Soluble BCMA Interference Assays

Assays were carried out to determine if the presence of soluble BCMA protein (50 nM) interferes with anti-BCMA CAR activity and whether daratumumab can maintained enhanced anti-tumor activity.

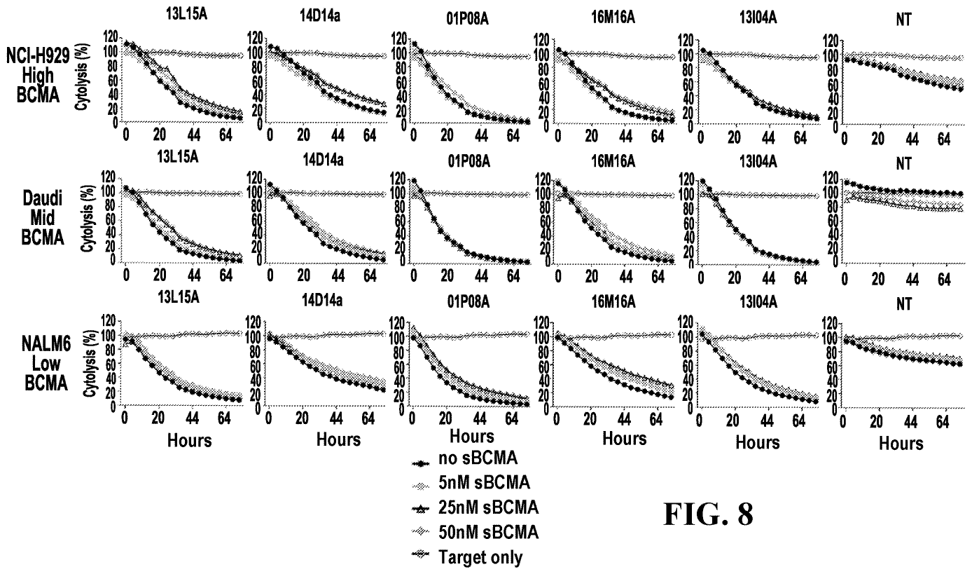

As shown in FIGS. 8, anti-BCMA CAR NK cells demonstrated different levels of tolerance toward soluble BCMA (sBCMA) when co-cultured against NCI-H929, Daudi, and NALM-6 cancer cell lines over the 3-day timeframe, while non-engineered CBNK cells was not impacted. Overall, tumor killing of all BCMA CAR clones was slightly inhibited in the presence of 50 nM soluble BCMA, but BCMA CAR specific activity was not completely abrogated. The addition of daratumumab further maintained enhanced tumor-killing even in the presence of 50 nM soluble BCMA.

### 4. Degranulation and Cyotkine Secretion

BCMA CAR NK candidates were co-cultured with target tumor cells (NCI-H929, Daudi and NALM-6). Expression of degranulation markers by BCMA CAR NK in response to stimulation with tumor cells was measured by flow cytometry. Cytokine secretion in response to co-culture of BCMA CAR NK with target tumor cells was assessed by using the electrochemiluminescence platform by MSD (Meso Scale Discovery).

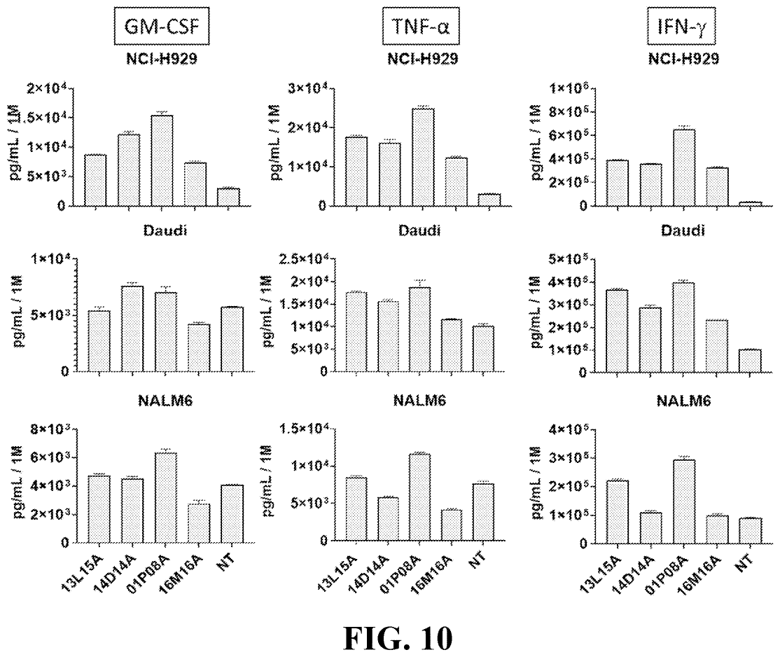

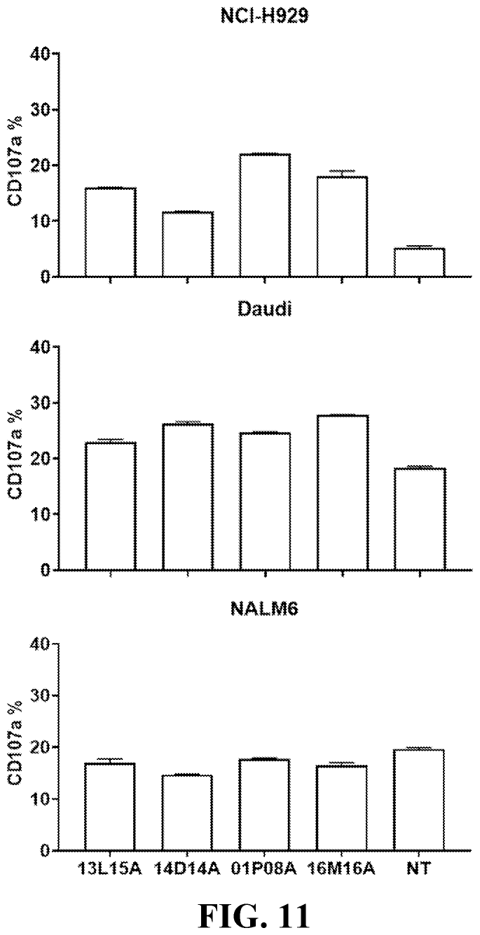

In line with cytotoxic activity, co-culturing of BCMA CAR NK with various cancer cell lines (NCI-H929. Daudi and NALM-6) at a 1:1 Effector: Target (E:T) ratio resulted in increased expression of a marker of degranulation (CD107a) (FIG. 10) and production of effector cytokines (IFN-γ, TNFα, GM-CSF) (FIG. 11) over the non-transduced control CBNK (NT). Further, there was an enhancement of IFN-γ and TNFα production and CD107a expression in NK cells co-cultured with BCMA+ cancer cell lines (i.e. NCI-H929) compared to the non-transduced CBNK. This enhancement was not observed with the non-BCMA-expressing NALM-6 cancer cell line. These results confirm BCMA CAR NK activity in response to co-culture with BCMA+ tumor cells.

### 5. Serial Rechallenge

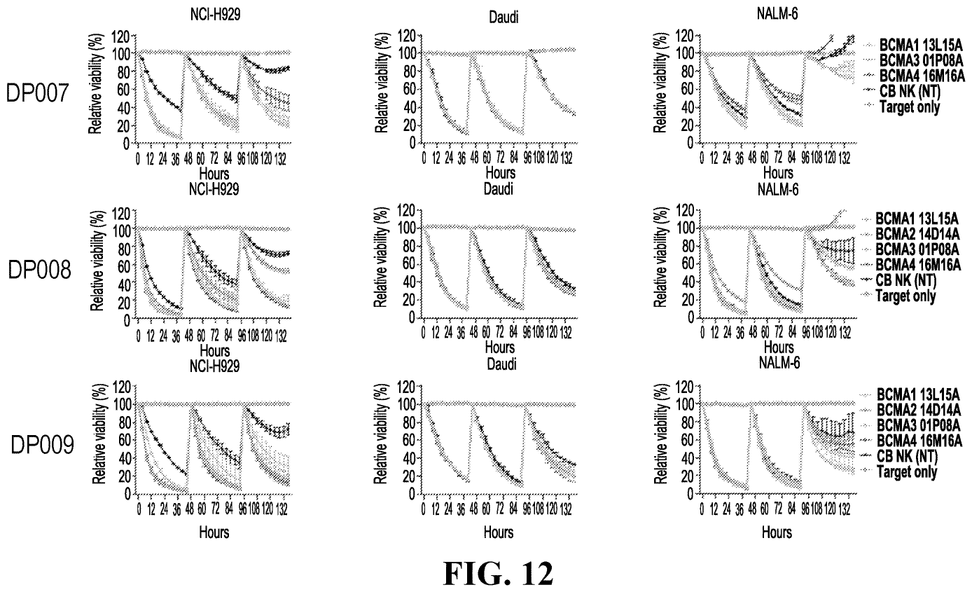

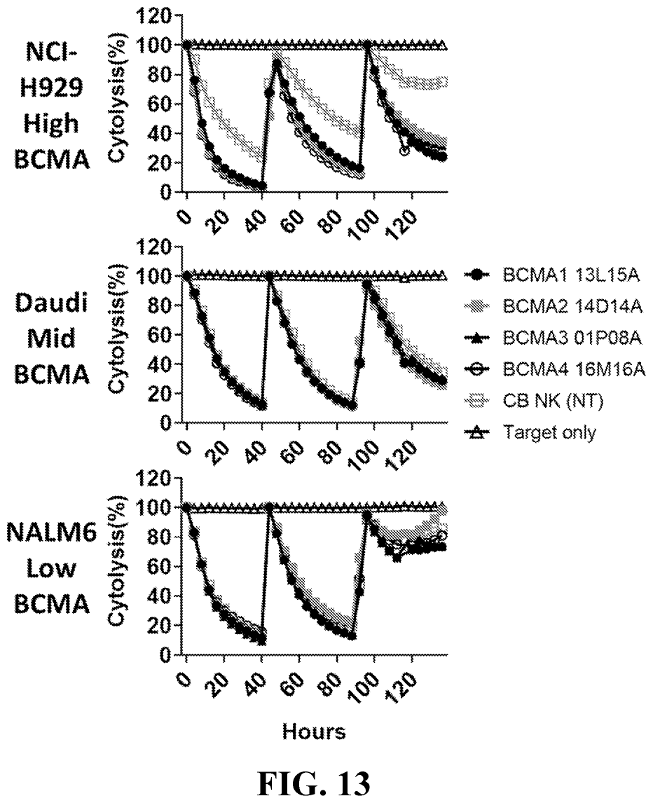

A serial rechallenge assay was carried out using Incucyte to evaluate the anti-tumor capacity of recovered anti-BCMA CAR-NK cells (13L15A, 14D14A, 01P08A, and 16M16A) by serially rechallenging with target cells every 48 hours. Thawed CAR-NK cells were incubated in RPMI-1640 with 10% FBS and IL-2 (2501U/mL) for 24 hours (day 0). Dead cells were removed and assays were set up with 10,000 effector and 10,000 target cells per well supplemented with IL-2 125 IU/mL (day 1). Tumor challenge was repeated on days 3 and 5, and the assay ended on day 7. Clones 13L15A, 0108A, 16M16A had higher cytotoxicity after 3 cycles of tumor re-challenge when co-cultured with NCI-H929 for at least 2 donors (FIG. 12, FIG. 13). These results demonstrating effective killing after three rounds of tumor rechallenge with BCMA expressing targets.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An anti-BCMA antibody or antigen binding fragment thereof comprising:
a CDR-L1 having the amino acid sequence of SEQ ID NO: 7, a CDR-L2 having the amino acid sequence of SEQ ID NO: 8, a CDR-L3 having the amino acid sequence of SEQ ID NO: 9, a CDR-H1 having the amino acid sequence of SEQ ID NO: 10, a CDR-H2 having the amino acid sequence of SEQ ID NO: 11, and a CDR-H3 having the amino acid sequence of SEQ ID NO: 12.

2. The anti-BCMA antibody or antigen binding fragment thereof of claim 1, wherein the CDR-L1, CDR-L2, CDR-L3, CDR-H1, CDR-H2, and CDR-H3 are determined by IMGT numbering.

3. An anti-BCMA antibody or antigen binding fragment thereof comprising:
a CDR-L1 having the amino acid sequence of SEQ ID NO: 13, a CDR-L2 having the amino acid sequence of SEQ ID NO: 14, a CDR-L3 having the amino acid sequence of SEQ ID NO: 9, a CDR-H1 having the amino acid sequence of SEQ ID NO: 15, a CDR-H2 having the amino acid sequence of SEQ ID NO: 16, and a CDR-H3 having the amino acid sequence of SEQ ID NO: 17.

4. The anti-BCMA antibody or antigen binding fragment thereof of claim 3, wherein the CDR-L1, CDR-L2, CDR-L3, CDR-H1, CDR-H2, and CDR-H3 are determined by Kabat numbering.

5. The anti-BCMA antibody or antigen binding fragment of any one of claims 1-4, wherein the antibody or antigen binding fragment thereof comprises a light chain variable (V_{L}) region comprising the amino acid sequence of SEQ ID NO: 18 and a heavy chain variable (V_{H}) region comprising the amino acid sequence of SEQ ID NO: 19.

6. The anti-BCMA antibody or antigen binding fragment of any one of claims 1-5, wherein the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 18 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 19.

7. An anti-BCMA antibody or antigen binding fragment thereof comprising:
a CDR-L1 having the amino acid sequence of SEQ ID NO: 20, a CDR-L2 having the amino acid sequence WAS, a CDR-L3 having the amino acid sequence of SEQ ID NO: 21, a CDR-H1 having the amino acid sequence of SEQ ID NO: 22, a CDR-H2 having the amino acid sequence of SEQ ID NO: 23, and a CDR-H3 having the amino acid sequence of SEQ ID NO: 24.

8. The anti-BCMA antibody or antigen binding fragment thereof of claim 7, wherein the CDR-L1, CDR-L2, CDR-L3, CDR-H1, CDR-H2, and CDR-H3 are determined by IMGT numbering.

9. An anti-BCMA antibody or antigen binding fragment thereof comprising:
a CDR-L1 having the amino acid sequence of SEQ ID NO: 25, a CDR-L2 having the amino acid sequence of SEQ ID NO: 26, a CDR-L3 having the amino acid sequence of SEQ ID NO: 21, a CDR-H1 having the amino acid sequence of SEQ ID NO: 27, a CDR-H2 having the amino acid sequence of SEQ ID NO: 28, and a CDR-H3 having the amino acid sequence of SEQ ID NO: 29.

10. The anti-BCMA antibody or antigen binding fragment thereof of claim 9, wherein the CDR-L1, CDR-L2, CDR-L3, CDR-H1, CDR-H2, and CDR-H3 are determined by Kabat numbering.

11. The anti-BCMA antibody or antigen binding fragment of any one of claims 7-10, wherein the antibody or antigen binding fragment thereof comprises a light chain variable (V_{L}) region comprising the amino acid sequence of SEQ ID NO: 30 and a heavy chain variable (V_{H}) region comprising the amino acid sequence of SEQ ID NO: 31.

12. The anti-BCMA antibody or antigen binding fragment of any one of claims 7-11, wherein the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 30 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 31.

13. An anti-BCMA antibody or antigen binding fragment thereof comprising:
a CDR-L1 having the amino acid sequence of SEQ ID NO: 32, a CDR-L2 having the amino acid sequence SEQ ID NO: 8, a CDR-L3 having the amino acid sequence of SEQ ID NO: 33, a CDR-H1 having the amino acid sequence of SEQ ID NO: 34, a CDR-H2 having the amino acid sequence of SEQ ID NO: 35, and a CDR-H3 having the amino acid sequence of SEQ ID NO: 36.

14. The anti-BCMA antibody or antigen binding fragment thereof of claim 13, wherein the CDR-L1, CDR-L2, CDR-L3, CDR-H1, CDR-H2, and CDR-H3 are determined by IMGT numbering.

15. An anti-BCMA antibody or antigen binding fragment thereof comprising:
a CDR-L1 having the amino acid sequence of SEQ ID NO: 37, a CDR-L2 having the amino acid sequence of SEQ ID NO: 14, a CDR-L3 having the amino acid sequence of SEQ ID NO: 33, a CDR-H1 having the amino acid sequence of SEQ ID NO: 38, a CDR-H2 having the amino acid sequence of SEQ ID NO: 39, and a CDR-H3 having the amino acid sequence of SEQ ID NO: 40.

16. The anti-BCMA antibody or antigen binding fragment thereof of claim 15, wherein the CDR-L1, CDR-L2, CDR-L3, CDR-H1, CDR-H2, and CDR-H3 are determined by Kabat numbering.

17. The anti-BCMA antibody or antigen binding fragment of any one of claims 13-16, wherein the antibody or antigen binding fragment thereof comprises a light chain variable (V_{L}) region comprising the amino acid sequence of SEQ ID NO: 41 and a heavy chain variable (V_{H}) region comprising the amino acid sequence of SEQ ID NO: 42.

18. The anti-BCMA antibody or antigen binding fragment of any one of claims 13-17, wherein the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 41 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 42.

19. An anti-BCMA antibody or antigen binding fragment thereof comprising:
a CDR-L1 having the amino acid sequence of SEQ ID NO: 43, a CDR-L2 having the amino acid sequence LGS, a CDR-L3 having the amino acid sequence of SEQ ID NO: 44, a CDR-H1 having the amino acid sequence of SEQ ID NO: 10, a CDR-H2 having the amino acid sequence of SEQ ID NO: 45, and a CDR-H3 having the amino acid sequence of SEQ ID NO: 46.

20. The anti-BCMA antibody or antigen binding fragment thereof of claim 19, wherein the CDR-L1, CDR-L2, CDR-L3, CDR-H1, CDR-H2, and CDR-H3 are determined by IMGT numbering.

21. An anti-BCMA antibody or antigen binding fragment thereof comprising:
a CDR-L1 having the amino acid sequence of SEQ ID NO: 47, a CDR-L2 having the amino acid sequence of SEQ ID NO: 48, a CDR-L3 having the amino acid sequence of SEQ ID NO: 44, a CDR-H1 having the amino acid sequence of SEQ ID NO: 49, a CDR-H2 having the amino acid sequence of SEQ ID NO: 50, and a CDR-H3 having the amino acid sequence of SEQ ID NO: 51.

22. The anti-BCMA antibody or antigen binding fragment thereof of claim 21, wherein the CDR-L1, CDR-L2, CDR-L3, CDR-H1, CDR-H2, and CDR-H3 are determined by Kabat numbering.

23. The anti-BCMA antibody or antigen binding fragment of any one of claims 19-22, wherein the antibody or antigen binding fragment thereof comprises a light chain variable (V_{L}) region comprising the amino acid sequence of SEQ ID NO: 52 and a heavy chain variable (V_{H}) region comprising the amino acid sequence of SEQ ID NO: 53.

24. The anti-BCMA antibody or antigen binding fragment of any one of claims 19-23, wherein the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 52 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 53.

25. An anti-BCMA antibody or antigen binding fragment thereof comprising:
a CDR-L1 having the amino acid sequence of SEQ ID NO: 43, a CDR-L2 having the amino acid sequence LGS, a CDR-L3 having the amino acid sequence of SEQ ID NO: 44, a CDR-H1 having the amino acid sequence of SEQ ID NO: 10, a CDR-H2 having the amino acid sequence of SEQ ID NO: 45, and a CDR-H3 having the amino acid sequence of SEQ ID NO: 46.

26. The anti-BCMA antibody or antigen binding fragment thereof of claim 25, wherein the CDR-L1, CDR-L2, CDR-L3, CDR-H1, CDR-H2, and CDR-H3 are determined by IMGT numbering.

27. An anti-BCMA antibody or antigen binding fragment thereof comprising:
a CDR-L1 having the amino acid sequence of SEQ ID NO: 47, a CDR-L2 having the amino acid sequence of SEQ ID NO: 48, a CDR-L3 having the amino acid sequence of SEQ ID NO: 44, a CDR-H1 having the amino acid sequence of SEQ ID NO: 54, a CDR-H2 having the amino acid sequence of SEQ ID NO: 50, and a CDR-H3 having the amino acid sequence of SEQ ID NO: 51.

28. The anti-BCMA antibody or antigen binding fragment thereof of claim 27, wherein the CDR-L1, CDR-L2, CDR-L3, CDR-H1, CDR-H2, and CDR-H3 are determined by Kabat numbering.

29. The anti-BCMA antibody or antigen binding fragment of any one of claims 25-28, wherein the antibody or antigen binding fragment thereof comprises a light chain variable (V_{L}) region comprising the amino acid sequence of SEQ ID NO: 52 and a heavy chain variable (V_{H}) region comprising the amino acid sequence of SEQ ID NO: 55.

30. The anti-BCMA antibody or antigen binding fragment of any one of claims 25-29, wherein the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 52 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 55.

31. An anti-BCMA antibody or antigen binding fragment thereof comprising:
a CDR-L1 having the amino acid sequence of SEQ ID NO: 7, a CDR-L2 having the amino acid sequence SEQ ID NO: 8, a CDR-L3 having the amino acid sequence of SEQ ID NO: 56, a CDR-H1 having the amino acid sequence of SEQ ID NO: 57, a CDR-H2 having the amino acid sequence of SEQ ID NO: 58, and a CDR-H3 having the amino acid sequence of SEQ ID NO: 59.

32. The anti-BCMA antibody or antigen binding fragment thereof of claim 31, wherein the CDR-L1, CDR-L2, CDR-L3, CDR-H1, CDR-H2, and CDR-H3 are determined by IMGT numbering.

33. An anti-BCMA antibody or antigen binding fragment thereof comprising:
a CDR-L1 having the amino acid sequence of SEQ ID NO: 13, a CDR-L2 having the amino acid sequence of SEQ ID NO: 14, a CDR-L3 having the amino acid sequence of SEQ ID NO: 56, a CDR-H1 having the amino acid sequence of SEQ ID NO: 60, a CDR-H2 having the amino acid sequence of SEQ ID NO: 61, and a CDR-H3 having the amino acid sequence of SEQ ID NO: 62.

34. The anti-BCMA antibody or antigen binding fragment thereof of claim 33, wherein the CDR-L1, CDR-L2, CDR-L3, CDR-H1, CDR-H2, and CDR-H3 are determined by Kabat numbering.

35. The anti-BCMA antibody or antigen binding fragment of any one of claims 31-34, wherein the antibody or antigen binding fragment thereof comprises a light chain variable (V_{L}) region comprising the amino acid sequence of SEQ ID NO: 63 and a heavy chain variable (V_{H}) region comprising the amino acid sequence of SEQ ID NO: 64.

36. The anti-BCMA antibody or antigen binding fragment of any one of claims 31-35, wherein the antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 63 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 64.

37. The anti-BCMA antibody or antigen binding fragment thereof of any one of claims 1-36, wherein the antibody or antigen binding fragment thereof is a human antibody, a humanized antibody, or a chimeric antibody.

38. The anti-BCMA antibody or antigen binding fragment thereof of any one of claims 1-37, wherein the antibody or antigen binding fragment thereof is an antigen binding fragment selected from the group consisting of a Fab, a Fab′, a F(ab′)₂, a Fv, and a scFv.

39. The anti-BCMA antigen binding fragment of claim 38, wherein the antigen binding fragment is a scFv.

40. The anti-BCMA antigen binding fragment of claim 39, wherein the V_{L} region is amino-terminal to the V_{H} region.

41. The anti-BCMA antigen binding fragment of claim 39, wherein the V_{L} region is carboxy-terminal to the V_{H} region.

42. The anti-BCMA antigen binding fragment of any one of claims 39-41, wherein the V_{L} region is joined to the V_{H} region via a flexible linker.

43. The anti-BCMA antigen binding fragment of claim 42, wherein the flexible linker comprises the amino acid sequence set forth in SEQ ID NO: 66.

44. The anti-BCMA antigen binding fragment of any one of claims 39-43, wherein the scFv comprises the amino acid sequence set forth in SEQ ID NO: 68.

45. The anti-BCMA antigen binding fragment of any one of claims 39-43, wherein the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 68.

46. The anti-BCMA antigen binding fragment of any one of claims 39-43, wherein the scFv comprises the amino acid sequence set forth in SEQ ID NO: 69.

47. The anti-BCMA antigen binding fragment of any one of claims 39-43, wherein the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 69.

48. The anti-BCMA antigen binding fragment of any one of claims 39-43, wherein the scFv comprises the amino acid sequence set forth in SEQ ID NO: 70.

49. The anti-BCMA antigen binding fragment of any one of claims 39-43, wherein the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 70.

50. The anti-BCMA antigen binding fragment of any one of claims 39-43, wherein the scFv comprises the amino acid sequence set forth in SEQ ID NO: 71.

51. The anti-BCMA antigen binding fragment of any one of claims 39-43, wherein the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 71.

52. The anti-BCMA antigen binding fragment of any one of claims 39-43, wherein the scFv comprises the amino acid sequence set forth in SEQ ID NO: 72.

53. The anti-BCMA antigen binding fragment of any one of claims 39-43, wherein the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 72.

54. An anti-BCMA chimeric antigen receptor (CAR) comprising:
an extracellular antigen binding domain comprising an anti-BCMA antibody or antigen binding fragment thereof; and
an intracellular signaling region comprising an OX40L intracellular signaling domain.

55. The anti-BCMA CAR of claim 54, wherein the OX40L intracellular signaling domain comprises an amino acid sequence set forth in SEQ ID NO: 79, SEQ ID NO: 80, or SEQ ID NO: 81.

56. The anti-BCMA CAR of claim 54 or claim 55, wherein the OX40L intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 79, SEQ ID NO: 80, or SEQ ID NO: 81.

57. The anti-BCMA CAR of any one of claims 54-56, wherein the extracellular antigen binding domain comprising an anti-BCMA antibody or antigen binding fragment thereof comprises the anti-BCMA antibody or antigen binding fragment thereof of any one of claims 1-56.

58. An anti-BCMA chimeric antigen receptor (CAR) comprising an extracellular antigen binding domain comprising the anti-BCMA antibody or antigen binding fragment thereof of any one of claims 1-56 and an intracellular region.

59. The anti-BCMA CAR of any one of claims 54-58, wherein the anti-BCMA CAR specifically binds to a B-cell maturation antigen (BCMA) protein.

60. The anti-BCMA CAR of claim 58, wherein the BCMA protein comprises the amino acid sequence of SEQ ID NO: 1 or SEQ ID NO: 2.

61. The anti-BCMA CAR of any one of claims 54-60, wherein the CAR comprises a transmembrane region.

62. The anti-BCMA CAR of claim 61, wherein the transmembrane region comprises a CD28 transmembrane domain.

63. The anti-BCMA CAR of claim 62, wherein the CD28 transmembrane domain comprises the amino acid sequence set forth in SEQ ID NO: 77.

64. The anti-BCMA CAR of claim 62 or claim 63, further comprising a hinge domain between the extracellular antigen binding domain and the transmembrane domain.

65. The anti-BCMA CAR of claim 64, wherein the hinge domain comprises at least a portion of a CD8α hinge domain.

66. The anti-BCMA CAR of claim 65, wherein the CD8α hinge domain comprises an amino acid sequence set forth in SEQ ID NO: 76.

67. The anti-BCMA CAR of claim 66, wherein the CD8α hinge domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 76.

68. The anti-BCMA CAR of any one of claims 54-67, wherein the intracellular signaling region further comprises a CD28 intracellular signaling domain.

69. The anti-BCMA CAR of any one of claims 54-68, wherein the intracellular signaling region further comprises a CD3-zeta (CD3ζ) signaling domain.

70. The anti-BCMA CAR of any one of claims 54-67, wherein the intracellular signaling region further comprises a CD28 intracellular signaling domain and a CD3-zeta signaling domain.

71. The anti-BCMA CAR of claim 68 or claim 70, wherein the CD28 intracellular signaling domain comprises an amino acid sequence set forth in SEQ ID NO: 78.

72. The anti-BCMA CAR of claim 68 or claim 70, wherein the CD28 intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 78.

73. The anti-BCMA CAR of claim 69 or claim 70, wherein the CD3-zeta intracellular signaling domain comprises an amino acid sequence set forth in SEQ ID NO: 82.

74. The anti-BCMA CAR of claim 69 or claim 70, wherein the CD3-zeta intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 82.

75. The anti-BCMA CAR of any one of claims 54-74, wherein the intracellular signaling region comprises an amino acid sequence set forth in SEQ ID NO: 83.

76. The anti-BCMA CAR of any one of claims 54-74, wherein the intracellular signaling region comprises an amino acid sequence having or having at least 85%, 86%, 87%, 88%, 89%, 90%, 91%, 92%, 93%, 94%, 95%, 96%, 97%, 98%, or 99% identity to SEQ ID NO: 83.

77. The anti-BCMA CAR of any one of claims 54-76, comprising the amino acid sequence set forth in any one of SEQ ID NO: 88, SEQ ID NO: 90, SEQ ID NO: 92, SEQ ID NO: 94, or SEQ ID NO: 96.

78. The anti-BCMA CAR of any one of claims 54-76, comprising an amino acid sequence having or having at least 85%, 86%, 87%, 88%, 89%, 90%, 91%, 92%, 93%, 94%, 95%, 96%, 97%, 98%, or 99% identity an amino acid sequence set forth in any one of SEQ ID NO: 88, SEQ ID NO: 90, SEQ ID NO: 92, SEQ ID NO: 94, or SEQ ID NO: 96.

79. A polynucleotide comprising a nucleic acid encoding the anti-BCMA CAR of any one of claims 54-78.

80. The polynucleotide of claim 79, wherein the nucleic acid encoding the anti-BCMA CAR comprises the nucleic acid sequence set forth in any one of SEQ ID NO: 89, SEQ ID NO: 91, SEQ ID NO: 93, SEQ ID NO: 95, or SEQ ID NO: 97.

81. The polynucleotide of claim 79 or claim 80, further comprising a nucleic acid encoding an IL-15.

82. The polynucleotide of claim 81, wherein the IL-15 comprises the amino acid sequence set forth in SEQ ID NO: 86.

83. The polynucleotide of claim 82, wherein the IL-15 is encoded by a nucleic acid comprising SEQ ID NO: 87.

84. The polynucleotide of any one of claims 81-83, wherein the polynucleotide encodes a polyprotein comprising the CAR and the IL-15.

85. The polynucleotide of any one of claims 81-84, further comprising a nucleic acid encoding a self-cleaving peptide, optionally a T2A self-cleaving peptide.

86. The polynucleotide of claim 85, wherein the CAR is joined to the IL-15 by the self-cleaving peptide.

87. The polynucleotide of claim 86, wherein the self-cleaving peptide is capable of inducing ribosomal skipping between the CAR and the IL-15.

88. The polynucleotide of claim 86 or claim 87, wherein the self-cleaving peptide comprises the sequence set forth in SEQ ID NO: 84.

89. The polynucleotide of any one of claims 86-88, where the self-cleaving peptide is encoded by the sequence set forth in SEQ ID NO: 85.

90. The polynucleotide of any one of claims 79-89, further comprising a nucleic acid encoding a signal sequence.

91. The polynucleotide of claim 90, wherein the signal sequence comprises the amino acid sequence set forth in SEQ ID NO: 74.

92. The polynucleotide of claim 91, wherein the nucleic acid encoding the signal sequence comprises the nucleic acid sequence set forth in SEQ ID NO: 75.

93. The polynucleotide of any one of claims 79-92, wherein the polynucleotide encodes a polyprotein comprising the amino acid sequence set forth in SEQ ID NO: 98, SEQ ID NO: 99, SEQ ID NO: 100, SEQ ID NO: 101, or SEQ ID NO: 102.

94. The polynucleotide of any one of claims 79-93, wherein the polynucleotide comprises the nucleic acid sequence set forth in SEQ ID NO: 103, SEQ ID NO: 105, SEQ ID NO: 107, SEQ ID NO: 109, or SEQ ID NO: 111.

95. A vector comprising the polynucleotide of any one of claims 79-94.

96. The vector of claim 95, wherein the vector is a viral vector.

97. The vector of claim 97, wherein the viral vector is a retroviral vector or a lentiviral vector.

98. A cell comprising the polynucleotide of any one of claims 79-94 or the vector of any one of claims 95-97.

99. A cell expressing the chimeric antigen receptor encoded by the polynucleotide of any one of claims 79-94 or the vector of any one of claims 95-97.

100. A cell expressing the chimeric antigen receptor and the IL-15 encoded by the polynucleotide of any one of claims 81-94 or the vector of any one of claims 95-97.

101. The cell of any one of claims 98-100, wherein the cell is a lymphocyte.

102. The cell of claim 101, wherein the lymphocyte is a natural killer (NK) cell.

103. The cell of claim 101, wherein the lymphocyte is a T cell.

104. The cell of any one of claims 98-103, wherein the cell is a human cell.

105. The cell of any one of claims 98-104, wherein the cell is a primary cell obtained from a subject.

106. The cell of any one of claims 98-105, wherein the cell is a primary cell obtained from cord blood.

107. The cell of any one of claims 98-106, wherein the cell comprises a KIR-B haplotype.

108. The cell of any one of claims 98-107, wherein the cell express CD16 having the V/V polymorphism at F158.

109. A population of cells comprising a plurality of the cells of any one of claims 98-108.

110. The population of cells of claim 109, wherein the population comprises expanded natural killer cells.

111. The population of cells of claim 110, wherein the expanded natural killer cells are expanded umbilical cord blood natural killer cells.

112. The population of cells of claim 110 or claim 111, wherein at least 70% of the NK cells are CD56+ and CD16+.

113. The population of cells of any one of claims 110-112, wherein at least 85% of the NK cells are CD56+ and CD3−.

114. The population of cells of any one of claims 110-113, wherein 1% or less of the NK cells are CD3+, 1% or less of the NK cells are CD19+ and 1% or less of the NK cells are CD14+.

115. The population of cells of any one of claims 110-114, wherein the population of expanded natural killer cells comprises at least 60%, e.g., at least 70%, at least 80%, at least 90% at least 95%, at least 99%, or 100% CD16+ cells.

116. The population of cells of any one of claims 110-115, wherein the population of expanded natural killer cells comprises at least 60%, e.g., at least 70%, at least 80%, at least 90% at least 95%, at least 99%, or 100% NKG2D+ cells.

117. The population of cells of any one of claims 110-116, wherein the population of expanded natural killer cells comprises at least 60%, e.g., at least 70%, at least 80%, at least 90% at least 95%, at least 99%, or 100% NKp46+ cells.

118. The population of cells of any one of claims 110-117, wherein the population of expanded natural killer cells comprises at least 60%, e.g., at least 70%, at least 80%, at least 90% at least 95%, at least 99%, or 100% NKp30+ cells.

119. The population of cells of any one of claims 110-118, wherein the population of expanded natural killer cells comprises at least 60%, e.g., at least 70%, at least 80%, at least 90% at least 95%, at least 99%, or 100% DNAM-1+ cells.

120. The population of cells of any one of claims 110-119, wherein the population of expanded natural killer cells comprises at least 60%, e.g., at least 70%, at least 80%, at least 90% at least 95%, at least 99%, or 100% NKp44+ cells.

121. The population of cells of any one of claims 110-120, wherein the population of expanded natural killer cells comprises less than 20%, e.g., 10% or less, 5% or less, 1% or less, 0.5% or less, or 0% CD3+ cells.

122. The population of cells of any one of claims 110-121, wherein the population of expanded natural killer cells comprises less than 20% or less, e.g., 10% or less, 5% or less, 1% or less, 0.5% or less, or 0% CD14+ cells.

123. The population of cells of any one of claims 110-122, wherein the population of expanded natural killer cells comprises less than 20% or less, e.g., 10% or less, 5% or less, 1% or less, 0.5% or less, or 0% CD19+ cells.

124. The population of cells of any one of claims 110-123, wherein the population of expanded natural killer cells comprises less than 40% or less, 35% or less, 30% or less, 25% or less, or 20% or less, e.g., 10% or less, 5% or less, 1% or less, 0.5% or less, or 0% CD38+ cells.

125. The population of cells of any one of claims 110-124, wherein the natural killer cells do not comprise a CD16 transgene.

126. The population of cells of any one of claims 110-125, wherein the natural killer cells do not express an exogenous CD16 protein.

127. The population of cells of any one of claims 110-126, wherein the expanded natural killer cells are derived from a single umbilical cord blood donor.

128. The population of cells of any one of claims 110-127, wherein the umbilical cord blood is from a donor with the KIR-B haplotype and homozygous for the CD16 158V polymorphism.

129. The population of cells of any one of claims 110-128, wherein the population of NK cells is produced by a method comprising expanding the natural killer cells from umbilical cord blood at least 10,000 fold, e.g., 15,000 fold, 20,000 fold, 25,000 fold, 30,000 fold, 35,000 fold, 40,000 fold, 45.000 fold, 50,000 fold, 55,000 fold, 60,000 fold, 65,000 fold, or 70.000 fold.

130. The population of cells of any one of claims 110-129, wherein the population of expanded natural killer cells is not enriched or sorted after expansion.

131. The population of cells of any one of claims 111-130, wherein the percentage of NK cells expressing CD16 in the population of expanded natural killer cells is the same or higher than the percentage of natural killer cells in the seed cells from umbilical cord blood.

132. The population of cells of any one of claims 111-131, wherein the percentage of NK cells expressing NKG2D in the population of expanded natural killer cells is the same or higher than the percentage of natural killer cells in the seed cells from umbilical cord blood.

133. The population of cells of any one of claims 111-132, wherein the percentage of NK cells expressing NKp30 in the population of expanded natural killer cells is the same or higher than the percentage of natural killer cells in the seed cells from umbilical cord blood.

134. The population of cells of any one of claims 111-133, wherein the percentage of NK cells expressing NKp44 in the population of expanded natural killer cells is the same or higher than the percentage of natural killer cells in the seed cells from umbilical cord blood.

135. The population of cells of any one of claims 111-134, wherein the percentage of NK cells expressing NKp46 in the population of expanded natural killer cells is the same or higher than the percentage of natural killer cells in the seed cells from umbilical cord blood.

136. The population of cells of any one of claims 111-135, wherein the percentage of NK cells expressing DNAM-1 in the population of expanded natural killer cells is the same or higher than the percentage of natural killer cells in the seed cells from umbilical cord blood.

137. The population of cells of any one of claims 110-136, wherein the population of NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

138. The population of cells of any one of claims 109-137, wherein at least 10%, 20%, 30%, 40%, 50%, 60%, 70%, 80%, 85%, 90%; 95%, 96%, 97%, 98%, or 99% of the cells comprise the CAR of any one of claims 58-78, the polynucleotide of any one of claims 79-94, or the vector of any one of claims 95-97.

139. The population of cells of any one of claims 109-137, wherein at least 10%, 20%, 30%, 40%, 50%, 60%, 70%, 80%, 85%, 90%; 95%, 96%, 97%, 98%, or 99% of the cells express the CAR of any one of claims 58-78, the polynucleotide of any one of claims 79-94, or the vector of any one of claims 95-97.

140. A pharmaceutical composition comprising the cell of any one of claims 98-108 or the population of cells of any one of claims 109-139.

141. The pharmaceutical composition of claim 140, further comprising a pharmaceutically acceptable excipient.

142. A frozen vial comprising the pharmaceutical composition of claim 141.

143. A method of treatment comprising administering the antibody or antigen-binding fragment thereof of any one of claims 1-53, the anti-BCMA chimeric antigen receptor of any one of claims 54-78, the cell of any one of claims 98-108, the population of cells of any one of claims 109-139, or the composition of any one of claims 140-141 to a subject having a disease or condition associated with BCMA.

144. Use of the antibody or antigen-binding fragment thereof of any one of claims 1-53, the anti-BCMA chimeric antigen receptor of any one of claims 54-78, the cell of any one of claims 98-108, the population of cells of any one of claims 109-139, or the composition of any one of claims 140-141 in the manufacture of a medicament for treating a disease or condition associated with BCMA.

145. Use of the antibody or antigen-binding fragment thereof of any one of claims 1-53, the anti-BCMA chimeric antigen receptor of any one of claims 54-78, the cell of any one of claims 98-108, the population of cells of any one of claims 109-139, or the composition of any one of claims 140-141 for treating a disease or condition associated with BCMA.

146. The method or use of any one of claims 143-145, wherein the disease or condition associated with BCMA is cancer.

147. The method or use of claim 146, wherein the cancer is a BCMA+ cancer.

148. The method or use of claim 147, wherein the BCMA+ cancer is or comprises a hematopoietic neoplastic disorder expressing BCMA.

149. The method or use of any one of claims 146-148, wherein the cancer is or comprises neoplastic cells of hematopoietic origin.

150. The method or use of any one of claims 146-149, wherein the cancer arises from a lymphoid lineage, or a precursor cell thereof.

151. The method or use of any one of claims 146-150, wherein the cancer arises from a plasma cell, or a precursor cell thereof.

152. The method or use of any one of claims 143-151, wherein the disease or condition associated with BCMA is a myeloma, smoldering myeloma, plasmacytoma, multiple myeloma, leukemia, or lymphoma.

153. The method or use of claim 152, wherein the disease or condition associated with BCMA is multiple myeloma.

154. The method or use of claim 153, wherein the multiple myeloma is a high-risk myeloma or a lenalidomide-refractory multiple myeloma.

155. The method or use of any one of claims 143-154, wherein the subject has relapsed after treatment with an anti-CD38 antibody.

156. The method or use of any one of claims 143-155, wherein the subject has experienced disease progression after treatment with autologous stem cell transplant or chimeric antigen receptor T-cell therapy (CAR-T).

157. The method or use of any one of claims 143-153, wherein the cells are allogenic to the subject.

158. The method or use of any one of claims 143-153, further comprising administering an antibody targeted to CD38.

159. The method or use of claim 158, wherein the antibody is daratumumab, isatuximab, or a biosimilar thereof.

160. The method or use of claim 158, wherein the antibody is daratumumab.

161. The method or use of claim 158, wherein the antibody is isatuximab.

162. The method or use of any one of claims 143-145, wherein the disease or condition associated with BCMA is an autoimmune disorder.

163. The method of claim 162, wherein the autoimmune disorder is selected from Acromegaly, Acquired aplastic anemia, Acquired hemophilia, Primary Agammaglobulinemia, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), Autoimmune hvperlipidemia, Autoimmune hypophysitis, Autoimmune inner ear disease (AIED), Autoimmune lymphoproliferative syndrome (ALPS), Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED), Autoimmune progesterone dermatitis, Autoimmune retinopathy (AIR), Autoimmune sudden sensorineural hearing loss (SNHL), Balo disease, Behget's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA), Cogan's syndrome, Cold agglutinin disease, CREST syndrome|limited cutaneous systemic sclerosis, Crohn's disease (CD), Cronkhite-Canada syndrome (CSS), Cryptogenic organizing pneumonia (COP), Dermatitis herpetiformis, Dermatomyositis, Type 1 Diabetes, Discoid lupus, Dressler's syndrome/postmyocardial infarction/postpericardiotomy syndrome, Eczema/Atopic Dermatitis, Endometriosis, Eosinophilic esophagitis, Eosinophilic fasciitis, Erythema nodosum, Essential mixed cryoglobulinemia, Evans syndrome, Fibrosing alveolitis/Idiopathic pulmonary fibrosis (IPF), Giant cell arteritis/temporal arteritis/Horton's disease, Giant Cell Myocarditis, Glomerulonephritis, Goodpasture's syndrome/anti-GBM/anti-TBM disease, Granulomatosis with polyangiitis (GPA)/Wegener's granulomatosis, Graves disease/thyroid eye disease, Guillain-Barre syndrome (GBS), Hashimoto's thyroiditis/chronic lymphocytic thyroiditis/autoimmune thyroiditis, Henoch-Schönlein purpura/IgA vasculitis, Hidradenitis suppurativa, Hurst's disease/acute hemorrhagic leukoencephalitis (AHLE), Hypogammaglobulinemia, IgA nephropathy/Berger's disease, Immune-mediated necrotizing myopathy (IMNM), Immune thrombocytopenia (ITP)/autoimmune thrombocytopenic purpura/autoimmune thrombocytopenia, Inclusion body myositis, IgG4-related sclerosing disease (ISD), Interstitial cystitis, Juvenile idiopathic arthritis/Adult-onset Still's disease, Juvenile polymyositis|Juvenile dermatomyositis|juvenile myositis, Kawasaki disease, Lambert-Eaton myasthenic syndrome (LEMS), Leukocytoclastic vasculitis, Lichen planus, Lichen sclerosus, Ligneous conjunctivitis, Linear IgA disease (LAD)|linear IgA bullous dermatosis (LABD), Lupus nephritis, Lyme disease/chronic Lyme disease/post-treatment Lyme disease syndrome (PTLDS), Lymphocytic colitis/microscopic colitis, Lymphocytic hypophystitis/autoimmune hypophystitis, Ménière's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Neuromyelitis Optica/Devic's disease, Ocular cicatricial pemphigoid, Opsoclonus-myoclonus syndrome (OMS), Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pernicious anemia, POEMS syndrome, Polyarteritis nodosa, Polymyalgia rheumatica, Polymyositis, Postural orthostatic tachycardia syndrome (POTS), Primary biliary cirrhosis (PBC)/primary biliary cholangitis, Primary sclerosing cholangitis (PSC), Psoriasis, Palmoplantar Pustulosis, Psoriatic arthritis, Pulmonary fibrosis, idiopathic (IPF), Pure red cell aplasia (PRCA), Pyoderma gangrenosum, Rasmussen's encephalitis, Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease, Rheumatic fever, Rheumatoid arthritis, Sarcoidosis, Schmidt syndrome/autoimmune polyendocrine syndrome type II, Scleritis, Scleroderma, Sclerosing Mesenteritis/Mesenteric Panniculitis, Serpiginous choroidopathy, Sjögren's syndrome, Stiff person syndrome (SPS), Small fiber sensory neuropathy, Systemic lupus erythematosus (SLE), Subacute bacterial endocarditis (SBE), Subacute cutaneous lupus, Susac syndrome, Sydenham's chorea, Sympathetic ophthalmia, Takayasu's arteritis (vasculitis), Testicular autoimmunity (vasculitis, orchitis), Tolosa-Hunt syndrome, Transverse myelitis™, Tubulointerstitial nephritis uveitis syndrome (TINU), Ulcerative colitis (UC), Undifferentiated connective tissue disease (UCTD), Uveitis|anterior/intermediate/posterior, Vasculitis, VEXAS Syndrome, Vitiligo, Vogt-Koyanagi-Harada syndrome (VKH), and combinations thereof.
